(19) United States
(12) Patent Application Publication
(10) Pub. No.: US 2026/0366027 A1
(43) Pub. Date: Nov. 19, 2026

(54) COMPOSITIONS AND METHODS COMPRISING SMALL NUCLEAR RNA (SNRNA) TARGETING SOD1

(71) Applicant: Regeneron Pharmaceuticals, Inc., Tarrytown, NY (US)

(72) Inventors: Daniela ROTH, San Diego, CA (US); William Henry BRADFORD, San Diego, CA (US)

(21) Appl. No.: 19/706,523

(22) Filed: Jun. 12, 2026

Related U.S. Application Data
(63) Continuation of application No. PCT/IB2024/062668, filed on Dec. 13, 2024.
(60) Provisional application No. 63/610,756, filed on Dec. 15, 2023.

(51) Int. Cl. C12N 15/113; C07K 14/005; C12N 15/86

(52) U.S. Cl. CPC C12N 15/1137; C07K 14/005; C12N 15/86; C12Y 115/01001; C12N 2310/531; C12N 2750/14122; C12N 2750/14143

(57) ABSTRACT

SnRNA systems targeting SOD1 RNA sequences are disclosed herein. Further disclosed are methods of treating Amyotrophic Lateral Sclerosis.

### RELATED APPLICATIONS

This application is a bypass continuation of International Application No. PCT/IB2024/062668, filed Dec. 13, 2024, which claims the priority to, and benefit of, U.S. Provisional Application No. 63/610,756, filed on Dec. 15, 2023, the contents of each of which are incorporated by reference in their entireties.

### REFERENCE TO AN ELECTRONIC SEQUENCE LISTING

The contents of the electronic sequence listing (REGE_037_C01US_SeqList_ST26.xml; Size: 157,818 bytes; and Date of Creation: Jun. 12, 2026) is herein incorporated by reference in its entirety.

### FIELD OF THE DISCLOSURE

The disclosure is directed to molecular biology, gene therapy, and compositions and methods for modifying expression and activity of RNA molecules.

### BACKGROUND

Small nuclear RNA (snRNA) is one of the smallest types of RNA with an average size of about 150 nucleotides. snRNAs are functional non-coding RNAs. Eukaryotic genomes code for a variety of non-coding RNA such as snRNA, a class of highly abundant RNA, localized in the nucleus with important functions in intron splicing and RNA processing. snRNA, in the pre-mRNA splicing process, are capable of forming ribonucleoprotein particles (snRNPs) along with other proteins. These snRNPs and additional proteins form a large particulate complex (spliceosome) bound to the unspliced pre-mRNA transcripts. In addition to splicing, snRNAs function in nuclear maturation of nascent transcripts, gene expression regulation, as a splice donor in non-canonical systems, and in 3′ end processing of replication-dependent histone mRNAs. U7 snRNA can be programmed to bind and modulate mRNA without exogenous protein expression, which can ultimately decrease the risk of immunogenicity observed with other protein-based gene therapy approaches. Furthermore, the small size of these programmed snRNAs creates an opportunity to develop single vector, highly specific (e.g., allele-specific), single target and multi-targeting gene therapy approaches.

Amyotrophic lateral sclerosis (ALS) also known as Lou Gehrig's disease or motor neuron disease (MND) is a rare and terminal neurodegenerative disease that results in the progressive loss of motor neurons that control voluntary muscles. Early symptoms of ALS include stiff muscles, muscle twitches, gradual increasing weakness, and muscle wasting. Limb-onset ALS begins with weakness in the arms or legs, while bulbar-onset ALS begins with difficulty in speaking or swallowing. Around half of people with ALS develop at least mild difficulties with thinking and behavior, and about 15% develop frontotemporal dementia (FTD). Motor neuron loss continues until the abilities to eat, speak, move, or, lastly, breathe are lost.

ALS can be classified as being either familial or sporadic, depending on whether there is a known family history of the disease and/or whether an ALS-associated genetic mutation has been identified via genetic testing. Mutations in SOD1, encoding superoxide dismutase 1, have been linked to familial amyotrophic lateral sclerosis. In some aspects, SOD1-related ALS can also be referred to as SOD1 ALS. However, several pieces of evidence also show that wild-type SOD1, under conditions of cellular stress, is implicated in a significant fraction of sporadic ALS cases, which represent 90% of ALS patients.

SOD1 is a 32 kDa homodimer which forms a beta barrel (0-barrel) and contains an intramolecular disulfide bond and a binuclear Cu/Zn site in each subunit. This Cu/Zn site holds the copper and a zinc ion and is responsible for catalyzing the disproportionation of superoxide to hydrogen peroxide and dioxygen. SOD1 binds copper and zinc ions and is one of three superoxide dismutases responsible for destroying free superoxide radicals in the body. The encoded isozyme is a soluble cytoplasmic and mitochondrial intermembrane space protein, acting as a homodimer to convert naturally occurring, but harmful, superoxide radicals to molecular oxygen and hydrogen peroxide. Hydrogen peroxide can then be broken down by another enzyme called catalase.

More than 150 SOD1 mutations have been linked to ALS. Common SOD1 mutations include A4V, H46R, and G93A, with others having also been characterized.

ALS is a neurodegenerative disease characterized by selective loss of motor neurons causing muscle atrophy. The DNA oxidation product 8-hydroxyguanosine (8-OHdG) is a well-established marker of oxidative DNA damage. 8-OHdG accumulates in the mitochondria of spinal motor neurons of persons with ALS. In transgenic ALS mice harboring a mutant SOD1 gene, 8-OHdG also accumulates in mitochondrial DNA of spinal motor neurons. These findings suggest that oxidative damage to mitochondrial DNA of motor neurons due to altered SOD1 may be significant factor in the etiology of ALS.

Accordingly, SOD1 RNA targeting snRNA molecules of the disclosure that can alter RNA splicing by promoting exon skipping or inclusion can be used to control SOD1 protein expression in those with ALS or at risk of developing ALS.

There is no cure for ALS. Management focuses on treating symptoms and providing supportive care, with the goal of improving quality of life and prolonging survival. There are few approved medications for the treatment of ALS. Tofersen (Qalsody) is an antisense oligonucleotide that was approved for medical use in the United States in April 2023, for the treatment of SOD1-associated ALS. To date, studies show a modest improvement in disease progression in ALS patients and a reduction in SOD1 protein levels following administration of Tofersen. However, treatments that can more potently delay disease progression and improve function in ALS patients are needed. Accordingly, there remains an unmet need for new therapeutic approaches for ALS.

### SUMMARY

The disclosure provides an RNA-targeting nucleic acid molecule comprising a small nuclear RNA (snRNA), wherein the snRNA comprises a targeting sequence that binds a SOD1 RNA sequence.

In some embodiments, the SOD1RNA sequence is an exon 2 sequence. In some aspects, the SOD1 targeting sequence comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 1-37.

In some embodiments, the snRNA comprises a stem loop (SL). In some aspects, the SL comprises one or more nucleic acid sequences set forth in any one of SEQ ID NOs: 51-91. In some embodiments, the SL comprises one or more nucleic acid sequences that are each, independently, at least 65%, 70%, 75%, 80%, 85%, 90%, 95%, 96%, 97%, 98%, 99% or 100% (or any percentage in between) identical to the nucleic acid sequence set forth in any one of SEQ ID NOs: 51-91.

The disclosure provides an RNA-targeting nucleic acid molecule comprising a small nuclear RNA (snRNA), wherein the snRNA comprises a targeting sequence that binds a SOD1 RNA sequence and a SL comprising the nucleic acid sequence set forth in any one of SEQ ID NOs: 51-91.

In some embodiments, the SOD1RNA sequence is exon 2 or exon 3. In some embodiments, the SOD1 targeting sequence comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 1-37.

In some embodiments, the SOD1 RNA sequence is a pre-mRNA or mRNA sequence.

In some embodiments, the snRNA comprises an Sm binding domain (SmBD). In some embodiments, the SmBD is a U1, U2, U4, or U5 SmBD. In some aspects, the SmBD comprises a nucleic acid sequence set forth in any one of SEQ ID NOs: 127 or 128.

In some embodiments, the snRNA comprises a 5′ interaction stabilizer domain (5′ISD).

In some aspects, the 5′ISD comprises the nucleotide sequence ggagt, cctct, ggaggt, cctcct, agccag, ggaag, gaagaag, gttg, ccgaa, taaggag, gaag, or ggctt.

The disclosure provides a vector comprising one or more RNA-targeting nucleic acid molecules of any embodiment of the disclosure. In some aspects, the vector is an adeno-associated virus (AAV) vector.

In some embodiments, the snRNA is operably linked to a promoter. In some aspects, the snRNA is operably linked to a U7 promoter or a U1 promoter. In some aspects, the snRNA is operably linked to a downstream terminator (DT). In some aspects, the snRNA is operably linked to a U7 downstream terminator or a U1 downstream terminator.

In some embodiments, the vector comprises at least one, at least two, at least three, at least four, or at least five snRNA.

In some embodiments, the least one, at least two, at least three, at least four, or at least five snRNA that each target the same target RNA sequence.

In some embodiments, each snRNA is separated by a buffer sequence. In some aspects, the buffer sequence comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one SEQ ID NOs: 118-124.

In some embodiments, the vector comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 38-50.

The disclosure provides a SOD1 RNA-targeting nucleic acid molecule comprising a targeting sequence set forth in any embodiment of the disclosure.

The disclosure provides a polynucleotide or vector comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 38-50.

The disclosure provides a recombinant AAV (rAAV) comprising: (a) an AAV capsid comprising an AAV capsid protein; and (b) a vector genome comprising a sequence encoding the RNA-targeting nucleic acid molecule of any embodiment of the disclosure, the SOD1 RNA-targeting nucleic acid molecule of any embodiment of the disclosure or the polynucleotide of any embodiment of the disclosure.

In some embodiments, the vector genome further comprises a 5′ inverted terminal repeat (ITR) sequence and a 3′ ITR. In some embodiments, the vector genome comprises, in the 5′ to 3′ direction, a 5′ ITR sequence, the snRNA or the RNA-targeting nucleic acid molecule, and a 3′ ITR sequence.

The disclosure provides a recombinant AAV (rAAV) comprising: (a) an AAV capsid comprising an AAV capsid protein; and (b) a vector genome comprising the polynucleotide of any embodiment of the disclosure.

In some embodiments, the AAV capsid comprises an AAV capsid protein of an AAV serotype selected from the group consisting of: AAV1, AAV2, AAV3, AAV4, AAV5, AAV6, AAV7, AAV8, AAV9, AAV10, AAVrh10, AAV11, AAV12, and variants thereof.

In some embodiments, the vector genome is single-stranded or self-complementary.

In some embodiments, the rAAV is replication incompetent.

The disclosure provides a pharmaceutical composition comprising the RNA-targeting nucleic acid molecule of any embodiment of the disclosure, the vector of any embodiment of the disclosure, the SOD1 RNA-targeting nucleic acid molecule of any embodiment of the disclosure, the polynucleotide or vector of any embodiment of the disclosure, or the rAAV of any embodiment of the disclosure.

The disclosure provides a method of targeting one or more target RNAs of interest and exon-skipping the one or more target RNAs, comprising contacting the RNA-targeting nucleic acid molecule of any embodiment of the disclosure, the vector of any embodiment of the disclosure, the SOD1 RNA-targeting nucleic acid molecule of any embodiment of the disclosure, the polynucleotide or vector of any embodiment of the disclosure, or the rAAV of any embodiment of the disclosure, with a cell comprising the one or more target RNAs.

The disclosure provides a method of treating a disease or disorder in a subject in need thereof comprising administering to the subject the RNA-targeting nucleic acid molecule of any embodiment of the disclosure, the vector of any embodiment of the disclosure, the SOD1 RNA-targeting nucleic acid molecule of any embodiment of the disclosure, the polynucleotide or vector of any embodiment of the disclosure, or the rAAV of any embodiment of the disclosure.

In some embodiments, the disease or disorder is Amyotrophic Lateral Sclerosis (ALS).

The disclosure provides use of the RNA-targeting nucleic acid molecule of any embodiment of the disclosure, the vector of any embodiment of the disclosure, the SOD1 RNA-targeting nucleic acid molecule of any embodiment of the disclosure, the polynucleotide or vector of any embodiment of the disclosure, or the rAAV of any embodiment of the disclosure for targeting one or more target RNAs of interest and exon-skipping the one or more target RNAs.

In some embodiments, the one or more target RNAs are in a cell.

The disclosure provides a use of a snRNA or RNA-targeting nucleic acid molecule as disclosed herein for targeting one or more target RNAs of interest and exon-skipping the one or more target RNAs.

The disclosure provides a use of a snRNA, RNA-targeting nucleic acid molecule, vector, polynucleotide, rAAV, or pharmaceutical composition as disclosure herein for treating a disease or disorder in a subject.

The disclosure provides use of the RNA-targeting nucleic acid molecule of any embodiment of the disclosure, the vector of any embodiment of the disclosure, the SOD1 RNA-targeting nucleic acid molecule of any embodiment of the disclosure, the polynucleotide or vector of any embodiment of the disclosure, or the rAAV of any embodiment of the disclosure for treating a disease or disorder in a subject.

In some embodiments, the administration is systemic, intravenous, or intracerebroventricular.

In some embodiments, the one or more target RNAs are comprised in a cell.

The disclosure provides a kit comprising the snRNA, RNA-targeting nucleic acid molecule, polynucleotide, vector, rAAV or pharmaceutical composition of the disclosure.

The disclosure provides use of the snRNA, RNA-targeting nucleic acid molecule, polynucleotide, vector, rAAV or pharmaceutical composition of the disclosure in the manufacture of a medicament for treating a disease or disorder in a subject.

In some embodiments, the SOD1 RNA sequence is a wild-type SOD1 RNA sequence or a mutant SOD1 RNA sequence.

In some embodiments, the SOD1 mutation comprises at least one of A4V, H46R, or G93A.

The disclosure provides a recombinant AAV (rAAV) comprising: a nucleic acid sequence encoding a first small nuclear RNA (snRNA) molecule, wherein the first snRNA comprises a targeting sequence that binds a target SOD1 RNA sequence; and a nucleic acid sequence encoding a hardened SOD1 nucleic acid sequence encoding a SOD1 polypeptide.

In some embodiments, the rAAV further comprises a nucleic acid sequence encoding a second snRNA molecule comprising a targeting sequence that binds a target SOD1 RNA sequence.

In some embodiments, the rAAV further comprises a nucleic acid sequence encoding a third snRNA molecule comprising a targeting sequence that binds a target SOD1 RNA sequence, and optionally a fourth snRNA molecule comprising a targeting sequence that binds a target SOD1 RNA sequence.

In some embodiments, the nucleic acid sequence encoding the hardened SOD1 nucleic acid sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 131.

In some embodiments, the first snRNA is operably linked to a first promoter and the second snRNA is operably linked to a second promoter.

In some embodiments, the hardened SOD1 nucleic acid sequence is operably linked to a third promoter.

In some embodiments, the first promoter and the second promoter are the same promoter.

In some embodiments, the first promoter and the second promoter are different promoters.

In some embodiments, the first promoter is a U1, U2, U4 U5, U6, or U7 promoter.

In some embodiments, the second promoter is a U1, U2, U4 U5, U6, or U7 promoter.

The rAAV of any embodiment of disclosure, wherein the first promoter and the second promoter is a human promoter or a murine promoter.

In some embodiments, the third promoter is a pJET promoter.

In some embodiments, the first snRNA molecule, and optionally the second snRNA molecule, third snRNA molecule, and/or fourth snRNA molecule each do not bind the nucleic acid sequence encoding the hardened SOD1 nucleic acid sequence.

In some embodiments, the target SOD1 RNA sequence comprises a mutation.

In some embodiments, the vector comprises: a nucleic acid sequence encoding a first inverted terminal repeat (ITR) sequence; a nucleic acid sequence encoding a first promoter sequence; a nucleic acid sequence encoding a first snRNA sequence wherein the first snRNA comprises a targeting sequence that binds a target SOD1 RNA sequence; a nucleic acid sequence encoding a second promoter sequence; a nucleic acid sequence encoding a second snRNA sequence wherein the second snRNA comprises a targeting sequence that binds a target SOD1 RNA sequence; a nucleic acid sequence encoding a third promoter sequence; a nucleic acid sequence encoding a hardened SOD1 nucleic acid sequence encoding a SOD1 polypeptide; a nucleic acid sequence encoding a polyA sequence; a nucleic acid sequence encoding a second ITR sequence.

In some embodiments, the nucleic acid sequence encoding a hardened SOD1 nucleic acid sequence is expressed from the sense strand of the vector.

In some embodiments, the nucleic acid sequence encoding a hardened SOD1 nucleic acid sequence is expressed from the antisense strand of the vector.

The disclosure provides a method of treating Amyotrophic Lateral Sclerosis (ALS) in a subject in need thereof comprising administering to the subject the rAAV of any embodiment of disclosure.

In some embodiments, the administration is systemic, intravenous, or intracerebroventricular.

The disclosure provides a kit, comprising the RNA-targeting nucleic acid molecule of any embodiment of disclosure, the vector of any embodiment of disclosure, the SOD1 RNA-targeting nucleic acid molecule of any embodiment of disclosure, the polynucleotide or vector of any embodiment of disclosure, or the rAAV of any embodiment of disclosure, and instructions for use.

The disclosure provides a use of the RNA-targeting nucleic acid molecule of any embodiment of disclosure, the vector of any embodiment of disclosure, the SOD1 RNA-targeting nucleic acid molecule of any embodiment of disclosure, the polynucleotide or vector of any embodiment of disclosure, or the rAAV of any embodiment of disclosure in the manufacture of a medicament for treating a disease or disorder in a subject.

### BRIEF DESCRIPTION OF THE DRAWINGS

FIG. 1A depicts the mechanism of action for mutant toxic SOD1 knockdown using engineered U7 snRNAs. U7 snRNAs were engineered to bind splicing regulatory sequences (acceptor and donor sites or splicing enhancer sequences) to promote skipping of a constitutive exon (SOD1 exon 2 or 3) which leads to a frameshift and generation of a premature stop codon (PTC) to promote RNA degradation by the nonsense mediated decay (NMD) quality control system.

FIG. 1B shows a tapestation image of the RT-PCR products after U7 snRNA treatments using single U7 spacers (expressed by pcDNA-U7 snRNA single cassette under the control of hU7 promoter), showing the SOD1 exon 1 to 4 PCR product (non-skipped band, top amplicon), and the skipped products (skipping of exon 3, middle amplicon; or skipping of exon 2, bottom amplicon). Untreated (U) cells and NT (non-targeting) U7 snRNA were used as negative controls.

FIG. 1C depicts the qRT-PCR results for SOD1 mRNA expression post-treatment with multiple U7 snRNAs with single spacers (z1 to z12). The y-axis shows levels of human endogenous SOD1 RNA expression in HEK293T cells normalized to GAPDH reference gene and the non-targeting spacer (NT) and the x axis depicts the spacer used.

FIG. 2A shows a tapestation image of the RT-PCR products after U7 snRNA treatments using single or fusion U7 spacers expressed under the human U7 (hU7) or mouse U1 (mU1) promoters in HEK293T cells 48 h post-transfection. The gel image shows the SOD1 exon 1 to 4 PCR product (non-skipped band, top), and the skipped products (without exon 2, bottom band). NT (non-targeting U7 snRNA was used as a negative control).

FIG. 2B depicts the qRT-PCR results for endogenous human SOD1 mRNA expression post-treatment with multiple single and fusion U7 snRNAs. The y-axis shows levels of SOD1 RNA expression normalized to the GAPDH reference gene and the non-targeting spacer (NT), and the x-axis depicts the spacer used.

FIG. 2C shows quantification of the levels of SOD1 endogenous protein in HEK-293T cells treated with U7 snRNAs 48 h post-transfection. SOD1 protein expression was normalized to GAPDH loading control protein levels and normalized to non-targeting control (NT). The y-axis shows levels of SOD1 endogenous protein expression normalized to GAPDH protein and x-axis depicts the construct used.

FIG. 3A shows a tapestation image of the RT-PCR products after U7 snRNA treatments using unitary AAV vectors containing dual cassettes expressing fusion U7 spacers expressed under mouse U7 and mouse U1 promoters 48 h post-transfection in HEK293T cells. The gel image shows the SOD1 exon 1 to 4 PCR product (non-skipped band, top), and the skipped product (without exon 2, bottom band). NT (non-targeting U7 was used as a negative control).

FIG. 3B depicts the qRT-PCR results for SOD1 mRNA expression post-treatment with unitary AAVs expressing dual fusion U7 snRNA cassettes. The y-axis shows levels of SOD1 RNA expression normalized to the GAPDH reference gene and NT control, and the x-axis depicts the construct used.

FIG. 3C shows quantification of the levels of SOD1 endogenous protein in HEK-293T cells treated with U7 snRNAs 72 h post-transfection. SOD1 protein expression was normalized to GAPDH loading control protein levels and normalized to non-targeting control (NT).

FIG. 4A depicts droplet digital PCR (ddPCR) results for SOD1 RNA expression 7 days post-transduction with 5E5, 2E6 or 5E6 multiplicity of infection (MOI) of AAV9 expressing dual U7 snRNA cassettes (3 different fusion constructs, A04054, Fz9+z2; A04055, Fz9+z3; and A05204, Fz3+z10) in motor neurons with SOD1 ALS mutation A4V. The y-axis shows copies of SOD1 RNA per 1000 copies of GAPDH reference gene and the x-axis depicts the construct used.

FIG. 4B shows quantification of U7 snRNA expression. The y-axis shows copies of U7 snRNA per ng of total RNA and the x-axis depicts the construct used. AAV refers to AAV9 null (empty capsid) used as negative control and UNT to untreated cells.

FIGS. 5A-5E show study design and results from in vivo studies of wild type (WT) C57Bl/6 mice treated with scAAV9-U7 snRNA targeting mouse SOD1 for knockdown delivered intrastriatal and harvested 4 weeks post-injection. FIG. 5A shows a table representing the study design of WT mice injected unilaterally with 4E9 vector genomes (vg)/animal of scAAV9 (containing 2×U7 snRNA cassettes). Untreated unilateral striatum was used as negative control. FIG. 5B is a diagram showing the injection site of treated vs untreated contralateral striatal sides. FIG. 5C is a tapestation image for striatal samples of the RT-PCR products after U7 snRNA treatments using 2 lead constructs A05323 and A05325 (both containing 2×U7 fusion spacer cassettes targeting mouse SOD1), showing the SOD1 excluded products (without exon 2 or without exon 2 and 3; U refers to untreated samples and T snRNA treated; individual numbers represent mice ID). FIG. 5D shows quantification of the levels of mouse SOD1 mRNA 4 weeks post AAV9 delivery. The y-axis depicts fold change of SOD1 mRNA relative to untreated contralateral controls and normalized to ATP5b reference gene, and the x-axis depicts the treatment. FIG. 5E shows a representative gel image of mouse SOD1 protein expression in untreated striatum (U) or treated (T) with A05323-dual snRNA construct (M refers to marker or ladder). FIG. 5F Shows quantification of the levels of mouse SOD1 endogenous protein in untreated striatum (U) or treated (T) with A05323.

FIGS. 6A-6G show efficacy of SOD1 knockdown and replace construct A05433 transfected in HEK293T cells and transduced as scAAV9 in SOD1 A4V motor neurons. FIG. 6A is a diagram of the AAV plasmid design used for SOD1 knockdown and replacement strategy, expressing 2 snRNA cassettes (under mouse U7 and mouse U1 promoters), and the expression of hardened SOD1 (with mutated nucleotides so snRNA is unable to target) under the control of the synthetic pJET promoter being expressed from the antisense strand. FIG. 6B shows a tapestation image of the RT-PCR products after U7 snRNA treatments with the SOD1 exon 1 to 4 PCR product (non-skipped band, top), and the skipped products (skipping of exon 2, middle band; or skipping of exon 2 and 3, bottom band); NT (non-targeting U7 was used as a negative control), dual fusion U7 snRNA (A05204), dual fusion U7 snRNA with wild type SOD1 replacement (A05433), and untreated control (UNT). FIG. 6C shows quantification of human endogenous SOD1 mRNA 48 h post-transfection in HEK293T cells. The y-axis shows levels of hSOD1 RNA expression normalized to GAPDH reference gene and NT control, and the x-axis depicts the construct used. FIG. 6D shows a Western blot of SOD1 protein with GAPDH as loading control 48 h post-transfection in HEK293T cells. Top band is referent to SOD1_HA tag replacement, and bottom band represents endogenous SOD1 protein levels. FIG. 6E shows ddPCR results for SOD1 RNA expression 7 days post-transduction with 1E6 or 2E6 MOI of scAAV9 expressing dual U7 snRNA cassettes (A05204, z3/z10; A05433, z3/z10+SOD1 replacement cassette) in motor neurons with SOD1 ALS mutation A4V. The y-axis shows copies of SOD1 RNA per 1000 copies of GAPDH reference gene and the x-axis depicts the construct used and MOI. FIG. 6F shows ddPCR results for codon optimized SOD1 replacement transcript expression using specific primers for the replacement sequence. The y-axis shows copies of SOD1 replacement RNA per 1000 copies of GAPDH reference gene and the x-axis depicts the construct used and the MOI. FIG. 6G shows SOD1 enzymatic activity in motor neurons with SOD1 ALS mutation A4V 7 days post transduction with AAV9-U7 snRNA−/+replacement. UNT refers to untreated cells, and AAV null refers to AAV9 empty capsid control.

### DETAILED DESCRIPTION

The disclosure provides gene therapy compositions comprising a therapeutic RNA-targeting platform comprised of short nuclear RNA (snRNA) targeting precursor mRNA (pre-mRNA) or mRNA sequences encoding superoxide dismutase 1 (SOD1). The targeted pre-mRNA or mRNA sequences can include exonic regions of SOD1 and/or splicing regulatory sequences of SOD1.

Disclosed herein are compositions comprising nucleic acid molecules, and vectors comprising the snRNA construct or constructs targeting SOD1. The snRNA molecules of the disclosure can be non-natural, modified and/or engineered snRNA (esnRNA). In some embodiments, snRNA or engineered snRNA (esnRNA) targeting SOD1 of the disclosure comprise a mutated snRNA stem loop. In some embodiments, snRNA targeting SOD1 of the disclosure comprises a native stem loop.

Small nuclear ribonucleic acids (snRNAs) are essential components of small nuclear ribonucleoprotein complexes (snRNPs) which, when assembled with additional proteins, form the large ribonucleoprotein complex known as the spliceosome, the cellular machinery appointed to mediate the entire mRNA maturation process. The spliceosome is responsible for precursor mRNA splicing, which is the process that removes introns from RNA transcripts before protein production. An individual snRNA is generally about 250 nucleotides or less in size. For example, U1 snRNA is 164 nucleotides in length and is encoded by genes that occur in several copies within the human genome. U1 snRNA represents the ribonucleic component of the nuclear particle U1 snRNP. The U1 snRNA has a stem and loop tridimensional structure and within the 5′ region there is a single-stranded sequence, generally about 9 nucleotides in length, capable of binding by complementary base pairing to the splicing donor site on the pre-mRNA molecule. (Horowitz et al., 1994, Trends Genet., 10(3):100-6.) The various spliceosomal snRNAs have been designated as U1, U2, U4, U5, U6, U4ATAC, U6ATAC, U7, U11 and U12, due to the generous amount of uridylic acid they contain. (Mattaj et al., 1993, FASEB J, 15, 7:47-53.)

snRNA systems can be used for treating toxic mutations. For example, antisense oligonucleotides that interfere with splice sites and regulatory elements within an exon containing toxic mutations can induce skipping of specific exons at the pre-RNA level. Such antisense sequences can be packaged in an snRNA sequence delivered using viral vectors carrying a nucleic acid sequence from which the snRNA can be transcribed. U7 snRNA is endogenously involved in histone pre-mRNA 3′-end processing but can be converted into a versatile tool for splicing modulation by a small change in the binding site for Sm/Lsm proteins.

Most U-rich snRNPs are complexes that mediate the splicing of pre-mRNAs. U7 snRNP is an exception. U7 is not involved in splicing but rather is a key factor in the unique 3′-end processing of replication-dependent histone mRNAs. By modifying the U7 snRNA histone binding sequence and the Sm motif, U7 can no longer be involved in processing the histone pre-mRNA and instead targets pre-mRNAs or mRNA for blocking or splicing modulation. In this manner, U7 snRNA can be used as an effective gene therapy platform. The U7 snRNA platform also has the additional advantages of being a compact size, having the capability to accumulate in the nucleus without causing cellular toxicity, and possesses little to no immunoreactivity. (Gadgil et al., 2021, J Gene Med, 23(4): e3321.). The U7 snRNA platform is described in more detail in International Patent Application Publication No. WO 2023/168458, which is incorporated herein by reference in its entirety.

In some aspects disclosed herein are esnRNA comprising a stem loop (SL). Compensatory modifications made to the native stem loop sequence can create an engineered stem loop (eSL) which more effectively communicates (folds and anneals) with the snRNA interaction stabilization domain (ISD) compared to the native stem loop sequence, which in turn creates an snRNA platform with increased stability. U7 snRNAs have been previously shown to be programmable to modulate mRNAs. Disclosed herein are programmed engineered snRNA improvements which are capable of being used as a gene therapy tool.

snRNA systems disclosed herein are configured to bind target SOD1 RNA sequences to modulate RNA splicing which can lead to single or multiple exon skipping, or exon inclusion of targeted sequences of the SOD1 RNA. SOD1-targeting snRNA are configured to bind to SOD1 pre-mRNA molecules at sites that regulate RNA splicing. Splicing regulatory sites can include splice acceptor sequences, splice donor sequences, and exon splice enhancer sequences. snRNA sequences of the disclosure can induce exon skipping (of single or multiple exons) of targeted exonic sequences.

SOD1 targeting snRNA of the disclosure can be configured to target any SOD1 variant, including wild-type SOD1 or mutant forms of SOD1. In some aspects, snRNA of the disclosure target wild-type SOD1. In some aspects, snRNA of the disclosure target SOD1 mutants. In some embodiments, SOD1 mutants can comprise SOD1 A4V, H46R, G93A, or any other SOD1 mutant. In some aspects, the SOD1 mutation is numbered in reference to the SOD1 amino acid sequence set forth SEQ ID NO: 134. SOD1 mutations will be known to persons of ordinary skill in the art, and are described, for example, in databases such as fujisawagroup.github.io/SoDCoDweb/.

SOD1 targeting snRNA of the disclosure can be configured to induce skipping of one or more constitutive exons of SOD1 (WT and mutant), such as exon 2 or exon 3. Such exon skipping can be configured to introduce a premature termination codon (PTC) into the resulting spliced SOD1 mRNA molecule. Transcription of the mRNA comprising the PTC results in nonsense mediated decay of the RNA. Without wishing to be bound by theory, it is hypothesized that by promoting skipping of an exonic region of SOD1, such as exon 2 or exon 3, from the SOD1 pre-mRNA so as to introduce a PTC into the processed mRNA using the U7 snRNA platform, whereby SOD1 RNA and protein levels are reduced.

In some aspects, compositions of the disclosure also encode a sequence encoding wild-type SOD1. In some aspects, the wild-type SOD1 is “hardened” (modified at the nucleotide level), such that snRNA compositions of the disclosure cannot bind the expressed wild-type/hardened SOD1 replacement. Accordingly, the subject will see the toxic or mutant SOD1 expression levels reduced and expression of the replacement hardened/wild-type SOD1 is increased.

In one embodiment, the snRNAs are human snRNAs. In another embodiment, the snRNAs are mouse snRNAs. In another embodiment, the snRNAs are of any species. In another embodiment, the snRNAs are a combination of human and mouse snRNAs. In one embodiment, the U7 snRNA is a human U7 snRNA or a mouse U7 snRNA. In another embodiment disclosed herein, snRNA is chimeric, i.e. comprises varying types of snRNAs (U1-U12, etc.) by combining domains of endogenous snRNAs to fine tune stabilization of the platform and/or to reduce off-target effects. For example, in one embodiment, the snRNA compositions of the disclosure comprise a combination of human or mouse U7 snRNA and human or mouse U1 snRNA components.

Additional elements that can tune the processing and abundance of the RNA can be further engineered into the snRNAs or esnRNAs comprising SLs. In one embodiment, additional elements that can tune the processing, stability, and abundance of the esnRNA can be further engineered into the esnRNAs at the 5′ or 3′ ends. In another embodiment, such elements may include but are not limited to stem loops, hairpins, G-C clamps, kissing loops, triplexes, quadruplexes, and protein binding sites.

The snRNA platform and portions thereof can be used in a therapeutic setting and context so long as a suitable spacer(s) or target sequence (s) TS(s) is included in the design of the therapeutic composition. In certain embodiments, a therapeutic snRNA composition is used to treat a disease associated with dysregulated, mutated, or non-functional SOD1. In some aspects, the disease or disorder is amyotrophic lateral sclerosis.

### Targeting Sequences

The snRNA systems can be programmed to comprise a targeting sequence (TS) (also termed “spacer”) that targets an RNA of interest. The snRNA systems can be programmed with one or more targeting sequences targeting one or more RNAs of interest. In some aspects, the targeting sequence is a 5′ targeting sequence (5′TS) that targets one or more RNAs of interest. In this context, 5′ is in reference to the snRNA insert's 5′ end and not necessarily to the overall vector configuration comprising the snRNA insert or inserts. The TS can be located in or near the 5′ end of the snRNA. In an alternative embodiment, the targeting sequence(s) (TS) can be located in or near a 3′ position in the snRNA construct, thereby generating a 3′ targeting sequence (3′ TS), particularly if the snRNA construct is not a U7-based snRNA.

Targeting sequences of the disclosure, including 5′ TS, and 3′TS can be between about 1 and about 200 nucleotides in length. In some aspects, targeting sequences of the disclosure are between about 10 and about 150 nucleotides in length. In some aspects, targeting sequences of the disclosure are between about 10 and about 100 nucleotides in length. In some aspects, targeting sequences of the disclosure are between about 20 and about 60 nucleotides in length. In some aspects, targeting sequences of the disclosure are at least about 10, 20, 30, 40, 50, 60, or about 70 nucleotides in length.

snRNA compositions of the disclosure can comprise more than one targeting sequences, wherein each targeting sequence binds a distinct RNA sequence. In some aspects, snRNA of the disclosure comprise a fusion targeting sequence. In some aspects, a fusion targeting sequence is a nucleic acid sequence comprising two or more targeting sequences directly connected to each other, or connected by one or more linker nucleic acid sequences, or a combination thereof. In some embodiments, each targeting sequence binds a distinct RNA sequence. In some embodiments, the distinct RNA sequences are within the same target sequence, i.e. a SOD1 RNA sequence as described herein. In some embodiments, each targeting sequence binds a different target RNA sequence.

In one example, U7 snRNA can be programmed by replacing the histone mRNA binding sequence with a sequence complementary to a target of interest. In some aspects, snRNA systems of the disclosure bind a target mRNA or pre-mRNA sequence of interest. The exemplary snRNA systems shown herein lead to exon skipping (for treating ALS, e.g., SOD1 exon 2 skipping).

In some embodiments, snRNAs of the disclosure target a pre-mRNA or mRNA sequence encoding the SOD1 protein. SOD1 is a gene encoding the protein superoxide dismutase 1 (SOD1). Mutations in SOD1 are associated with ALS. In some embodiments, the SOD1 RNA sequence targeted by snRNA compositions of the disclosure can be any exonic or intronic SOD1 RNA sequence. In some embodiments, the SOD1 RNA sequence targeted by snRNA compositions of the disclosure is an exon 2 or exon 3 SOD1 RNA sequence. In some embodiments, the SOD1 RNA sequence targeted by snRNA compositions of the disclosure is a human exon 2 or exon 3 SOD1 RNA sequence. In some embodiments, the SOD1 RNA sequence targeted by snRNA compositions of the disclosure is a murine exon 2 or exon 3 SOD1 RNA sequence. In some embodiments, the SOD1 RNA sequence targeted by the snRNA of the disclosure is a splice acceptor sequence, a splice donor sequence, or an exon splice enhancer sequence. In some embodiments, the snRNAs of the disclosure target any combination of a splice acceptor sequence, a splice donor sequence, and an exon splice enhancer sequence.

In some embodiments, the SOD1 RNA sequence targeted by snRNA compositions of the disclosure is a mutant SOD1. In some aspects, the SOD1 mutation is A4V, H46R, or G93A. Any other ALS-associated SOD1 mutation is also contemplated.

In some embodiments, the nucleic acid sequence encoding wild-type human SOD1 mRNA comprises or consists of SEQ ID NO: 133. In some embodiments, the nucleic acid sequence encoding wild-type human SOD1 mRNA comprises a sequence that is at least 65%, 70%, 75%, 80%, 85%, 90%, 95%, 96%, 97%, 98%, or 99% identical to the sequence set forth in SEQ ID NO: 133.

Targeting sequences that bind human SOD1 exon 2 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 1, which follows:

TABLE 1
SOD1 exon 2 targeting sequences

| | | | SEQ |
|---|---|---|---|
| Plasmid | | | ID |
| UID | Spacer | SOD1 exon 2 single spacers | NO |
| P04796 | z1 | CTGGTCCATTACTTTCCTTT | 1 |
| P04797 | z2 | CACACCTTCACTGGTCCATTACTTTCCTTT | 2 |
| P04798 | z3 | TTCCCCACACCTTCACTGGTCCATTA | 3 |
| P04799 | z4 | CCCACACCTTCACTGGTCCA | 4 |
| P04800 | z5 | TTCAGTCAGTCCTTTAATGCTTCCCC | 5 |
| P04801 | z6 | CCATGCAGGCCTTCAGTCAGTCCTTTAATG | 6 |
| P04802 | z7 | GAATCCATGCAGGCCTTCAGTCAGTCCTTT | 7 |
| P04803 | z8 | CCATGCAGGCCTTCAGTCAG | 8 |
| P04804 | z9 | GCTGTATTATCTCCAAACTCATGAAC | 9 |
| P04805 | z10 | ACCTGCTGTATTATCTCCAAACTCATGAAC | 10 |
| none | z11 | CACACCTTCACTGGTCCATTACTTTCCTTTAAGAAA | 13 |
| none | z12 | aCACCCACCTGCTGTATTATCTCCAAACTCATGAAC | 14 |
| none | z13 | TCCCCACACCTTCACTGGTCCATTACTTTCCTTTAA | 15 |
| none | z14 | CCACCTGCTGTATTATCTCCAAACTCATGAACATGG | 16 |

In some embodiments, the targeting sequence that binds human SOD1 exon 2 comprises the sequence set forth in SEQ ID NO: 1. In some embodiments, the targeting sequence that binds human SOD1 exon 2 comprises the sequence set forth in SEQ ID NO: 2. In some embodiments, the targeting sequence that binds human SOD1 exon 2 comprises the sequence set forth in SEQ ID NO: 3. In some embodiments, the targeting sequence that binds human SOD1 exon 2 comprises the sequence set forth in SEQ ID NO: 4. In some embodiments, the targeting sequence that binds human SOD1 exon 2 comprises the sequence set forth in SEQ ID NO: 5. In some embodiments, the targeting sequence that binds human SOD1 exon 2 comprises the sequence set forth in SEQ ID NO: 6. In some embodiments, the targeting sequence that binds human SOD1 exon 2 comprises the sequence set forth in SEQ ID NO: 7. In some embodiments, the targeting sequence that binds human SOD1 exon 2 comprises the sequence set forth in SEQ ID NO: 8. In some embodiments, the targeting sequence that binds human SOD1 exon 2 comprises the sequence set forth in SEQ ID NO: 9. In some embodiments, the targeting sequence that binds human SOD1 exon 2 comprises the sequence set forth in SEQ ID NO: 10. In some embodiments, the targeting sequence that binds human SOD1 exon 2 comprises the sequence set forth in SEQ ID NO: 13. In some embodiments, the targeting sequence that binds human SOD1 exon 2 comprises the sequence set forth in SEQ ID NO: 14. In some embodiments, the targeting sequence that binds human SOD1 exon 2 comprises the sequence set forth in SEQ ID NO: 15. In some embodiments, the targeting sequence that binds human SOD1 exon 2 comprises the sequence set forth in SEQ ID NO: 16.

In some embodiments, the targeting sequence comprises, consists of, or consists essentially of the sequence set forth in any one of SEQ ID NOs: 1-16, or a sequence having 1, 2, 3, or 4 substitutions, insertions or deletions relative thereto.

Targeting sequences that bind human SOD1 exon 3 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 1, which follows:

TABLE 2
SOD1 exon 3 targeting sequences

| | | | SEQ |
|---|---|---|---|
| Plasmid | | | ID |
| UID | Spacer | SOD1 exon 2 single spacers | NO |
| P04806 | z1-E3 | TTCATCCTTTGGCCCACCGTGTTTTC | 11 |
| P04807 | z2-E3 | ACCTCTCTTCATCCTTTGGCCCACCGTG | 12 |
| | | TT | |

In some embodiments, the targeting sequence that binds human SOD1 exon 3 comprises the sequence set forth in SEQ ID NO: 11. In some embodiments, the target sequence that binds human SOD1 exon 3 comprises the sequence set forth in SEQ ID NO: 12.

The sequences set forth in Table 1 or Table 2 may be combined to generate fusion targeting sequences (sometimes referred to herein as “spacers”). Any first sequence set forth in Table 1 or Table 2 may combined with any second sequence set forth in in Table 1 or Table 2. Illustrative fusion spacers are set forth in Table 2. Targeting sequences that bind human SOD1 exon 2 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in Table 2, which follow.

TABLE 2
SOD1 Exon 2 Fusion Spacers-Targeting Sequences

| Plasmid | | | |
|---|---|---|---|
| UID | Spacer | SOD1 exon 2 Fusion spacers | SEQ ID NO |
| P05050 | z4/z8 | CCCACACCTTCACTGGTCCACCATGCAGGCC | 17 |
| | | TTCAGTCAG | |
| A04053 | z8/z4 | CCATGCAGGCCTTCAGTCAGCCCACACCTTC | 18 |
| | | ACTGGTCCA | |
| A05204 | z3/z10 | TTCCCCACACCTTCACTGGTCCATTAACCTGC | 19 |
| | | TGTATTATCTCCAAACTCATGAAC | |
| P05053 | z10/z3 | ACCTGCTGTATTATCTCCAAACTCATGAACTT | 20 |
| | | CCCCACACCTTCACTGGTCCATTA | |
| P05054 | z3/z7 | TTCCCCACACCTTCACTGGTCCATTAGAATCC | 21 |
| | | ATGCAGGCCTTCAGTCAGTCCTTT | |
| P05055 | z7/z3 | GAATCCATGCAGGCCTTCAGTCAGTCCTTTTT | 22 |
| | | CCCCACACCTTCACTGGTCCATTA | |
| P05096 | z7/z10 | GAATCCATGCAGGCCTTCAGTCAGTCCTTTAC | 23 |
| | | CTGCTGTATTATCTCCAAACTCATGAAC | |
| P05097 | z10/z7 | ACCTGCTGTATTATCTCCAAACTCATGAACG | 24 |
| | | AATCCATGCAGGCCTTCAGTCAGTCCTTT | |
| P05179 | z2/z9 | CACACCTTCACTGGTCCATTACTTTCCTTTGC | 25 |
| | | TGTATTATCTCCAAACTCATGAAC | |
| A04054 | z9/z2 | GCTGTATTATCTCCAAACTCATGAACCACAC | 26 |
| | | CTTCACTGGTCCATTACTTTCCTTT | |
| P05181 | z3/z9 | TTCCCCACACCTTCACTGGTCCATTAGCTGTA | 27 |
| | | TTATCTCCAAACTCATGAAC | |
| A04055 | z9/z3 | GCTGTATTATCTCCAAACTCATGAACTTCCCC | 28 |
| | | ACACCTTCACTGGTCCATTA | |
| A05207 | z11/z12 | CACACCTTCACTGGTCCATTACTTTCCTTTAA | 29 |
| | | GAAAACACCCACCTGCTGTATTATCTCCAAA | |
| | | CTCATGAAC | |
| A05208 | z12/z11 | ACACCCACCTGCTGTATTATCTCCAAACTCAT | 30 |
| | | GAACCACACCTTCACTGGTCCATTACTTTCCT | |
| | | TTAAGAAA | |
| A05209 | z13/z14 | TCCCCACACCTTCACTGGTCCATTACTTTCCT | 31 |
| | | TTAACCACCTGCTGTATTATCTCCAAACTCAT | |
| | | GAACATGG | |
| A05210 | z14/z13 | CCACCTGCTGTATTATCTCCAAACTCATGAAC | 32 |
| | | ATGGTCCCCACACCTTCACTGGTCCATTACTT | |
| | | TCCTTTAA | |
| A05205 | z2/z10 | CACACCTTCACTGGTCCATTACTTTCCTTTAC | 33 |
| | | CTGCTGTATTATCTCCAAACTCATGAAC | |
| A05206 | z10/z2 | ACCTGCTGTATTATCTCCAAACTCATGAACCA | 34 |
| | | CACCTTCACTGGTCCATTACTTTCCTTT | |

In some embodiments, the targeting sequence that binds human SOD1 exon 2 comprises the sequence set forth in SEQ ID NO: 17. In some embodiments, the targeting sequence that binds human SOD1 exon 2 comprises the sequence set forth in SEQ ID NO: 18. In some embodiments, the targeting sequence that binds human SOD1 exon 2 comprises the sequence set forth in SEQ ID NO: 19. In some embodiments, the targeting sequence that binds human SOD1 exon 2 comprises the sequence set forth in SEQ ID NO: 20. In some embodiments, the targeting sequence that binds human SOD1 exon 2 comprises the sequence set forth in SEQ ID NO: 21. In some embodiments, the targeting sequence that binds human SOD1 exon 2 comprises the sequence set forth in SEQ ID NO: 22. In some embodiments, the targeting sequence that binds human SOD1 exon 2 comprises the sequence set forth in SEQ ID NO: 23. In some embodiments, the targeting sequence that binds human SOD1 exon 2 comprises the sequence set forth in SEQ ID NO: 24. In some embodiments, the targeting sequence that binds human SOD1 exon 2 comprises the sequence set forth in SEQ ID NO: 25. In some embodiments, the targeting sequence that binds human SOD1 exon 2 comprises the sequence set forth in SEQ ID NO: 26. In some embodiments, the targeting sequence that binds human SOD1 exon 2 comprises the sequence set forth in SEQ ID NO: 27. In some embodiments, the targeting sequence that binds human SOD1 exon 2 comprises the sequence set forth in SEQ ID NO: 28. In some embodiments, the targeting sequence that binds human SOD1 exon 2 comprises the sequence set forth in SEQ ID NO: 29. In some embodiments, the targeting sequence that binds human SOD1 exon 2 comprises the sequence set forth in SEQ ID NO: 30. In some embodiments, the targeting sequence that binds human SOD1 exon 2 comprises the sequence set forth in SEQ ID NO: 31. In some embodiments, the targeting sequence that binds human SOD1 exon 2 comprises the sequence set forth in SEQ ID NO: 32. In some embodiments, the targeting sequence that binds human SOD1 exon 2 comprises the sequence set forth in SEQ ID NO: 33. In some embodiments, the targeting sequence that binds human SOD1 exon 2 comprises the sequence set forth in SEQ ID NO: 34.

In some embodiments, the targeting sequence comprises, consists of, or consists essentially of the sequence set forth in any one of SEQ ID NOs: 17-34, or a sequence having 1, 2, 3, or 4 substitutions, insertions or deletions relative thereto.

Illustrative murine SOD1 fusion spacers are set forth in Table 4. Targeting sequences that bind murine SOD1 exon 2 can comprise, consist essentially of, or consist of a nucleic acid sequence at least 65%, 70%, 75%, 80%, 85%, 90%, 95%, 96%, 97%, 98%, 99% or 100% (or any percentage in between) identical to one or more of the following nucleotide sequences set forth in 4, which follow.

TABLE 4
SOD1 Exon 2 Murine Fusion Spacers - Targeting Sequences

| Plasmid | | | |
|---|---|---|---|
| UID | Spacer | SOD1 exon 2 Fusion spacers | SEQ ID NO |
| A05323 | Mus | tgTGTATTgTCcCCAtACTgATGgACgTCCtgACAa | 35 |
| | z9/z3 | CacaACTGGTtCAccg | |
| A05325 | Mus | ACCTtgTGTATTgTCcCCAtACTgATGgACgACAa | 36 |
| | z10/z2 | CacaACTGGTtCAccgCTTgCCTTg | |
| A05433 | z3/z10 | TTCCCCACACCTTCACTGGTCCATTAACCTGC | 37 |
| | | TGTATTATCTCCAAACTCATGAAC | |

In some embodiments, the targeting sequence that binds murine SOD1 exon 2 comprises the sequence set forth in SEQ ID NO: 35. In some embodiments, the targeting sequence that binds murine SOD1 exon 2 comprises the sequence set forth in SEQ ID NO: 36. In some embodiments, the targeting sequence that binds murine SOD1 exon 2 comprises the sequence set forth in SEQ ID NO: 37.

In some embodiments, the targeting sequence comprises, consists of, or consists essentially of the sequence set forth in any one of SEQ ID NOs: 35-37, or a sequence having 1, 2, 3, or 4 substitutions, insertions or deletions relative thereto.

### Stem Loops

The engineered snRNA (esnRNA) and snRNA systems disclosed herein can comprise a stem loop (SL) which includes compensatory modifications to a native snRNA stem loop (sometimes referred to herein as an “engineered stem loop” or “eSL”). These modifications result in increased stability of the engineered small nuclear ribonuclear protein complex (esnRNP) compared to snRNP comprising an unmodified stem loop. A SL disclosed herein can be derived from any snRNP such as U1-U12. In one embodiment, the SL is a human or mouse U7 SL. In one embodiment, the SL is a human SL. In one embodiment, the SL is a mouse SL. In some embodiments, the SL is a human and mouse SL. In some embodiments, the SL is a non-human SL, e.g., a mouse SL, a pig, a sheep SL, a goat SL, a cow SL, a dog SL, a cat SL, a horse SL, or a combination thereof. In some embodiments, the SL sequence is not a native stem loop sequence. In some embodiments, the nucleic acid sequence of the SL is at least 65%, 70%, 75%, 80%, 85%, 90%, 95%, 96%, 97%, 98%, 99% or 100% (or any percentage in between) is not a native stem loop sequence. Engineered stem loops are described in WO2023168458, the contents of which are incorporated herein by reference in its entirety for examples of SL sequences that may be used in the constructs described herein.

In some embodiments, a human SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 51)
GGCTTTCTGGCTCCTTACCGGAAAGCC,
(SEQ ID NO: 52)
GGCTTTCTGGGAGGTTACCGGAAAGCC,
(SEQ ID NO: 53)
GGCTTTCTGGCCTCCTTACCGGAAAGCC,
(SEQ ID NO: 54)
GGCTTTCTGGGGAGGTTACCGGAAAGCC,
(SEQ ID NO: 55)
GGCTTTCTGGCTGGCTACCGGAAAGCC,
(SEQ ID NO: 56)
GGCTTTCTGGCTTCCCCGGAAAGCC,
(SEQ ID NO: 57)
GGCTTTCTGGCTTCTTCCCGGAAAGCC,
(SEQ ID NO: 58)
GGCTTTCTGGCAACTTACCGGAAAGCC,
(SEQ ID NO: 59)
GGCTTTCTGGTTCGGTACCGGAAAGCC,
(SEQ ID NO: 60)
GGCTTTCTGGAAGCCTTACCGGAAAGCC,
(SEQ ID NO: 61)
GGCTTTCTGGCTTCTTACCGGAAAGCC,
or
(SEQ ID NO: 62)
GGCTTTCTGGCCTCCGCCGGAAAGCCCCT.

In some embodiments, a human SL comprises, consists essentially of, or consists of the nucleic acid sequence selected from the group consisting of SEQ ID NOS: 51-62. In some embodiments, a human SL comprises, consists essentially of, or consists of the nucleic acid sequence selected from the group consisting of SEQ ID NOS: 51-62, or a sequence having 1, 2, 3 or 4 substitutions, insertions or deletions relative thereto.

In some embodiments, a human SL comprises the sequence set forth in SEQ ID NO: 51. In some embodiments, a human SL comprises the sequence set forth in SEQ ID NO: 52. In some embodiments, a human SL comprises the sequence set forth in SEQ ID NO: 53. In some embodiments, a human SL comprises the sequence set forth in SEQ ID NO: 54. In some embodiments, a human SL comprises the sequence set forth in SEQ ID NO: 55. In some embodiments, a human SL comprises the sequence set forth in SEQ ID NO: 56. In some embodiments, a human SL comprises the sequence set forth in SEQ ID NO: 57. In some embodiments, a human SL comprises the sequence set forth in SEQ ID NO: 58. In some embodiments, a human SL comprises the sequence set forth in SEQ ID NO: 59. In some embodiments, a human SL comprises the sequence set forth in SEQ ID NO: 60. In some embodiments, a human SL comprises the sequence set forth in SEQ ID NO: 61. In some embodiments, a human SL comprises the sequence set forth in SEQ ID NO: 62.

In some embodiments, a murine SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 63)
GGCTTTCTGGCTCCTTACCGGAAAGCCCCT,
(SEQ ID NO: 64)
GGTTTTCTGACCTCCGTCGGAAAACCCCT,
(SEQ ID NO: 65)
GGTTTTCTGACCTCCTTCGGTCGGAAAACCCCT,
(SEQ ID NO: 66)
GGTTTTCTGACCTCCGTCGGAAAACC,
(SEQ ID NO: 67)
GGTTTTCTGACACTCCGTCGGAAAACCCCT,
(SEQ ID NO: 68)
GGTTTTCTGATCTCCATCGGAAAACCCCT,
or
(SEQ ID NO: 69)
GGTTTTCCGACCTCCGTCGGAAAACCCCT.

In some embodiments, a murine SL comprises, consists essentially of, or consists of the nucleic acid sequence selected from the group consisting of SEQID NOS: 63-69. In some embodiments, a murine SL comprises, consists essentially of, or consists of the nucleic acid sequence selected from the group consisting of SEQID NOS: 63-69, or a sequence having 1, 2, 3 or 4 substitutions, insertions or deletions relative thereto.

In some embodiments, a murine SL comprises the sequence set forth in SEQ ID NO: 63. In some embodiments, a murine SL comprises the sequence set forth in SEQ ID NO: 64. In some embodiments, a murine SL comprises the sequence set forth in SEQ ID NO: 65. In some embodiments, a murine SL comprises the sequence set forth in SEQ ID NO: 66. In some embodiments, a murine SL comprises the sequence set forth in SEQ ID NO: 67. In some embodiments, a murine SL comprises the sequence set forth in SEQ ID NO: 68. In some embodiments, a murine SL comprises the sequence set forth in SEQ ID NO: 69.

In some embodiments, a human or murine SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 70)
GGCTTTCTGGCACTCCACCGGAAAGCCCCT,
(SEQ ID NO: 71)
GGCTTTCTGGCACTCCGCCGGAAAGCCCCT,
or
(SEQ ID NO: 72)
GGCTTTCTGGCCTCCACCGGAAAGCCCCT.

In some embodiments, a human or murine SL comprises, consists essentially of, or consists of the nucleic acid sequence selected from the group consisting of SEQ ID NOS: 70-72. In some embodiments, a human or murine SL comprises, consists essentially of, or consists of the nucleic acid sequence selected from the group consisting of SEQ ID NOS: 70-72, or a sequence having 1, 2, 3 or 4 substitutions, insertions or deletions relative thereto.

In some embodiments, a human or murine SL comprises the sequence set forth in SEQ IID NO: 70. In some embodiments, a human or murine SL comprises the sequence set forth in SEQ IID NO: 71. In some embodiments, a human or murine SL comprises the sequence set forth in SEQ IID NO: 72.

In some embodiments, a dog or cat SL comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleotide sequence GGTTTTCCGGTCTCCACCGGAAAGCCCCC (SEQ ID NO: 73). In some embodiments, a dog or cat SL comprises, consists essentially of, or consists of the nucleic acid sequence of SEQ ID NO: 73. In some embodiments, a dog or cat SL comprises, consists essentially of, or consists of the nucleic acid sequence of SEQ ID NO: 73, or a sequence having 1, 2, 3 or 4 substitutions, insertions or deletions relative thereto.

In some embodiments, a cow, sheep, or goat SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 74)
GGCTTTCCGGTCTCCACCGGAAAGCCCCT,
or
(SEQ ID NO: 75)
GGCTTTCCGGCCTCCGCCGGAAAGCCCCT.

In some embodiments, a cow, sheep, or goat SL comprises, consists essentially of, or consists of the nucleic acid sequence selected from the group consisting of SEQ ID NOS: 74 and 75. In some embodiments, a cow, sheep, or goat SL comprises, consists essentially of, or consists of the nucleic acid sequence selected from the group consisting of SEQ ID NOS: 74 and 75, or a sequence having 1, 2, 3 or 4 substitutions, insertions or deletions relative thereto.

In some embodiments, a cow, sheep, or goat SL comprises the sequence set forth in SEQ ID NO: 74. In some embodiments, a cow, sheep, or goat SL comprises the sequence set forth in SEQ ID NO: 75.

In some embodiments, a pig SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 76)
GGTTTTCCGGTCTCCACCGGAAAACCCTT,
(SEQ ID NO: 77)
GGTTTTCCGTGCTCCCACGGAAAACCCTT,
(SEQ ID NO: 78)
GGTTTTCCGGCCTCCGCCGGAAAACCCTT,
(SEQ ID NO: 79)
GGTTTTCCGTGACTCCCACGGAAAACCCTT,
or
(SEQ ID NO: 80)
GGTTTTCCGGCACTCCGCCGGAAAACCCTT.

In some embodiments, a pig SL comprises, consists essentially of, or consists of the nucleic acid sequence selected from the group consisting of SEQ ID NOS: 76-80. In some embodiments, a pig SL comprises, consists essentially of, or consists of the nucleic acid sequence selected from the group consisting of SEQ ID NOS: 76-80, or a sequence having 1, 2, 3 or 4 substitutions, insertions or deletions relative thereto.

In some embodiments, a pig eL comprises the sequence set forth in SEQ ID NO: 76. In some embodiments, a pig SL comprises the sequence set forth in SEQ ID NO: 77. In some embodiments, a pig SL comprises the sequence set forth in SEQ ID NO: 78. In some embodiments, a pig SL comprises the sequence set forth in SEQ ID NO: 79. In some embodiments, a pig SL comprises the sequence set forth in SEQ ID NO: 80.

In some embodiments, a horse SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 81)
GGTCTTCCGGTCTCCTCCGGAAGGCCCCC,
or
(SEQ ID NO: 82)
GGTCTTCCGGCTCCCCGGAAGGCCCCC.

In some embodiments, a horse SL comprises, consists essentially of, or consists of the nucleic acid sequence selected from the group consisting of SEQ ID NO: 81 and 82. In some embodiments, a horse SL comprises, consists essentially of, or consists of a nucleic acid sequence selected from the group consisting of SEQ ID NO: 81 and 82, or a sequence having 1, 2, 3 or 4 substitutions, insertions or deletions relative thereto.

In some embodiments, a horse SL comprises the sequence set forth in SEQ ID NO: 81. In some embodiments, a horse SL comprises the sequence set forth in SEQ ID NO: 82.

In some embodiments, a sheep SL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 83)
GGCTTTCCGTGCTCCCACGGAAAGCCCCT,
(SEQ ID NO: 84)
GGCTTTCCGTGACTCCCACGGAAAGCCCCT,
or
(SEQ ID NO: 85)
GGCTTTCCGGCACTCCGCCGGAAAGCCCCT.

In some embodiments, a sheep SL comprises, consists essentially of, or consists of the nucleic acid sequence selected from the group consisting of SEQ ID NOS: 83-85. In some embodiments, a sheep SL comprises, consists essentially of, or consists of a nucleic acid sequence selected from the group consisting of SEQ ID NOS: 83-85, or a sequence having 1, 2, 3 or 4 substitutions, insertions or deletions relative thereto.

In some embodiments, a sheep SL comprises the sequence set forth in SEQ ID NO: 84. In some embodiments, a sheep SL comprises the sequence set forth in SEQ ID NO: 85. In some embodiments, a sheep SL comprises the sequence set forth in SEQ ID NO: 86.

In some embodiments, engineered stem loops provide for enhanced stability of an snRNA relative to an snRNA comprising a native stem loop. In some embodiments is a native snRNA stem loop comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 86)
Ggttttctgacttcggtcggaaaacccct,
(SEQ ID NO: 87)
ggttttctgacttcggtcggaaaacc,
(SEQ ID NO: 88)
Ggctttctggctttttaccggaaagcc,
(SEQ ID NO: 89)
ggctttctggctttttaccggaaagccCCT,
(SEQ ID NO: 90)
GGCTTTCCGGCCTCCGCCGGAAAGCCCCT,
or
(SEQ ID NO: 91)
GGCTTTCCGGCCTCCGCCGGAAAGCC.

In some embodiments, the stem loop is a native snRNA stem loop comprises, consists essentially of, or consists of the nucleic acid sequence selected from the group consisting of SEQ ID NOS: 86-91. In some embodiments, the stem loop is a native snRNA stem loop comprises, consists essentially of, or consists of the nucleic acid sequence selected from the group consisting of SEQ ID NOS: 86-91, or a sequence having 1, 2, 3 or 4 substitutions, insertions or deletions relative thereto.

In some embodiments, the stem loop is a native snRNA stem loop comprises the sequence set forth in SEQ ID NO: 86. In some embodiments, the stem loop is a native snRNA stem loop comprises the sequence set forth in SEQ ID NO: 87. In some embodiments, the stem loop is a native snRNA stem loop comprises the sequence set forth in SEQ ID NO: 88. In some embodiments, the stem loop is a native snRNA stem loop comprises the sequence set forth in SEQ ID NO: 89. In some embodiments, the stem loop is a native snRNA stem loop comprises the sequence set forth in SEQ ID NO: 90. In some embodiments, the stem loop is a native snRNA stem loop comprises the sequence set forth in SEQ ID NO: 91.

### 5′ Interaction Stability Domain

The SL disclosed herein can possesses more effective folding and annealing properties with a 5′ interaction stability domain (5′ISD) and this in turn results in increased stability of the esnRNA compared to a non-engineered snRNA. The 5′ ISD has nucleotides that are complementary to the nucleotides within the SL, and, without wishing to be bound by theory, an interaction between the 5′ISD and SL is predicted to form secondary structure that protects the 5′ end of an snRNA. In some aspects the 5′ ISD anneals and/or hybridizes to an SL of the disclosure. In some aspects the 5′ISD is a sequence having complementarity and/or reverse complementarity to a sequence present in an SL of the disclosure. In some aspects a 5′ISD disclosed herein can comprise or consist of one of the following nucleotide sequences:

GGAGT,
CCTCT,
GGAGGT,
CCTCCT,
AGCCAG,
GGAAG,
GAAGAAG,
GTTG,
CCGAA,
TAAGGAG,
GAAG,
or
GGCTT.

### Sm Binding Domains

The snRNA systems disclosed herein utilize an Sm binding domain (SmBD). The Sm protein ring that assembles around the Sm binding domain (SmBD) to form an snRNP includes SmB/B′, SmD1, SmD2, SmD3, SmE, SmF, and SmG. The U7 Sm binding site recruits endogenous RNA binding factors and can be replaced with a non-U7 snRNA to make the esnRNA more stable. In one embodiment, the SmBD a U1 SmBD, a U2 SmBD, a U4 SmBD, or a SmBD. In another embodiment, the SmBD is derived from a pseudo snRNA. In another embodiment, the SmBD is a nucleotide sequence comprising ATTTTT. In another embodiment, the SmBD comprises the nucleotide sequence AATTTTTGG, AATTTGTGG, AATTTGTGG, AATTTCTGG, GATTTTTGG, AATTTTTGA, AATTTTTTG, AATTTTTGGAGCA (SEQ ID NO: 127), or AATTTTTGGAGTA (SEQ ID NO: 128). In some embodiments, the SmBD comprises the nucleotide sequence AATTTTTGG, AATTTGTGG, AATTTGTGG, AATTTCTGG, GATTTTTGG, AATTTTTGA, AATTTTTTG, AATTTTTGGAGCA (SEQ ID NO: 127), or AATTTTTGGAGTA (SEQ ID NO: 128), or a sequence having 1, 2 or 3 insertions, deletions or substitutions relative thereto.

### Promoter Sequences

Gene therapy and RNA-targeting snRNA gene therapy compositions of the disclosure comprise promoter sequences derived from an snRNA. A “promoter” is a regulatory sequence that is a region of a polynucleotide sequence at which initiation and rate of transcription are controlled. It may contain genetic elements at which regulatory proteins and molecules may bind such as RNA polymerase and other transcription factors.

The snRNA systems disclosed herein may comprise an snRNA promoter from any of U1-U12. In one embodiment, the snRNA promoter is a U7 promoter. In another embodiment, the U7 promoter is a human U7 promoter (hU7) or a mouse U7 promoter (mU7). In another embodiment, the U7 promoter is an endogenous human U7 promoter at least 65%, 70%, 75%, 80%, 85%, 90%, 95%, 96%, 97%, 98%, 99% or 100% (or any percentage in between) identical to SEQ ID NO: 102: TACTGCCGAATCCAGGTCTCCGGGCTTAACAACAACGAAGGGGCTGTGACTGGCTG CTTTCTCAACCAATCAGCACCGAACTCATTTGCATGGGCTGAGAACAAATGTTCGCG AACTCTAGAAATGAATGACTTAAGTAAGTTCCTTAGAATATTATTTTTCCTACTGAA AGTTACCACATGCGTCGTTGTTTATACAGTAATAGGAACAAGAAAAAAGTCACCTA AGCTCACCCTCATCAATTGTGGAGTTCCTTTATATCCCATCTTCTCTCCAAACACATA CGCA. In some embodiments, the promoter comprises the sequence of SEQ ID NO: 102, or a sequence having 1, 2, 3, 4, 5, 6, 7, 8, 9 or 10 insertions, substitutions or deletions relative thereto.

In one embodiment, the snRNA promoter is a U1 promoter. In another embodiment, the U1 promoter is a human U1 promoter or a mouse U1 promoter.

In another embodiment, for example those embodiments in which a polynucleotide comprises multiple snRNAs, the same snRNA promoter drives expression of each snRNA insert.

In another embodiment, each snRNA insert is the same. In another embodiments, one or more snRNA inserts are different. In another embodiment, different snRNA promoters drive each snRNA insert. In one embodiment, a 2×snRNA comprises a mouse U7 promoter driving one copy of an snRNA insert and a mouse U1 promoter drives the other copy of an snRNA insert.

In other aspects, the snRNA promoter is a PolII promoter or a PolIII promoter.

In other aspects, the snRNA promoter comprises, consists essentially of, or consists of a nucleic acid sequence at least 65%, 70%, 75%, 80%, 85%, 90%, 95%, 96%, 97%, 98%, 99% or 100% (or any percentage in between) identical to a promoter and/or promoter sequence listed in Table, which follows:

TABLE 5
Illustrative Promoter Sequences

| | | SEQ |
|---|---|---|
| Name | Sequence | ID NO |
| hU1 | AAGGACCAGCTTCTTTGGGAGAGAACAGACGCAGGGGGGGGAGG | 92 |
| | GAAAAAGGGAGAGGCAGACGTCACTTCCCCTTGGCGGCTCTGGCA | |
| | GCAGATTGGTCGGTTGAGTGGCAGAAAGGCAGACGGGGACTGGG | |
| | CAAGGCACTGTCGGTGACATCACGGACAGGGCGACTTCTATGTAG | |
| | ATGAGGCAGCGCAGAGGCTGACGTCTTCGCCACTTGCTGCTTCAC | |
| | CACGAAGGAGTTCCCGTGCCCTGGGAGCGGGTTCAGGACCGCTGA | |
| | TCGGAAGTGAGAATCCCAGCTGTGTGTCAGGGCTGGAAAGGGCTC | |
| | GGGAGTGCGCGGGGCAAGTGACCGTGTGTGTAAAGAGTGAGGCG | |
| | TATGAGGCTGTGTCGGGGCAGAGGCacaacgtttc | |
| hU2 | CCACGCCCTCTGTGAAAGGGCGGGGCATGCAAATTCGAAATGAAA | 93 |
| | GCCCGGGAACGCCGGAAGAAGCACGGGTGTAAGATTTCCCTTTTC | |
| | AAAGGCAGAGAATAAGAAATCAGCCCGAGAGTGTAAGGGCGTCA | |
| | ATAGCGCTGTGGACGAGACAGAGGGAATGGGGCAAGGAGCGAGG | |
| | CTGGGGCTCTCACCGCGACTTGAATGTGGATGAGAGTGGGACGGT | |
| | GACGGCGGGCGCGAAGGCGAGCGC | |
| hU4 | TTCGCAGTCTCTGAATTAAGTCTATTAGCATGTTCCTCCCATAGTG | 94 |
| | CTTTGCTTCATATCAACAAAAACCTAGCTAAGTGAAATCAGCAAC | |
| | GATATGCAGAAACCACCTACGCAGGTCACAAACATCTTTCTATGA | |
| | TTGTATAATTTTCAAGCAAGCAATAAGTGAAGATTTTTCCATAGGC | |
| | CCTAAACTCACCTTTGCGAAATAGGAAGCTGGTTTATTGGGAGTG | |
| | ATGAGCAGGGGGCGTAACAAATT | |
| hU5 | GCAGCAAGGCCTCCACTTCACCCCCTAAAGGTTGCCCCAAGAGCA | 95 |
| | CCGTGTGACTGCTAAGGTATTTCCGGAGTCTAAAGACGATTATTCA | |
| | GGTCTCATTTGCATACCCATAATACACTGCAAACAGTATTTTTTTC | |
| | GGAAAAACATTTATATATTGCTTGACATTTTTAAGTATGAGAATTT | |
| | TGCATGCAGAATTTTTTTGTATAAACTTTCTCAGGTAGTAACCCTT | |
| | GGGATTAGTAGACACCATCAGTGTACTAGGAATTGCAGTTACCCG | |
| | AAAATTGAGTTACAGAAGTAACTGGT | |
| hU6 | Gtttattacagggacagcagagatccagtttggttaattaaggtaccgagggcctatttcccatgattccttcatat | 96 |
| | ttgcatatacgatacaaggctgttagagagataattagaattaatttgactgtaaacacaaagatattagtacaaaa | |
| | tacgtgacgtagaaagtaataatttcttgggtagtttgcagttttaaaattatgttttaaaatggactatcatatgc | |
| | ttaccgtaacttgaaagtatttcgatttcttggctttatatatcttgtggaaaggacgaaacacc | |
| h7sk | CTGCAGTATTTAGCATGCCCCACCCATCTGCAAGGCATTCTGGATA | 97 |
| | GTGTCAAAACAGCCGGAAATCAAGTCCGTTTATCTCAAACTTTAG | |
| | CATTTTGGGAATAAATGATATTTGCTATGCTGGTTAAATTAGATTT | |
| | TAGTTAAATTTCCTGCTGAAGCTCTAGTACGATAAGTAACTTGACC | |
| | TAAGTGTAAAGTTGAGATTTCCTTCAGGTTTATATAGCTTGTGCGC | |
| | CGCCTGGGTACCTC | |
| tRNA | CAGGACTAGTCTTTTAGGTCAAAAAGAAGAAGCTTTGTAACCGTT | 98 |
| (Val) | GGTTTCCGTAGTGTAGTGGTTATCACGTTCGCCTAACACGCGAAAG | |
| | GTCCCCGGTTCGAAG | |
| mU1 | TTGTTCCTCTTAGTGTTAATTCACACTAAAGACTGTGCATCCGACT | 99 |
| | CCTACATTTATGAAAGTAAATGCCTGTTGTTAGAACAAAAAAGGC | |
| | TACAGAACAAAAAACAAAGCGAAATACCATCTGCTTTAGGTTCAG | |
| | TGTGGTATTTTCCCGCTGACAGGGAGGCGGGTTTTTGGGTACAGG | |
| | AAACGAGTCACTATGGAGGCGGTACTATGTAGATGAGAATTCAGG | |
| | TGCAAACTGGGAAAAGCAACTGCTTCCAAATATTTGTGATTTTTAC | |
| | AGTGTAGTTTTGGAAAAACTCTTAGCCTACCAATTCTTCTAAGTGT | |
| | TTTAAAATGTGGGAGCCAGTACACATGAAGTTATAGAGTGTTTTA | |
| | ATGAGGCTTAAATATTTACCGTAACTATGAAATGCTACGCATATCA | |
| | TGCTGTTCAGGCTCCGTGGCCACGCAACTC | |
| mU7+ | TaacaacataggagctgtgattggctgttttcagccaatcagcactgActcatttgcatagcctttacaagcggt | 100 |
| extra | cacaaactcaagaaacgagcggttttaatagtcttttagaatattgtttatcgaaccgaataaggaactgtgctttg | |
| 3′ | tgattcacatatcagtggaggggtgtggaaatggcaccttgatctcaccctcatcgaaagtggagttgatgtcct | |
| | Tccctggctcgctacagacgcacttccgcaa | |
| mU7 | taacaacataggagctgtgattggctgttttcagccaatcagcactgActcatttgcatagcctttacaagcggtc | 101 |
| | acaaactcaagaaacgagcggttttaatagtcttttagaatattgtttatcgaaccgaataaggaactgtgctttgt | |
| | gattcacatatcagtggaggggtgtggaaatggcaccttgatctcaccctcatcgaaagtggagttgatgtcct | |
| | Tccctggctcgctacagacgcacttccgc | |
| hU7 | TACTGCCGAATCCAGGTCTCCGGGCTTAACAACAACGAAGGGGCT | 102 |
| | GTGACTGGCTGCTTTCTCAACCAATCAGCACCGAACTCATTTGCAT | |
| | GGGCTGAGAACAAATGTTCGCGAACTCTAGAAATGAATGACTTAA | |
| | GTAAGTTCCTTAGAATATTATTTTTCCTACTGAAAGTTACCACATG | |
| | CGTCGTTGTTTATACAGTAATAGGAACAAGAAAAAAGTCACCTAA | |
| | GCTCACCCTCATCAATTGTGGAGTTCCTTTATATCCCATCTTCTCTC | |
| | CAAACACATACGCA | |
| mU5 | TGCCAACAGCTTTGAAATCCTCTGTGCTCTTGTGCCAATCCCACCC | 103 |
| | TCAGAACAGGGAGTCTGTCGGTTATGTTTTCGTCCTTTACAGTTCT | |
| | TCGCCGTCTAATCCATTGGAAACCTCCTTTATTTAAAGGAAACCGT | |
| | GTCCAAATTCAGTGTACAGAGGTGCAAACCTCCGCCCCGCTCCTTG | |
| | TAGGAAGCCCACGGGAACTGGGCCAATCGGACGGCGCTTACTCGG | |
| | CTCATTTACATACCCATAACACACCGCGGCTAATGCAAATATTTTC | |
| | GTGAAAAGATCTTCCATTACTCGAAGTTTTAAAACTAAAAACAAA | |
| | ATATTATTTCAAAAGATCATGAAGAAATTTCTGTCGGTAACAGCA | |
| | GTTTCAATTGATAAATCACCATCACTTATCTAGGAGGTTCTGTTAC | |
| | TCTAGAAGTGAATTAAGCAGGACAGCTGT | |
| mU2 | Gggcggggcatgcaaataactgctctgtggaactctgggagcaaaaacaaaaaactgcaaccaaaacttctc | 104 |
| | ggcctccttgaatcttacaggcttttcgtggcgtaaaggtggtgtactcaatgaagaggagagtctgtgttggct | |
| | gcatgtttgagtcggttggttggtgactgtgaatTAAAGGTGTGGtcggtgttgagtgtatggggcgt | |
| | gtgggcgtagttcggt | |
| mU6 | cgacgccgccatctctaggcccgcgccggccccctcgcacagacttgtgggagaagctcggctactcccctg | 105 |
| | ccccggttaatttgcatataatatttcctagtaactatagaggcttaatgtgcgataaaagacagataatctgttct | |
| | ttttaatactagctacattttacatgataggcttggatttctataagagatacaaatactaaattattattttaaaa | |
| | aacagcacaaaaggaaactcaccctaactgtaaagtaattgtgtgttttgagactataaatatcccttggagaaaag | |
| | ccttgttt | |
| mH1 | CATgCAAATTACGCGCTgTGCTTTGTGGGAAATCACCCTAAACGTA | 106 |
| | AAATTTATTCCTCTTTcGAGCCTTATAGTGGcGGCCGGTCTACACCC | |
| | TAAA | |
| rU1 | TTACTTCATACTAAAGGCTGTGCATCCGACTCCTAAGTTGATGAAG | 135 |
| | GAAAATGCCTAGTGTTCTTGGAGGCTACAAAACAAAAGACAAAGC | |
| | TAACTACCATCTGCTTATGGGTTCATTGGTATTTTCCAGCTGGCAG | |
| | GGAGGCGGGTTTCCGAGTACAGGAAATGAGTCTCTATGGAGGCGG | |
| | TGCTATGTAGATGAGAATTCAGGAGCAAACTGGGAAAAGCAGCTG | |
| | CTTCCAAATATTTGTGATTTTTGGAGTGTAGTTTTGGGGAAACTCG | |
| | CAGCCTACCAATTCTCCTAAGTGCTTTAGAATATGGAGAGACACT | |
| | GTACATAAAGATATAGAGCTTTTTAATGGAGGCTTAAATTTATACC | |
| | GTATCTACAAAATGCTACATTCACAATGCAGTTCAGGCTCTGTGGC | |
| | ATTGCAACTC | |
| bU7 | GCGCAGGAGCCGCCGAGCTCTTGCTGCGAAGCCTTGTCTGCGTTCT | 136 |
| | TAAAAACTAAAGGGGGCGTGACTGGCTTCCTTATCAGCCAATCGG | |
| | CATCGGGTCATTTGCATAGGGCCCCATCCACCGTTCGTAAACTCTA | |
| | GCCGCGAAGGATTTAAGAGAGTTCCTTAGAACACGGTCTTCCCCT | |
| | CCGAAAGTCGCAGTCTGCCTCGTTATCTGTAGAGCAATAGGAGCT | |
| | AGGAACCGGCCGTCGGGGCTCACCCTCACTGACAGCGGGGTTGGT | |
| | GGCTATGCCGTGCGATCTCCGGGTGCTACGTCTG | |
| SU1 | CGCCGTGCCCTGCTCTGCCCTCCGCACGCTGCTCAGACTCCACACC | 137 |
| | CGTAACGAAGCTCCACCGAATTTTTCGTCTGTCTGTGAAGACGAA | |
| | GAATTGAAAAATCTCAGCTACACTTGGTCTTTGGGGTCGGCGCCA | |
| | GGCAGAGCTAGAACGAACTAGACGCTTGTCCGAAAGCAGTGACGT | |
| | CAAATGACAAGGGACAAGAGGTGGGGATATGTAGATGAGGGGAG | |
| | CGGTGGCTGCTGGCTGGATTTCGTCTTTCGCCGGGTAGAATTTCCC | |
| | CTGAACGTCCAAAGGATGTATCTTGGTCACTGTGAAATAAATAAT | |
| | CCTTCAGGTTCCCATCGAAGGCCGAAATAGCTTGGAATATAATTG | |
| | ACCATAACTCTGTTGTGTGCTGAGTTCGAGGGTGACGTCCCAGACT | |
| | AAGCTTACAACTC | |
| eqU1 | TGCCGCGCAGAATGCAGACTCATCCTGACAAGTCCACGCCAGAAC | 138 |
| | CTTTGCGTCTGCCCGGAAAGGCAAGGAAGTTTAAAGCCTTTCCAG | |
| | ATACCCCTATGGATGATTGTCTTTTAGATCGTCCTGAGGGCTAGCT | |
| | AGAGCGAATTCACCGCTTCTCCGAGAAGCAGAATGACGTCGAGTG | |
| | ACGACAGTGATGACAAGAGGCGGGGATATGTAGATGAAAGACAC | |
| | GGTGACTGCCGGTTGGAGTGAGTTCTTCATGGGGAACACTTTCCAC | |
| | TGAATATCCAAAAGGATGTACCCTGATCTCTGTCATATAAATAACC | |
| | CTTCAATTTCGAACTTAAGGCCTAAAGAACTCGGAATATAATTGA | |
| | CCATGACTTTATAGTGTGTTGAAGACGATGGTGAGGTGCCAGGCG | |
| | AGGGGAGCAACTC | |

In some embodiments, the snRNA promoter comprises the sequence set forth in SEQ ID NO: 92. In some embodiments, the snRNA promoter comprises the sequence set forth in SEQ ID NO: 93. In some embodiments, the snRNA promoter comprises the sequence set forth in SEQ ID NO: 94. In some embodiments, the snRNA promoter comprises the sequence set forth in SEQ ID NO: 95. In some embodiments, the snRNA promoter comprises the sequence set forth in SEQ ID NO: 96. In some embodiments, the snRNA promoter comprises the sequence set forth in SEQ ID NO: 97. In some embodiments, the snRNA promoter comprises the sequence set forth in SEQ ID NO: 98. In some embodiments, the snRNA promoter comprises the sequence set forth in SEQ ID NO: 99. In some embodiments, the snRNA promoter comprises the sequence set forth in SEQ ID NO: 100. In some embodiments, the snRNA promoter comprises the sequence set forth in SEQ ID NO: 101. In some embodiments, the snRNA promoter comprises the sequence set forth in SEQ ID NO: 102. In some embodiments, the snRNA promoter comprises the sequence set forth in SEQ ID NO: 103. In some embodiments, the snRNA promoter comprises the sequence set forth in SEQ ID NO: 104. In some embodiments, the snRNA promoter comprises the sequence set forth in SEQ ID NO: 105. In some embodiments, the snRNA promoter comprises the sequence set forth in SEQ ID NO: 106. In some embodiments, the snRNA promoter comprises the sequence set forth in SEQ ID NO: 135. In some embodiments, the snRNA promoter comprises the sequence set forth in SEQ ID NO: 136. In some embodiments, the snRNA promoter comprises the sequence set forth in SEQ ID NO: 137. In some embodiments, the snRNA promoter comprises the sequence set forth in SEQ ID NO: 138. In some embodiments, the snRNA promoter comprises the sequence set forth in in any one of SEQ ID NOS: 92-106 or 135-138, or a sequence having 1, 2, 3, 4, 5, 6, 7, 8, 9 or 10 insertions, substitutions or deletions relative thereto.

### Terminator Sequences

The snRNA systems (i.e., snRNAs, as well as vectors, polynucleotides and complexes comprising or encoding same) disclosed herein may comprise an snRNA downstream terminator (DT). Downstream terminators can define the end of a transcriptional unit, such as an esnRNA or snRNA. In another embodiment the snRNA DT is a U7 DT comprising the sequence CCTCTTATGATGTTTGTTGCCAATGATAGATTGTTTTCACTGTGCAAAAATTATGGGT AGTTTTGGTGGTCTTGATGCAGTTGTAAGCTTGGAG (SEQ ID NO: 114). In another embodiment the snRNA DT is a U7 DT comprising the sequence of SEQ ID NO: 114, or a sequence having 1, 2, 3, 4 or 5 insertions, substitutions or deletions relative thereto.

In one embodiment, the snRNA comprises the SL or eSL, one or more promoters, the TS targeting a SOD1 RNA molecule, the SmBD, the 5′ISD, and the DT. In another embodiment, the snRNA comprises a native stem loop, one or more promoters, the TS targeting SOD1, the SmBD, and the DT. The promoter and DT sequences provided herein may be mixed and matched in any combination.

In some embodiments, the DT comprises, consists essentially of, or consists of a nucleic acid sequence at least 65%, 70%, 75%, 80%, 85%, 90%, 95%, 96%, 97%, 98%, 99% or 100% (or any percentage in between) identical to a DT sequence listed in Table 6, which follows:

TABLE 6
Illustrative DT Sequences

| | | SEQ |
|---|---|---|
| Name | Sequence | ID NO |
| hU1 | ACTTTCTGGAGTTTCAAAAACAGACTGTACGCCAAGGGT | 107 |
| | CATATCTTTTT | |
| hU2 | CCGGGGATACAACGTGTTTCCTAAAAGTAGAGGGAGGTA | 108 |
| | AGAGACGGTAG | |
| hU4 | CTGAATTTTCTTGCAGTTGAACAACAGAGGCTTTTTTTGT | 109 |
| | GTGTGTGGGG | |
| hU5 | ATATGTGGTAATCCAACAATAGAAATTATTTTTAAGTTTG | 110 |
| | TGTGTTCCTT | |
| hU6 | TTTTTT | N/A |
| h7sk | TTTTTT | N/A |
| tRNA(Val) | TTTTTT | N/A |
| mU1 | GTTTACTTGGTTTTAAAAATAGCTTGCACTAGCGATACGG | 111 |
| | AATATGGTTATTAGGTTTGTTAGGCATCATGTCGTGTCTT | |
| | ACTATAGAAAAATAACGTAGTGTTCATTTTAGCCTGCCTG | |
| | TATGTGTTAATTTGTCCTTATTGCGCATTGTTCTTGTTAAG | |
| | TCTTCTGTAAGGAGTTGCGGGTTTCAAACTGTCAGTCTGA | |
| | GAGCA | |
| mU7+ | Cccaatttcactggtctacaatgaaagcaaaacagttctcttccccgctccccggtgtgtgagag | 112 |
| | gggctttgatccttctctggtttcctaggaaacgcgtatgtgtac | |
| mU7 | Cccaatttcactggtctacaatgaaagcaaaacagttctcttccccgctccccggtgtgtgagag | 113 |
| | gggctttgatccttctctggtttcctaggaaacgcgtatgtg | |
| hU7 | CCTCTTATGATGTTTGTTGCCAATGATAGATTGTTTTCACT | 114 |
| | GTGCAAAAATTATGGGTAGTTTTGGTGGTCTTGATGCAGT | |
| | TGTAAGCTTGGAG | |
| hU7 | CTTATGATGTTTGTTGCCAATGATAGATTGTTTTCACTGTG | 115 |
| | CAAAAATTATGGGTAGTTTTGGTGGTCTTGATGCAGTTGT | |
| | AAGCTTGGAG | |
| mU5 | CCACATTTTGTGTTTAAAAATAGAAATATTTAAGTGAGAT | 116 |
| | CAGTTTAAATCTGCTTTATCTAGGGTGTCTAACTGCTTGC | |
| | ATCTTTTTAACTTTTCCTTATTTGTGAGAAGGTCTGTAAAA | |
| | CTTATTATATGTTAGTACACTGTAGCTGTCTTCAGACACT | |
| | CCAGAAGAGGGATTCAGATCACCTTACAGATGGTTTTGA | |
| mU2 | CCCTCTGGGGAgtaaagttggttttaaagtcagagcatggtgattgtagggcagtcca | 117 |
| | acttttttaaatatgctgtg | |
| mU6 | TTTTTT | N/A |
| mH1 | TTTTTT | N/A |
| rU1 | ATTTGTTTGGTACTAAAGATAGTTATCAGCCGAACCAGAA | 139 |
| | GGCTAAAATGGCTTCTGATACTTACTTGGCCAATGCCTTT | |
| | TCCCTTTATACTGCTATTGCTTTGTATTCTGAAAAGCATCT | |
| | TAGTGGTTTTTAACTTTCTCTACGTTCCCTCTGCACGTTGA | |
| | GTCTTGAGTTATGTTAAATGGTACGGTACCTAGGATTG | |
| bU7 | CCCACACAAGTGTTTATGAACAATAGAATAATATGTGGG | 140 |
| | TGGGAAGCTTGGTTTTTTTTGATACGGGTTTTTTTTTTTTT | |
| | TTTGGTCTACGTGTTTAGTCCGTTCTTGACGGAGAAGACC | |
| | CGGGAGGTTGGGATTCTTGTCTGGAAATTTCCGGCTCTGT | |
| | CTGATGTAGTTTCCCGTTCGCCTCCCTTCTTTGCTGCGAG | |
| SU1 | GCGTGGTTTGTGCAAGGAAAGACATAGATGCTAAGTTTG | 141 |
| | GAGCTCTGCTAATTTCCTGTTTTTGTAGTCGTTTTCGGGTT | |
| | TATGAGGCAGAGGCAGTAAGCAGTTTTCCTGCAACATGG | |
| | ATAAGCTAGTTTGGACTTTTGGTAAAGGATTTTTTTGTAA | |
| | TGTGGGAGACTTGCTTTTGATCCCTGGTTTGGGAAATTCT | |
| | C | |
| eqU1 | ACAAACTTGGTCTTAAAAGAAGACTACTTGTTTATGTTCT | 142 |
| | TCTTTCTCTAACTTTTATTTGGGGTTTAGTGGCTTTCTAAC | |
| | ATGGTCATTATGAGGTAAGACCTTGGGGTTGGCCTATTAA | |
| | TTAACACCTTGGGGCTGGCCGTATGGCTGAGTGGTTAAGT | |
| | TTGTGTGCTCTGCTTCGGGTTTGGCCTGTTCTAATTCTG | |

In some embodiments, the DT comprises the sequence set forth in SEQ ID NO: 107. In some embodiments, the DT comprises the sequence set forth in SEQ ID NO: 108. In some embodiments, the DT comprises the sequence set forth in SEQ ID NO: 109. In some embodiments, the DT comprises the sequence set forth in SEQ ID NO: 110. In some embodiments, the DT comprises the sequence set forth in SEQ ID NO: 111. In some embodiments, the DT comprises the sequence set forth in SEQ ID NO: 112. In some embodiments, the DT comprises the sequence set forth in SEQ ID NO: 113. In some embodiments, the DT comprises the sequence set forth in SEQ ID NO: 114. In some embodiments, the DT comprises the sequence set forth in SEQ ID NO: 115. In some embodiments, the DT comprises the sequence set forth in SEQ ID NO: 116. In some embodiments, the DT comprises the sequence set forth in SEQ ID NO: 117. In some embodiments, the DT comprises the sequence set forth in SEQ ID NO: TTTTTT. In some embodiments, the DT comprises the sequence set forth in SEQ ID NO: 139. In some embodiments, the DT comprises the sequence set forth in SEQ ID NO: 140. In some embodiments, the DT comprises the sequence set forth in SEQ ID NO: 141. In some embodiments, the DT comprises the sequence set forth in SEQ ID NO: 142. In some embodiments, the DT comprises, consists essentially of, or consists of the nucleic acid sequence set forth in any one of SEQ ID NOS: 107-117 or 139-142, or a sequence having 1, 2, 3, 4 or 5 insertions, substitutions or deletions relative thereto.

In one embodiment, the snRNA is delivered by an AAV vector.

In some embodiments, the AAV vector comprises multiple copies of the sequence encoding the snRNA. In some embodiments, the multiple copies of the sequence encoding the snRNA are 2, 3, 4, 5, 6, 7, 8, 9, or 10 copies (2×, 3×, 4×, 5×, 6×, 7×, 8×, 9× or 10×) of the snRNA. In some embodiments, the multiple copies of the sequence encoding the snRNA are 4 or more copies of the snRNA. In some embodiments, the AAV comprises one copy of the sequence encoding the snRNA. In some embodiments, the AAV comprises two copies of the sequence encoding the snRNA. In some embodiments, the AAV comprises three copies of the sequence encoding the snRNA. In some embodiments, the AAV comprises four copies of the sequence encoding the snRNA. In some embodiments, the AAV comprises five copies of the sequence encoding the snRNA.

In some embodiments, the AAV comprises sequences encoding multiple snRNA, and the two or more of the snRNA are not the same. For example, snRNA that do not have the same targeting sequence are not the same. As a further example, snRNA that comprise different features other than the targeting sequence, e.g. different SL, are also not the same. In some embodiments, the AAV comprises sequences encoding two snRNA, and the two snRNA are not the same. In some embodiments, the AAV comprises sequences encoding three snRNA, and the three snRNA are not the same. In some embodiments, the AAV comprises sequences encoding four snRNA, and the four snRNA are not the same. In some embodiments, the AAV comprises sequences encoding five snRNA, and the five snRNA are not the same.

In some embodiments, each sequence encoding the snRNA of the multiple copies of snRNA or/or multiple distinct snRNA is separated by a nucleic acid buffer sequence derived from human non-coding genomic sequences downstream of an snRNA. In one embodiment, the buffer sequence is derived from human genomic sequences downstream of U7.

In one embodiment, the buffer sequence is one of the following nucleic acid sequences:

buffer 1 (30 bp):
(SEQ ID NO: 118)
CAAACTACAGAGCCAAGTGCTATCCACAGA,
buffer 2 (30 bp):
(SEQ ID NO: 119)
GAGCTTTCTGGGTTGCCATCTCAAGCAGAC,
buffer 3 (30 bp):
(SEQ ID NO: 120)
TACAAGGCCATCAGCTCATACTCACAATTG,

or
a combination thereof.

In another embodiment, the buffer sequence is one of the following nucleic acid sequences:

buffer 1 (100 bp):
(SEQ ID NO: 121)
CAAACTACAGAGCCAAGTGCTATCCACAGAGAGCTTTCTGGGTTGCCATC
TCAAGCAGACTACAAGGCCATCAGCTCATACTCACAATTGACTTTGAGA
G,
buffer 2 (100 bp):
(SEQ ID NO: 122)
TTGACCACATACGTGCTCTTTCAAAGTTCTGTGTTTGAAGTTATGTTAGT
AACAACTGATGCCCATCCTGCAATGACAAATCCAATTCTCAGTGCAGCT
C,

a combination thereof.

In another embodiment, the buffer sequence is one of the following nucleic acid sequences:

buffer 1 (500 bp):
(SEQ ID NO: 123)
CAAACTACAGAGCCAAGTGCTATCCACAGAGAGCTTTCTGGGTTGCCATC
TCAAGCAGACTACAAGGCCATCAGCTCATACTCACAATTGACTTTGAGAG
TCATTTTCCAATGCTCCTACACACCCCTTCTTCACAATCCCCAACAAATC
TGAGGCTGGAACTTGGTACCATAACAATCATTACATTATTTCACCAGAAG
TACACCTTGCCTGGAAGATTGGCATTATAGCATCTTCTAACATTGTGAAA
GTTAGTGACCAATGAGGAGATCCAAGTCAGTTCCAGTTGGATTTCTCTAT
ACTCTATAATAAATATATATGGTGTCTTCAACAATAGGACTTTGCCATCC
AGTGATGCTAAAAATCAATAACAATGGCAATAACCTGCCCTGTTTGGAAA
GCCTCTGGCTTCCATGACTAACAATTCAAGGCAGGTCTCCTATACCTAGT
ACTGAGATTTTTATTTGATAAACTATATCTTCTGGGAGGAGAAGCATTG
T,
buffer 2 (500 bp):
(SEQ ID NO: 124)
TTGACCACATACGTGCTCTTTCAAAGTTCTGTGTTTGAAGTTATGTTAGT
AACAACTGATGCCCATCCTGCAATGACAAATCCAATTCTCAGTGCAGCTC
TCTGAAATAGTTTTGCTTTCTCTCTCTAGGTCTGTTCTATACTCCTAACT
CTCCAGGAGTTTACAAGGAATAAAATCTCTTCCAAATGCTTTCTGTTGCA
ACAACTGGACCATACTGAAAGCTGAGGCCCACAATTGCAATCTAGGTTAG
CAGGTAATCATTGTTGGTGAGGTCCTCCCTTTCCCCAGGCTCGTGTTTGT
ATTGGGGAGCAGGAAATTTTTGCTAGAGCAGCACTGCCATCTCTCTACAC
TCCACCTGATTGGTGGGATGGACCAGAGAAATGGACATTCCCAACACAGT
CCCTCCTTTCACATCTGCTCACCTGCCCACAGGATACTTTCCACCATGCA
TACTGGGCTCTGCACCAACCATTCAGCAGTGATGAAGAGGAAACTTGAA
C,

a combination thereof.

In some embodiments, the buffer sequence comprises, consists of, or consists essentially of the sequence selected from the group consisting of SEQ ID NOS: 118-124, or a sequence having 1, 2, 3, 4, 5, 6, 7, 8, 9 or 10 insertions, substitutions or deletions relative thereto. In some embodiments, the buffer sequence comprises, consists of, or consists essentially of the sequence selected from the group consisting of SEQ ID NOS: 118-124, or a sequence having at least about 70%, at least about 80%, at least about 90%, at least about 95%, or at least about 99% identity thereto.

The 100 bp and 500 bp buffer 1 sequences are derived from a sequence starting 100 bp downstream of the Mus musculus U7 pseudogene 8 (Location Chromosome 14: 4,409,359-4,409,421 reverse strand. GRCm39:CM001007.3). The 100 bp and 500 bp buffer 2 sequences are derived from the sequence starting 130 bp downstream of human U7 pseudogene 5 (Chromosome X: 140,451,148-140,451,208 forward strand.GRCh38:CM000685.2). Both 100 bp buffers are the first 100 bp of the corresponding 500 bp buffer (e.g., “buffer 1 (100 bp)” consists of the first 100 bp of “buffer 1 (500 bp”). The 30 bp buffers 1, 2, and 3, are sequential 30 bp sequences within “100 bp buffer 1”, downstream of the Mus musculus U7 pseudogene 8. These downstream sequences were selected due to the lack of any known regulatory sites or genes within or nearby to the sequence (using Gencode/Ensembl), in addition to lack of repetitive sequence, 40-60% GC content for total buffer, 40-60% GC content in the 20 bp region at both ends of the buffer, and minimal sequence complexity.

snRNA Sequences

The snRNA sequences of the disclosure can comprise any combination of esnRNA or snRNA features described herein.

In some embodiments, the snRNA comprises a targeting sequence that binds an SOD1 RNA sequence and a SL. In some embodiments, the SOD1 targeting sequence comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 1-37. In some embodiments, the SL comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 51-91. In some embodiments, the SL comprises or consists of the nucleic acid sequence set forth in SEQ ID NO: 64 or SEQ ID NO: 66.

In some embodiments, the snRNA comprises a targeting sequence that binds an SOD1 RNA sequence comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 1-37, and a SL comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 51-91. In some embodiments, the snRNA comprises a targeting sequence that binds an SOD1 RNA sequence comprising the nucleic acid sequence set forth in any one of SEQ ID NOs: 1-37, and the SL comprises the nucleic acid sequence set forth in SEQ ID NO: 64 or SEQ ID NO: 66.

In some embodiments, the SOD1 targeting sequence is positioned 5′ of the SL.

### Polynucleotides and Vectors

Also provided herein are polynucleotides and vectors (e.g., recombinant expression vectors) comprising the snRNA targeting SOD1.

In some embodiments of the compositions and methods of the disclosure, a polynucleotides or vector comprises or encodes an snRNA system targeting SOD1 provided herein. In some embodiments, the vector is a single or unitary vector.

Provided herein is a polynucleotide or vector comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 38-50. In some embodiments, the polynucleotide or vector comprises the nucleic acid sequence set forth in SEQ ID NO: 38. In some embodiments, the polynucleotide or vector comprises the nucleic acid sequence set forth in SEQ ID NO: 39. In some embodiments, the polynucleotide or vector comprises the nucleic acid sequence set forth in SEQ ID NO: 40. In some embodiments, the polynucleotide or vector comprises the nucleic acid sequence set forth in SEQ ID NO: 41. In some embodiments, the polynucleotide or vector comprises the nucleic acid sequence set forth in SEQ ID NO: 42. In some embodiments, the polynucleotide or vector comprises the nucleic acid sequence set forth in SEQ ID NO: 43. In some embodiments, the polynucleotide or vector comprises the nucleic acid sequence set forth in SEQ ID NO: 44. In some embodiments, the polynucleotide or vector comprises the nucleic acid sequence set forth in SEQ ID NO: 45. In some embodiments, the polynucleotide or vector comprises the nucleic acid sequence set forth in SEQ ID NO: 46. In some embodiments, the polynucleotide or vector comprises the nucleic acid sequence set forth in SEQ ID NO: 47. In some embodiments, the polynucleotide or vector comprises the nucleic acid sequence set forth in SEQ ID NO: 48. In some embodiments, the polynucleotide or vector comprises the nucleic acid sequence set forth in SEQ ID NO: 49. In some embodiments, the polynucleotide or vector comprises the nucleic acid sequence set forth in SEQ ID NO: 50.

In some embodiments, the polynucleotide or vector comprises the nucleic acid sequence set forth in any one of SEQ ID NOS: 38-50, or a sequence having 1, 2, 3, 4 or 5 insertions, deletions or substitutions relative thereto.

In some embodiments, snRNA system is capable of targeting one or more SOD1 RNA sequences. In some aspects, the SOD1 RNA sequence is a SOD1 pre-mRNA sequence. In some aspects, the snRNA systems are capable of targeting multiple (i.e., two or more) RNAs of interest. In some embodiments, the two or more RNAs of interest can be within the same pre-mRNA molecule, but different sequences within the pre-mRNA molecule, e.g. different exons, exon and untranslated region, and the like.

One type of vector is a “plasmid,” which refers to a circular double stranded DNA loop into which DNA segments in addition to the nucleotide of interest can be inserted, such as by standard molecular cloning techniques. Another type of vector is a viral vector, wherein virally derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentiviral (such as an integration-deficient lentiviral vector) or adeno-associated viral (AAV) vector. Vectors may be capable of autonomous replication in a host cell into which they are introduced such as e.g., bacterial vectors having a bacterial origin of replication and episomal mammalian vectors and other vectors such as, e.g., non-episomal mammalian vectors, are integrated into the genome of a host cell upon introduction into the host cell, and thereby are replicated along with the host genome.

In some embodiments, vectors such as e.g., expression vectors, are capable of directing the expression of genes they contain. Common expression vectors are often in the form of plasmids. In some embodiments, recombinant expression vectors comprise or encode a nucleic acid provided herein such as e.g., an snRNA or esnRNA in a form suitable for expression of an RNA molecule in a host cell. Recombinant expression vectors can include one or more regulatory elements, which may be selected on the basis of the host cells to be used for expression, that is operatively linked to the nucleic acid sequence to be expressed. Within a recombinant expression vector, “operably linked” is intended to mean that the nucleotide sequence of interest is linked to the regulatory element(s) in a manner that allows for expression of the nucleotide sequence such as e.g., in an in vitro transcription/translation system or in a host cell when the vector is introduced into the host cell. In some embodiments, the regulatory element is a promoter described herein. In some embodiments, the regulatory element is a terminator provided herein.

Certain embodiments of a vector depend on factors such as the choice of the host cell to be transformed, and the level of expression desired. A vector can be introduced into host cells to thereby produce transcripts, proteins, or peptides, including fusion proteins or peptides, encoded by nucleic acids as described herein such as, e.g., snRNAs, CRISPR transcripts, proteins, enzymes, mutant forms thereof, fusion proteins thereof, etc.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. An “enhancer” is a region of DNA that can be bound by activating proteins to increase the likelihood or frequency of transcription.

In some embodiments of the compositions and methods of the disclosure, an expression vector, viral vector or non-viral vector provided herein, includes without limitation, vector elements such as a buffer sequence derived human genomic sequences downstream from an snRNA and as such will have the capability to encoding multiple snRNAs from a single construct.

In some embodiments, the snRNA constructs disclosed herein comprise bidirectional snRNA promoters to express snRNAs.

In another embodiment, the vector configurations can comprise linker(s), signal sequence(s), and/or tag(s).

### Viral Vectors

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors.

In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV2-Tyr mutant vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAVrh8 vector, an AAV9 vector, an AAV.rh10 vector, a modified AAV.rh10 vector, an AAVrh.74, an AAV.rh32/33 vector, a modified AAV.rh32/33 vector, an AAV.rh43 vector, a modified AAV.rh43 vector, an AAV.rh64R1 vector, and a modified AAV.rh64R1 vector, an AAV-Tyr mutant vector, AAV-Tyr-Ser mutant vector, AAV-Tyr-Ser-Thr mutant vector and any combinations or equivalents thereof.

In some embodiments, the vector is a lentiviral vector. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism.

In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments, the vector further comprises one or more expression control elements operably linked to the polynucleotide comprising or encoding the snRNA or esnRNA described herein. In some embodiments, the vector further comprises one or more selectable markers. In some embodiments, the vector has low toxicity. In some embodiments, the vector does not incorporate into the host genome, thereby having a low probability of causing insertional mutagenesis.

### Lentiviral Vectors

Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Adeno-Associated Virus Vectors

In some aspect, a vector described herein is an AAV viral vector. The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (560 to 65 °C for several hours), making cold preservation of AAV less critical. AAV may even be lyophilized. Finally, AAV-infected cells are not resistant to superinfection.

Recombinant AAV (rAAV) genomes of the invention may comprise, consist essentially of, or consist of a nucleic acid molecule encoding at least one snRNA or esnRNA and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

An AAV vector described herein may comprise, consist essentially of, or consist of one or more nucleic acid molecules and one or more AAV ITRs. In some aspects, the nucleic acid molecule encodes an esnRNA of the disclosure. Such AAV vectors can be replicated and packaged into infectious viral particles when present in a host cell that provides the functionality of rep and cap gene products, for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vectors may also contain elements for manufacturing purposes, e.g., antibiotic resistance genes, origin of replication sequences etc., but these are not encapsidated and thus do not form part of the AAV particle.

In some aspects, an AAV vector can comprise at least one nucleic acid encoding an snRNA or esnRNA of the disclosure. In some aspects, an AAV vector can comprise at least one regulatory sequence. In some aspects, an AAV vector can comprise at least one AAV inverted terminal (ITR) sequence. In some aspects, an AAV vector can comprise a first ITR sequence and a second ITR sequence. In some aspects, an AAV vector can comprise at least one promoter sequence. In some aspects, an AAV vector can comprise at least one enhancer sequence. In some aspects, an AAV vector can comprise at least one terminator sequence. In some aspects, an AAV vector can comprise at least one polyA sequence. In some aspects, an AAV vector can comprise at least one linker sequence. In some aspects, an AAV vector can comprise at least one buffer sequence. In some aspects, an AAV vector of the disclosure can comprise at least one nuclear localization signal, or nuclear export signal and/or both.

In some aspects, an AAV vector can comprise a first AAV ITR sequence, a promoter sequence, an snRNA sequence and/or esnRNA sequence, a terminator sequence and a second AAV ITR sequence. In some aspects, an AAV vector can comprise, in the 5′ to 3′ direction, a first AAV ITR sequence, a promoter sequence, an esnRNA sequence, a terminator sequence, and a second AAV ITR sequence. In some aspects, an AAV vector can comprise, in the 5′ to 3′ direction, a first AAV ITR sequence, a promoter sequence, an snRNA sequence, a terminator sequence, and a second AAV ITR sequence. Exemplary ITR sequences are provided as SEQ ID NOS: 125 and 126, and additional suitable ITR sequences will be known to persons of ordinary skill in the art.

In some aspects, an AAV vector can comprise a first AAV ITR sequence, a first promoter sequence, a first snRNA sequence, a termination sequence, a second promoter sequence, second snRNA sequence, a second termination sequence and a second AAV ITR sequence. In some aspects, an AAV vector can comprise a first AAV ITR sequence, a first promoter sequence, a first snRNA sequence, a termination sequence, a second promoter sequence, a second snRNA sequence, a second termination sequence, a third promoter sequence, a third snRNA sequence, a third termination sequence, and a second AAV ITR sequence. In some aspects, an AAV vector can comprise a first AAV ITR sequence, a first promoter sequence, a first snRNA sequence, a termination, a second promoter sequence, a second snRNA sequence, a second termination sequence, a third promoter sequence, a third snRNA sequence, a third termination sequence, a fourth promoter sequence, a fourth snRNA sequence, a fourth termination sequence, and a second AAV ITR sequence. The packaging of multiple snRNA or other repetitive elements is described in more detail in International Patent Application Publication No. WO2024119102A1, which is incorporated herein by reference in its entirety.

In some embodiments of the compositions and methods of the disclosure, the viral vector comprises a sequence isolated or derived from an adeno-associated virus (AAV). In some embodiments, the viral vector comprises an ITR sequence or a capsid sequence that is isolated or derived from an AAV of serotype AAV1, AAV2, AAV3, AAV4, AAV5, AAV6, AAV7, AAV8, AAV9, AAV10, AAVrh10, AAV11 or AAV12. In some embodiments, the AAV serotype is AAVrh.74. In one embodiment, the AAV vector comprises a modified capsid. In one embodiment the AAV vector is an AAV2-Tyr mutant vector. In one embodiment the AAV vector comprises a capsid with a non-tyrosine amino acid at a position that corresponds to a surface-exposed tyrosine residue in position Tyr252, Tyr272, Tyr275, Tyr281, Tyr508, Tyr612, Tyr704, Tyr720, Tyr730 or Tyr673 of wild-type AAV2. See also WO 2008/124724 incorporated herein in its entirety by reference. In some embodiments, the AAV vector comprises an engineered capsid. AAV vectors comprising engineered capsids include without limitation, AAV2.7m8, AAV9.7m8, AAV2 2tYF, and AAV8 Y733F). In some embodiments, the capsid is a ubiquitination resistant capsid. In another embodiment, the ubiquitination capsid is an AAV2 capsid comprising tyrosine (Y) and serine (S) mutations. In another embodiment, the AAV2 capsid comprises Y, S and threonine (T) mutations. In another embodiment, the AAV2 capsid includes, without limitation, AAV2 capsid mutants such as T455V, T491V, T550V, T659V, Y444+500+730F, and/or Y444+500+730F+T491V. In some embodiments, the viral vector is replication incompetent. In some embodiments, the viral vector is isolated or recombinant (rAAV). In some embodiments, the viral vector is self-complementary (scAAV). In some embodiments, the viral vector is single-stranded (ssAAV).

In some embodiments, the sequences encoding the snRNAs provided herein are comprised within a single-stranded AAV (ssAAV). In some embodiments, the sequences encoding the snRNAs provided herein are comprised within a self-complementary AAV (scAAV). The single-stranded nature of the parvoviral genome requires the use of cellular mechanisms to provide a complementary-strand for gene expression. This cellular recruitment activity is considered a rate-limiting factor in the efficiency of transduction and gene expression in parvoviruses and parvoviral particles. The use of an scAAV versus an ssAAV remedies this well-known issue by packaging both strands as a single duplex DNA molecule (or inverted repeat genome) that can fold into dsDNA as a result of a self-complementary viral genome sequence. In this regard, the requirement for DNA synthesis or base-pairing between multiple viral genomes is eliminated.

### AAV ITR Sequences

In some embodiments of the compositions and methods of the disclosure, an AAV inverted terminal repeat sequence can comprise any AAV ITR sequence known in the art. In some aspects, an AAV ITR sequence can comprise or consist of an AAV1 ITR sequence, an AAV2 ITR sequence, an AAV3 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAVrh10 ITR sequence, an AAV11 ITR sequence, an AAV12 ITR sequence, an AAV13 ITR sequence, or an AAVrh74 ITR sequence.

In some aspects the ITR sequence can comprise a modified AAV ITR sequence.

In some aspects, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 125 or SEQ ID NO: 126. In some aspects, an AAV ITR sequence can comprise, consist essentially of, or consist of the nucleic acid sequence of SEQ ID NO: 125 or 126. In some aspects, an AAV ITR sequence can comprise, consist essentially of, or consist of the nucleic acid sequence of SEQ ID NO: 125 or 126, or a sequence having 1, 2, 3, 4 or 5 insertions, deletions or substitutions relative thereto.

In some embodiments, an AAV vector provided herein comprises a first and a second AAV ITR sequence. In some aspects, a first AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 125 or SEQ ID NO: 126 and a second AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 125 or SEQ ID NO: 126. In some aspects the first AAV ITR sequence is positioned at the 5′ of an AAV vector. In some aspects the second AAV ITR sequence is positioned at the 3′ of an AAV vector.

In some aspects, a first AAV ITR sequence comprises the sequence set forth in SEQ ID NO: 125 or SEQ ID NO: 126. In some aspects, a second AAV ITR sequence comprises the sequence set forth in SEQ ID NO: 125 or SEQ ID NO: 126. In some embodiments, an AAV vector provided herein comprises a first AAV ITR sequence comprising the sequence set forth in SEQ ID NO: 125 and a second AAV ITR sequence comprising the sequence set forth in SEQ ID NO: 126. In some aspects the first AAV ITR sequence is positioned at the 5′ of an AAV vector. In some aspects the second AAV ITR sequence is positioned at the 3′ of an AAV vector.

In some embodiments of the compositions and methods of the disclosure, the viral vector comprises a sequence isolated or derived from an adeno-associated virus (AAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the nanoparticle comprises a lipid nanoparticle. In some embodiments, the vector is an expression vector or recombinant expression system. As used herein, the term “recombinant expression system” refers to a genetic construct for the expression of certain genetic material formed by recombination.

snRNA Vector Constructs

Also provided herein are vector constructs targeting SOD1 comprising the snRNA constructs described herein.

An illustrative AAV vector of the disclosure targeting SOD1 exon 2 is A04053. The elements of A04053 are set forth in Table. In some aspects a nucleic acid sequence encoding AAV vector A04053 comprises SEQ ID NO: 38.

TABLE 7
A04053 vector-scAAV-2x_mU7prom-SOD1z8/z4-mU7term_mU1prom-SOD1z8/z4-
mU1term; mouseloop with5′ ISD and eSL; Nucleotide sequences of plasmid elements in order
5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z8/z4 | CCATGCAGGCCTTCAGTCAGCCCACACCTTCACTGGT |
| | CCA SEQ ID NO: 18 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z8/z4 | CCATGCAGGCCTTCAGTCAGCCCACACCTTCACTGGT |
| | CCA SEQ ID NO: 18 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A04053 Nucleotide Sequence (whole transgene from ITR to ITR):

(SEQ ID NO: 38)
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaat
aaggaactgtgctttgtgattcacatatcagtggaggggtgtggaaatggcaccttgatctcaccctcatcgaaagtggagttgatgtcct
TccctggctcgctacagacgcacttccgcaaggagtCCATGCAGGCCTTCAGTCAGCCCACACCTTCACTGGTCC
AAATTTTTGGAGcaggttttctgacctccgtcggaaaacccccaatttcactggtctacaatgaaagcaaaacagttctcttccccg
ctccccggtgtgtgagaggggctttgatccttctctggtttcctaggaaacgcgtatgtgTTGTTCCTCTTAGTGTTAATTC
ACACTAAAGACTGTGCATCCGACTCCTACATTTATGAAAGTAAATGCCTGTTGTTAG
AACAAAAAAGGCTACAGAACAAAAAACAAAGCGAAATACCATCTGCTTTAGGTTCA
GTGTGGTATTTTCCCGCTGACAGGGAGGCGGGTTTTTGGGTACAGGAAACGAGTCAC
TATGGAGGCGGTACTATGTAGATGAGAATTCAGGTGCAAACTGGGAAAAGCAACTG
CTTCCAAATATTTGTGATTTTTACAGTGTAGTTTTGGAAAAACTCTTAGCCTACCAAT
TCTTCTAAGTGTTTTAAAATGTGGGAGCCAGTACACATGAAGTTATAGAGTGTTTTA
ATGAGGCTTAAATATTTACCGTAACTATGAAATGCTACGCATATCATGCTGTTCAGG
CTCCGTGGCCACGCAACTCggagtCCATGCAGGCCTTCAGTCAGCCCACACCTTCACTG
GTCCAAATTTTTGGAGcaggttttctgacctccgtcggaaaaccGTTTACTTGGTTTTAAAAATAGCT
TGCACTAGCGATACGGAATATGGTTATTAGGTTTGTTAGGCATCATGTCGTGTCTTA
CTATAGAAAAATAACGTAGTGTTCATTTTAGCCTGCCTGTATGTGTTAATTTGTCCTT
ATTGCGCATTGTTCTTGTTAAGTCTTCTGTAAGGAGTTGCGGGTTTCAAACTGTCAGT
CTGAGAGCAGAATTCGATATCTAGATCTCGAGGTAACCACGTGCGGACCCAACGGC
CGCaggaacccctagtgatggagttggccactccctctctgcgcgctcgctcgctcactgaggccgggcgaccaaaggtcgcccgacg
cccgggctttgcccgggcggcctcagtgagcgagcgagcgcgcagctgcctgcagg.

An illustrative AAV vector of the disclosure targeting SOD1 exon 2 is A04054. The elements of A04054 are set forth in Table. In some aspects a nucleic acid sequence encoding AAV vector A04054 comprises SEQ ID NO: 39.

TABLE 8
A04054 vector-scAAV-2x_mU7prom-SOD1z9/z2-mU7term_mU1prom-SOD1z9/z2-
mU1term; mouseloop with5′ ISD and eSL; Nucleotide sequences of plasmid elements in order
5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z9/z2 | GCTGTATTATCTCCAAACTCATGAACCACACCTTCAC |
| | TGGTCCATTACTTTCCTTT SEQ ID NO: 26 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z9/z2 | GCTGTATTATCTCCAAACTCATGAACCACACCTTCAC |
| | TGGTCCATTACTTTCCTTT SEQ ID NO: 26 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A04054 Nucleotide Sequence (whole transgene from ITR to ITR):

(SEQ ID NO: 39)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccgaa
taaggaactgtgctttgtgattcacatatcagtggaggggtgtggaaatggcaccttgatctcaccctcatcgaaagtggagttgatgtcc
tTccctggctcgctacagacgcacttccgcaaggagtGCTGTATTATCTCCAAACTCATGAACCACACCTTCACT
GGTCCATTACTTTCCTTTAATTTTTGGAGcaggttttctgacctccgtcggaaaacccccaatttcactggtctaca
atgaaagcaaaacagttctcttccccgctccccggtgtgtgagaggggctttgatccttctctggtttcctaggaaacgcgtatgtgTTG
TTCCTCTTAGTGTTAATTCACACTAAAGACTGTGCATCCGACTCCTACATTTATGAAA
GTAAATGCCTGTTGTTAGAACAAAAAAGGCTACAGAACAAAAAACAAAGCGAAAT
ACCATCTGCTTTAGGTTCAGTGTGGTATTTTCCCGCTGACAGGGAGGCGGGTTTTTG
GGTACAGGAAACGAGTCACTATGGAGGCGGTACTATGTAGATGAGAATTCAGGTGC
AAACTGGGAAAAGCAACTGCTTCCAAATATTTGTGATTTTTACAGTGTAGTTTTGGA
AAAACTCTTAGCCTACCAATTCTTCTAAGTGTTTTAAAATGTGGGAGCCAGTACACA
TGAAGTTATAGAGTGTTTTAATGAGGCTTAAATATTTACCGTAACTATGAAATGCTA
CGCATATCATGCTGTTCAGGCTCCGTGGCCACGCAACTCggagtGCTGTATTATCTCCA
AACTCATGAACCACACCTTCACTGGTCCATTACTTTCCTTTAATTTTTGGAGcaggttttctg
acctccgtcggaaaaccGTTTACTTGGTTTTAAAAATAGCTTGCACTAGCGATACGGAATATG
GTTATTAGGTTTGTTAGGCATCATGTCGTGTCTTACTATAGAAAAATAACGTAGTGT
TCATTTTAGCCTGCCTGTATGTGTTAATTTGTCCTTATTGCGCATTGTTCTTGTTAAGT
CTTCTGTAAGGAGTTGCGGGTTTCAAACTGTCAGTCTGAGAGCAGAATTCGATATCT
AGATCTCGAGGTAACCACGTGCGGACCCAACGGCCGCaggaacccctagtgatggagttggccactcc
ctctctgcgcgctcgctcgctcactgaggccgggcgaccaaaggtcgcccgacgcccgggctttgcccgggcggcctcagtgagcgag
cgagcgcgcagctgcctgcagg.

An illustrative AAV vector of the disclosure targeting SOD1 exon 2 is A04055. The elements of A04055 are set forth in 9. In some aspects a nucleic acid sequence encoding AAV vector A04055 comprises SEQ ID NO: 40.

TABLE 9
A04055 vector-scAAV-2x_mU7prom-SOD1z9/z3-mU7term_mU1prom-SOD1z9/z3-
mU1term; mouseloop with5′ ISD and eSL; Nucleotide sequences of plasmid elements in order
5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z9/z3 | GCTGTATTATCTCCAAACTCATGAACTTCCCCACACC |
| | TTCACTGGTCCATTA SEQ ID NO: 28 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z9/z3 | GCTGTATTATCTCCAAACTCATGAACTTCCCCACACC |
| | TTCACTGGTCCATTA SEQ ID NO: 28 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A04055 Nucleotide Sequence (whole transgene from ITR to ITR):

(SEQ ID NO: 40)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccgaa
taaggaactgtgctttgtgattcacatatcagtggaggggtgtggaaatggcaccttgatctcaccctcatcgaaagtggagttgatgtcc
tTccctggctcgctacagacgcacttccgcaaggagtGCTGTATTATCTCCAAACTCATGAACTTCCCCACACCT
TCACTGGTCCATTAAATTTTTGGAGcaggttttctgacctccgtcggaaaacccccaatttcactggtctacaatgaaa
gcaaaacagttctcttccccgctccccggtgtgtgagaggggctttgatccttctctggtttcctaggaaacgcgtatgtgTTGTTCCT
CTTAGTGTTAATTCACACTAAAGACTGTGCATCCGACTCCTACATTTATGAAAGTAA
ATGCCTGTTGTTAGAACAAAAAAGGCTACAGAACAAAAAACAAAGCGAAATACCAT
CTGCTTTAGGTTCAGTGTGGTATTTTCCCGCTGACAGGGAGGCGGGTTTTTGGGTAC
AGGAAACGAGTCACTATGGAGGCGGTACTATGTAGATGAGAATTCAGGTGCAAACT
GGGAAAAGCAACTGCTTCCAAATATTTGTGATTTTTACAGTGTAGTTTTGGAAAAAC
TCTTAGCCTACCAATTCTTCTAAGTGTTTTAAAATGTGGGAGCCAGTACACATGAAG
TTATAGAGTGTTTTAATGAGGCTTAAATATTTACCGTAACTATGAAATGCTACGCAT
ATCATGCTGTTCAGGCTCCGTGGCCACGCAACTCggagtGCTGTATTATCTCCAAACTC
ATGAACTTCCCCACACCTTCACTGGTCCATTAAATTTTTGGAGcaggttttctgacctccgtcggaa
aaccGTTTACTTGGTTTTAAAAATAGCTTGCACTAGCGATACGGAATATGGTTATTAGG
TTTGTTAGGCATCATGTCGTGTCTTACTATAGAAAAATAACGTAGTGTTCATTTTAGC
CTGCCTGTATGTGTTAATTTGTCCTTATTGCGCATTGTTCTTGTTAAGTCTTCTGTAAG
GAGTTGCGGGTTTCAAACTGTCAGTCTGAGAGCAGAATTCGATATCTAGATCTCGAG
GTAACCACGTGCGGACCCAACGGCCGCaggaacccctagtgatggagttggccactccctctctgcgcgctcgct
cgctcactgaggccgggcgaccaaaggtcgcccgacgcccgggctttgcccgggcggcctcagtgagcgagcgagcgcgcagctgc
ctgcagg.

An illustrative AAV vector of the disclosure targeting SOD1 exon 2 is A05204. The elements of A05204 are set forth in 10. In some aspects a nucleic acid sequence encoding AAV vector A05204 comprises SEQ ID NO: 41.

TABLE 10
A05204 vector-scAAV-2x_mU7prom-SOD1z3/z10-mU7term_mU1prom-
SOD1z3/z10-mU1term; mouseloop with5′ ISD and eSL; Nucleotide sequences of plasmid
elements in order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z3/z10 | TTCCCCACACCTTCACTGGTCCATTAACCTGCTGTAT |
| | TATCTCCAAACTCATGAAC SEQ ID NO: 19 |
| SMOPT | AATTTTTGGAGca SEQ ID NO: 127 |
| Stem loop | ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z3/z10 | TTCCCCACACCTTCACTGGTCCATTAACCTGCTGTAT |
| | TATCTCCAAACTCATGAAC SEQ ID NO: 19 |
| SMOPT | AATTTTTGGAGca SEQ ID NO: 127 |
| Stem loop | Ggttttctgacctccgtcggaaaacc SEQ ID NO: 66 |
| Mouse Ul terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A05204 Nucleotide Sequence (whole transgene from ITR to ITR):

(SEQ ID NO: 41)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccgaa
taaggaactgtgctttgtgattcacatatcagtggaggggtgtggaaatggcaccttgatctcaccctcatcgaaagtggagttgatgtcc
tTccctggctcgctacagacgcacttccgcaaggagtTTCCCCACACCTTCACTGGTCCATTAACCTGCTGTATTA
TCTCCAAACTCATGAACAATTTTTGGAGcaggttttctgacctccgtcggaaaacccccaatttcactggtctacaa
tgaaagcaaaacagttctcttccccgctccccggtgtgtgagaggggctttgatccttctctggtttcctaggaaacgcgtatgtgTTGT
TCCTCTTAGTGTTAATTCACACTAAAGACTGTGCATCCGACTCCTACATTTATGAAA
GTAAATGCCTGTTGTTAGAACAAAAAAGGCTACAGAACAAAAAACAAAGCGAAAT
ACCATCTGCTTTAGGTTCAGTGTGGTATTTTCCCGCTGACAGGGAGGCGGGTTTTTG
GGTACAGGAAACGAGTCACTATGGAGGCGGTACTATGTAGATGAGAATTCAGGTGC
AAACTGGGAAAAGCAACTGCTTCCAAATATTTGTGATTTTTACAGTGTAGTTTTGGA
AAAACTCTTAGCCTACCAATTCTTCTAAGTGTTTTAAAATGTGGGAGCCAGTACACA
TGAAGTTATAGAGTGTTTTAATGAGGCTTAAATATTTACCGTAACTATGAAATGCTA
CGCATATCATGCTGTTCAGGCTCCGTGGCCACGCAACTCggagtTTCCCCACACCTTCA
CTGGTCCATTAACCTGCTGTATTATCTCCAAACTCATGAACAATTTTTGGAGcaggttttct
gacctccgtcggaaaaccGTTTACTTGGTTTTAAAAATAGCTTGCACTAGCGATACGGAATAT
GGTTATTAGGTTTGTTAGGCATCATGTCGTGTCTTACTATAGAAAAATAACGTAGTG
TTCATTTTAGCCTGCCTGTATGTGTTAATTTGTCCTTATTGCGCATTGTTCTTGTTAAG
TCTTCTGTAAGGAGTTGCGGGTTTCAAACTGTCAGTCTGAGAGCAGAATTCGATATC
TAGATCTCGAGGTAACCACGTGCGGACCCAACGGCCGCaggaacccctagtgatggagttggccact
ccctctctgcgcgctcgctcgctcactgaggccgggcgaccaaaggtcgcccgacgcccgggctttgcccgggcggcctcagtgagcg
agcgagcgcgcagctgcctgcagg.

An illustrative AAV vector of the disclosure targeting SOD1 exon 2 is A05205. The elements of A05205 are set forth in 11. In some aspects a nucleic acid sequence encoding AAV vector A05205 comprises SEQ ID NO: 42.

TABLE 11
A05205 vector-scAAV-2x_mU7prom-SOD1z2/z10-mU7term_mU1prom-SOD1z2/z10-
mU1term; mouseloop with5′ ISD and eSL; mouseloop with5′ ISD and eSL; Nucleotide
sequences of plasmid elements in order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z2/z10 | CACACCTTCACTGGTCCATTACTTTCCTTTACCTGCTG |
| | TATTATCTCCAAACTCATGAAC SEQ ID NO: 33 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z2/z10 | CACACCTTCACTGGTCCATTACTTTCCTTTACCTGCTG |
| | TATTATCTCCAAACTCATGAAC SEQ ID NO: 33 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A05205 Nucleotide Sequence (whole transgene from ITR to ITR):

(SEQ ID NO: 42)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccgaata
aggaactgtgctttgtgattcacatatcagtggaggggtgtggaaatggcaccttgatctcaccctcatcgaaagtggagttgatgtcctTcc
ctggctcgctacagacgcacttccgcaaggagtCACACCTTCACTGGTCCATTACTTTCCTTTACCTGCTGT
ATTATCTCCAAACTCATGAACAATTTTTGGAGcaggttttctgacctccgtcggaaaacccccaatttcactgg
tctacaatgaaagcaaaacagttctcttccccgctccccggtgtgtgagaggggctttgatccttctctggtttcctaggaaacgcgtatgt
gTTGTTCCTCTTAGTGTTAATTCACACTAAAGACTGTGCATCCGACTCCTACATTTATGA
AAGTAAATGCCTGTTGTTAGAACAAAAAAGGCTACAGAACAAAAAACAAAGCGAA
ATACCATCTGCTTTAGGTTCAGTGTGGTATTTTCCCGCTGACAGGGAGGCGGGTTTTT
GGGTACAGGAAACGAGTCACTATGGAGGCGGTACTATGTAGATGAGAATTCAGGTG
CAAACTGGGAAAAGCAACTGCTTCCAAATATTTGTGATTTTTACAGTGTAGTTTTGG
AAAAACTCTTAGCCTACCAATTCTTCTAAGTGTTTTAAAATGTGGGAGCCAGTACAC
ATGAAGTTATAGAGTGTTTTAATGAGGCTTAAATATTTACCGTAACTATGAAATGCT
ACGCATATCATGCTGTTCAGGCTCCGTGGCCACGCAACTCggagtCACACCTTCACTGG
TCCATTACTTTCCTTTACCTGCTGTATTATCTCCAAACTCATGAACAATTTTTGGAGca
ggttttctgacctccgtcggaaaaccGTTTACTTGGTTTTAAAAATAGCTTGCACTAGCGATACGGA
ATATGGTTATTAGGTTTGTTAGGCATCATGTCGTGTCTTACTATAGAAAAATAACGT
AGTGTTCATTTTAGCCTGCCTGTATGTGTTAATTTGTCCTTATTGCGCATTGTTCTTGT
TAAGTCTTCTGTAAGGAGTTGCGGGTTTCAAACTGTCAGTCTGAGAGCAGAATTCGA
TATCTAGATCTCGAGGTAACCACGTGCGGACCCAACGGCCGCaggaacccctagtgatggagttg
gccactccctctctgcgcgctcgctcgctcactgaggccgggcgaccaaaggtcgcccgacgcccgggctttgcccgggcggcctcagt
gagcgagcgagcgcgcagctgcctgcagg.

An illustrative AAV vector of the disclosure targeting SOD1 exon 2 is A05206. The elements of A05206 are set forth in 12. In some aspects a nucleic acid sequence encoding AAV vector A05206 comprises SEQ ID NO: 43.

TABLE 12
A05206 vector-scAAV-2x_mU7prom-SOD1z10/z2-mU7term_mU1prom-SOD1z10/z2-
mU1term; mouseloop with5′ ISD and eSL; Nucleotide sequences of plasmid elements in order
5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z10/z2 | ACCTGCTGTATTATCTCCAAACTCATGAACCACACCT |
| | TCACTGGTCCATTACTTTCCTTT SEQ ID NO: 34 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z10/z2 | ACCTGCTGTATTATCTCCAAACTCATGAACCACACCT |
| | TCACTGGTCCATTACTTTCCTTT SEQ ID NO: 34 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A05206 Nucleotide Sequence (whole transgene from ITR to ITR):

(SEQ ID NO: 43)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccgaa
taaggaactgtgctttgtgattcacatatcagtggaggggtgtggaaatggcaccttgatctcaccctcatcgaaagtggagttgatgtcc
tTccctggctcgctacagacgcacttccgcaaggagtACCTGCTGTATTATCTCCAAACTCATGAACCACACCTT
CACTGGTCCATTACTTTCCTTTAATTTTTGGAGcaggttttctgacctccgteggaaaacccccaatttcactg
gtctacaatgaaagcaaaacagttctcttccccgctccccggtgtgtgagaggggctttgatccttctctggtttcctaggaaacgcgtat
gtgTTGTTCCTCTTAGTGTTAATTCACACTAAAGACTGTGCATCCGACTCCTACATTTAT
GAAAGTAAATGCCTGTTGTTAGAACAAAAAAGGCTACAGAACAAAAAACAAAGCG
AAATACCATCTGCTTTAGGTTCAGTGTGGTATTTTCCCGCTGACAGGGAGGCGGGTT
TTTGGGTACAGGAAACGAGTCACTATGGAGGCGGTACTATGTAGATGAGAATTCAG
GTGCAAACTGGGAAAAGCAACTGCTTCCAAATATTTGTGATTTTTACAGTGTAGTTT
TGGAAAAACTCTTAGCCTACCAATTCTTCTAAGTGTTTTAAAATGTGGGAGCCAGTA
CACATGAAGTTATAGAGTGTTTTAATGAGGCTTAAATATTTACCGTAACTATGAAAT
GCTACGCATATCATGCTGTTCAGGCTCCGTGGCCACGCAACTCggagtACCTGCTGTAT
TATCTCCAAACTCATGAACCACACCTTCACTGGTCCATTACTTTCCTTTAATTTTTGG
AGcaggttttctgacctccgtcggaaaaccGTTTACTTGGTTTTAAAAATAGCTTGCACTAGCGATAC
GGAATATGGTTATTAGGTTTGTTAGGCATCATGTCGTGTCTTACTATAGAAAAATAA
CGTAGTGTTCATTTTAGCCTGCCTGTATGTGTTAATTTGTCCTTATTGCGCATTGTTCT
TGTTAAGTCTTCTGTAAGGAGTTGCGGGTTTCAAACTGTCAGTCTGAGAGCAGAATT
CGATATCTAGATCTCGAGGTAACCACGTGCGGACCCAACGGCCGCaggaacccctagtgatgg
agttggccactccctctctgcgcgctcgctcgctcactgaggccgggcgaccaaaggtcgcccgacgcccgggctttgcccgggcggcc
tcagtgagcgagcgagcgcgcagctgcctgcagg.

An illustrative AAV vector of the disclosure targeting SOD1 exon 2 is A05207. The elements of A05207 are set forth in 13. In some aspects a nucleic acid sequence encoding AAV vector A05207 comprises SEQ ID NO: 44.

TABLE 13
A05207 vector scAAV-2x_mU7prom-SOD1z11/z12-mU7term_mU1prom-
SOD1z11/z12-mU1term; mouseloop with5′ ISD and eSL; Nucleotide sequences of plasmid
elements in order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z11/z12 | CACACCTTCACTGGTCCATTACTTTCCTTTAAGAAAA |
| | CACCCACCTGCTGTATTATCTCCAAACTCATGAAC |
| | SEQ ID NO: 29 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z11/z12 | CACACCTTCACTGGTCCATTACTTTCCTTTAAGAAAA |
| | CACCCACCTGCTGTATTATCTCCAAACTCATGAAC |
| | SEQ ID NO: 29 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A05207 Nucleotide Sequence (whole transgene from ITR to ITR):

(SEQ ID NO: 44)
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaa
taaggaactgtgctttgtgattcacatatcagtggaggggtgtggaaatggcaccttgatctcaccctcatcgaaagtggagttgatgtc
ctTccctggctcgctacagacgcacttccgcaaggagtCACACCTTCACTGGTCCATTACTTTCCTTTAAGAAAACA
CCCACCTGCTGTATTATCTCCAAACTCATGAACAATTTTTGGAGcaggttttctgacctccgtcgga
aaacccccaatttcactggtctacaatgaaagcaaaacagttctcttccccgctccccggtgtgtgagaggggctttgatccttctctgg
tttcctaggaaacgcgtatgtgTTGTTCCTCTTAGTGTTAATTCACACTAAAGACTGTGCATCCGACTC
CTACATTTATGAAAGTAAATGCCTGTTGTTAGAACAAAAAAGGCTACAGAACAAAA
AACAAAGCGAAATACCATCTGCTTTAGGTTCAGTGTGGTATTTTCCCGCTGACAGGG
AGGCGGGTTTTTGGGTACAGGAAACGAGTCACTATGGAGGCGGTACTATGTAGATG
AGAATTCAGGTGCAAACTGGGAAAAGCAACTGCTTCCAAATATTTGTGATTTTTACA
GTGTAGTTTTGGAAAAACTCTTAGCCTACCAATTCTTCTAAGTGTTTTAAAATGTGG
GAGCCAGTACACATGAAGTTATAGAGTGTTTTAATGAGGCTTAAATATTTACCGTAA
CTATGAAATGCTACGCATATCATGCTGTTCAGGCTCCGTGGCCACGCAACTCggagtCA
CACCTTCACTGGTCCATTACTTTCCTTTAAGAAAACACCCACCTGCTGTATTATCTCC
AAACTCATGAACAATTTTTGGAGcaggttttctgacctccgtcggaaaaccGTTTACTTGGTTTTAAA
AATAGCTTGCACTAGCGATACGGAATATGGTTATTAGGTTTGTTAGGCATCATGTCG
TGTCTTACTATAGAAAAATAACGTAGTGTTCATTTTAGCCTGCCTGTATGTGTTAATT
TGTCCTTATTGCGCATTGTTCTTGTTAAGTCTTCTGTAAGGAGTTGCGGGTTTCAAAC
TGTCAGTCTGAGAGCAGAATTCGATATCTAGATCTCGAGGTAACCACGTGCGGACCC
AACGGCCGCaggaacccctagtgatggagttggccactccctctctgcgcgctcgctcgctcactgaggccgggcgaccaaagg
tcgcccgacgcccgggctttgcccgggcggcctcagtgagcgagcgagcgcgcagctgcctgcagg.

An illustrative AAV vector of the disclosure targeting SOD1 exon 2 is A05208. The elements of A05208 are set forth in 14. In some aspects a nucleic acid sequence encoding AAV vector A05208 comprises SEQ ID NO: 45.

TABLE 14
A05208 vector-scAAV-2x_mU7prom-SOD1z12/z11-mU7term_mU1prom-
SOD1z12/z11-mU1term; mouseloop with5′ ISD and eSL; Nucleotide sequences of plasmid
elements in order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z12/z11 | ACACCCACCTGCTGTATTATCTCCAAACTCATGAACC |
| | ACACCTTCACTGGTCCATTACTTTCCTTTAAGAAA |
| | SEQ ID NO: 30 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z12/z11 | ACACCCACCTGCTGTATTATCTCCAAACTCATGAACC |
| | ACACCTTCACTGGTCCATTACTTTCCTTTAAGAAA |
| | SEQ ID NO: 30 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A05208 Nucleotide Sequence (whole transgene from ITR to ITR):

(SEQ ID NO: 45)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccga
ataaggaactgtgctttgtgattcacatatcagtggaggggtgtggaaatggcaccttgatctcaccctcatcgaaagtggagttgatgt
cctTccctggctcgctacagacgcacttccgcaaggagtACACCCACCTGCTGTATTATCTCCAAACTCATGAACCA
CACCTTCACTGGTCCATTACTTTCCTTTAAGAAAAATTTTTGGAGcaggttttctgacctccgtcg
gaaaacccccaatttcactggtctacaatgaaagcaaaacagttctcttccccgctccccggtgtgtgagaggggctttgatccttctct
ggtttcctaggaaacgcgtatgtgTTGTTCCTCTTAGTGTTAATTCACACTAAAGACTGTGCATCCGACT
CCTACATTTATGAAAGTAAATGCCTGTTGTTAGAACAAAAAAGGCTACAGAACAAA
AAACAAAGCGAAATACCATCTGCTTTAGGTTCAGTGTGGTATTTTCCCGCTGACAGG
GAGGCGGGTTTTTGGGTACAGGAAACGAGTCACTATGGAGGCGGTACTATGTAGAT
GAGAATTCAGGTGCAAACTGGGAAAAGCAACTGCTTCCAAATATTTGTGATTTTTAC
AGTGTAGTTTTGGAAAAACTCTTAGCCTACCAATTCTTCTAAGTGTTTTAAAATGTG
GGAGCCAGTACACATGAAGTTATAGAGTGTTTTAATGAGGCTTAAATATTTACCGTA
ACTATGAAATGCTACGCATATCATGCTGTTCAGGCTCCGTGGCCACGCAACTCggagtA
CACCCACCTGCTGTATTATCTCCAAACTCATGAACCACACCTTCACTGGTCCATTACT
TTCCTTTAAGAAAAATTTTTGGAGcaggttttctgacctccgtcggaaaaccGTTTACTTGGTTTTAAA
AATAGCTTGCACTAGCGATACGGAATATGGTTATTAGGTTTGTTAGGCATCATGTCG
TGTCTTACTATAGAAAAATAACGTAGTGTTCATTTTAGCCTGCCTGTATGTGTTAATT
TGTCCTTATTGCGCATTGTTCTTGTTAAGTCTTCTGTAAGGAGTTGCGGGTTTCAAAC
TGTCAGTCTGAGAGCAGAATTCGATATCTAGATCTCGAGGTAACCACGTGCGGACCC
AACGGCCGCaggaacccctagtgatggagttggccactccctctctgcgcgctcgctcgctcactgaggccgggcgaccaaagg
tcgcccgacgcccgggctttgcccgggcggcctcagtgagcgagcgagcgcgcagctgcctgcagg.

An illustrative AAV vector of the disclosure targeting SOD1 exon 2 is A05209. The elements of A05209 are set forth in Table 15. In some aspects a nucleic acid sequence encoding AAV vector A05209 comprises SEQ ID NO: 46.

TABLE 15
A05209 vector-scAAV-2x_mU7prom-SOD1z13/z14-mU7term_mU1prom-
SOD1z13/z14-mU1term; mouseloop with5′ ISD and eSL; Nucleotide sequences of plasmid
elements in order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z13/z14 | TCCCCACACCTTCACTGGTCCATTACTTTCCTTTAACC |
| | ACCTGCTGTATTATCTCCAAACTCATGAACATGG SEQ |
| | ID NO: 31 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z13/z14 | TCCCCACACCTTCACTGGTCCATTACTTTCCTTTAACC |
| | ACCTGCTGTATTATCTCCAAACTCATGAACATGG SEQ |
| | ID NO: 31 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A05209 Nucleotide Sequence (whole transgene from ITR to ITR):

(SEQ ID NO: 46)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccgaata
aggaactgtgctttgtgattcacatatcagtggaggggtgtggaaatggcaccttgatctcaccctcatcgaaagtggagttgatgtcctTcc
ctggctcgctacagacgcacttccgcaaggagtTCCCCACACCTTCACTGGTCCATTACTTTCCTTTAACCA
CCTGCTGTATTATCTCCAAACTCATGAACATGGAATTTTTGGAGcaggttttctgacctccgtcgg
aaaacccccaatttcactggtctacaatgaaagcaaaacagttctcttccccgctccccggtgtgtgagaggggctttgatccttctctggtt
tcctaggaaacgcgtatgtgTTGTTCCTCTTAGTGTTAATTCACACTAAAGACTGTGCATCCGACT
CCTACATTTATGAAAGTAAATGCCTGTTGTTAGAACAAAAAAGGCTACAGAACAAA
AAACAAAGCGAAATACCATCTGCTTTAGGTTCAGTGTGGTATTTTCCCGCTGACAGG
GAGGCGGGTTTTTGGGTACAGGAAACGAGTCACTATGGAGGCGGTACTATGTAGAT
GAGAATTCAGGTGCAAACTGGGAAAAGCAACTGCTTCCAAATATTTGTGATTTTTAC
AGTGTAGTTTTGGAAAAACTCTTAGCCTACCAATTCTTCTAAGTGTTTTAAAATGTG
GGAGCCAGTACACATGAAGTTATAGAGTGTTTTAATGAGGCTTAAATATTTACCGTA
ACTATGAAATGCTACGCATATCATGCTGTTCAGGCTCCGTGGCCACGCAACTCggagtT
CCCCACACCTTCACTGGTCCATTACTTTCCTTTAACCACCTGCTGTATTATCTCCAAA
CTCATGAACATGGAATTTTTGGAGcaggttttctgacctccgtcggaaaaccGTTTACTTGGTTTTAA
AAATAGCTTGCACTAGCGATACGGAATATGGTTATTAGGTTTGTTAGGCATCATGTC
GTGTCTTACTATAGAAAAATAACGTAGTGTTCATTTTAGCCTGCCTGTATGTGTTAAT
TTGTCCTTATTGCGCATTGTTCTTGTTAAGTCTTCTGTAAGGAGTTGCGGGTTTCAAA
CTGTCAGTCTGAGAGCAGAATTCGATATCTAGATCTCGAGGTAACCACGTGCGGACC
CAACGGCCGCaggaacccctagtgatggagttggccactccctctctgcgcgctcgctcgctcactgaggccgggcgaccaaag
gtcgcccgacgcccgggctttgcccgggcggcctcagtgagcgagcgagcgcgcagctgcctgcagg.

An illustrative AAV vector of the disclosure targeting SOD1 exon 2 is A05210. The elements of A05210 are set forth in 16. In some aspects a nucleic acid sequence encoding AAV vector A05210 comprises SEQ ID NO: 47.

TABLE 16
A05210 vector-scAAV-2x_mU7prom-SOD1z14/z13-mU7term_mU1prom-
SOD1z14/z13-mU1term; mouseloop with5′ ISD and eSL; Nucleotide sequences of plasmid
elements in order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z14/z13 | CCACCTGCTGTATTATCTCCAAACTCATGAACATGGT |
| | CCCCACACCTTCACTGGTCCATTACTTTCCTTTAA |
| | SEQ ID NO: 32 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z14/z13 | CCACCTGCTGTATTATCTCCAAACTCATGAACATGGT |
| | CCCCACACCTTCACTGGTCCATTACTTTCCTTTAA |
| | SEQ ID NO: 32 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A04054 Nucleotide Sequence (whole transgene from ITR to ITR):

(SEQ ID NO: 47)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccgaata
aggaactgtgctttgtgattcacatatcagtggaggggtgtggaaatggcaccttgatctcaccctcatcgaaagtggagttgatgtcctTcc
ctggctcgctacagacgcacttccgcaaggagtCCACCTGCTGTATTATCTCCAAACTCATGAACATGGTC
CCCACACCTTCACTGGTCCATTACTTTCCTTTAAAATTTTTGGAGcaggttttctgacctccgtcgg
aaaacccccaatttcactggtctacaatgaaagcaaaacagttctcttccccgctccccggtgtgtgagaggggctttgatccttctctggtt
tcctaggaaacgcgtatgtgTTGTTCCTCTTAGTGTTAATTCACACTAAAGACTGTGCATCCGACT
CCTACATTTATGAAAGTAAATGCCTGTTGTTAGAACAAAAAAGGCTACAGAACAAA
AAACAAAGCGAAATACCATCTGCTTTAGGTTCAGTGTGGTATTTTCCCGCTGACAGG
GAGGCGGGTTTTTGGGTACAGGAAACGAGTCACTATGGAGGCGGTACTATGTAGAT
GAGAATTCAGGTGCAAACTGGGAAAAGCAACTGCTTCCAAATATTTGTGATTTTTAC
AGTGTAGTTTTGGAAAAACTCTTAGCCTACCAATTCTTCTAAGTGTTTTAAAATGTG
GGAGCCAGTACACATGAAGTTATAGAGTGTTTTAATGAGGCTTAAATATTTACCGTA
ACTATGAAATGCTACGCATATCATGCTGTTCAGGCTCCGTGGCCACGCAACTCggagtC
CACCTGCTGTATTATCTCCAAACTCATGAACATGGTCCCCACACCTTCACTGGTCCAT
TACTTTCCTTTAAAATTTTTGGAGcaggttttctgacctccgtcggaaaaccGTTTACTTGGTTTTAAA
AATAGCTTGCACTAGCGATACGGAATATGGTTATTAGGTTTGTTAGGCATCATGTCG
TGTCTTACTATAGAAAAATAACGTAGTGTTCATTTTAGCCTGCCTGTATGTGTTAATT
TGTCCTTATTGCGCATTGTTCTTGTTAAGTCTTCTGTAAGGAGTTGCGGGTTTCAAAC
TGTCAGTCTGAGAGCAGAATTCGATATCTAGATCTCGAGGTAACCACGTGCGGACCC
AACGGCCGCaggaacccctagtgatggagttggccactccctctctgcgcgctcgctcgctcactgaggccgggcgaccaaagg
tcgcccgacgcccgggctttgcccgggcggcctcagtgagcgagcgagcgcgcagctgcctgcagg.

An illustrative AAV vector of the disclosure targeting murine SOD1 exon 2 is A05323. The elements of A05323 are set forth in 17. In some aspects a nucleic acid sequence encoding AAV vector A05323 comprises SEQ ID NO: 48.

TABLE 17
A05323 vector-scAAV-2x_mU7prom-SOD1z9/z3-mU7term_mU1prom-SOD1z9/z3-
mU1term; mouseloop with5′ ISD and eSL; Nucleotide sequences of plasmid elements in
order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z9/z3 | tgTGTATTgTCcCCAtACTgATGgACgTCCtgACAaCacaAC |
| | TGGTtCAccg SEQ ID NO: 35 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z9/z3 | tgTGTATTgTCcCCAtACTgATGgACgTCCtgACAaCacaAC |
| | TGGTtCAccg SEQ ID NO: 35 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A05323 Nucleotide Sequence (whole transgene from ITR to ITR):

(SEQ ID NO: 48)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccgaata
aggaactgtgctttgtgattcacatatcagtggaggggtgtggaaatggcaccttgatctcaccctcatcgaaagtggagttgatgtcctTcc
ctggctcgctacagacgcacttccgcaaggagttgTGTATTgTCcCCAtACTgATGgACgTCCtgACAaCacaACT
GGTtCAccgAATTTTTGGAGcaggttttctgacctccgtcggaaaacccccaatttcactggtctacaatgaaagcaaaacag
ttctcttccccgctccccggtgtgtgagaggggctttgatccttctctggtttcctaggaaacgcgtatgtgTTGTTCCTCTTAGTG
TTAATTCACACTAAAGACTGTGCATCCGACTCCTACATTTATGAAAGTAAATGCCTG
TTGTTAGAACAAAAAAGGCTACAGAACAAAAAACAAAGCGAAATACCATCTGCTTT
AGGTTCAGTGTGGTATTTTCCCGCTGACAGGGAGGCGGGTTTTTGGGTACAGGAAAC
GAGTCACTATGGAGGCGGTACTATGTAGATGAGAATTCAGGTGCAAACTGGGAAAA
GCAACTGCTTCCAAATATTTGTGATTTTTACAGTGTAGTTTTGGAAAAACTCTTAGCC
TACCAATTCTTCTAAGTGTTTTAAAATGTGGGAGCCAGTACACATGAAGTTATAGAG
TGTTTTAATGAGGCTTAAATATTTACCGTAACTATGAAATGCTACGCATATCATGCT
GTTCAGGCTCCGTGGCCACGCAACTCggagttgTGTATTgTCcCCAtACTgATGgACgTCCtg
ACAaCacaACTGGTtCAccgAATTTTTGGAGcaggttttctgacctccgtcggaaaaccGTTTACTTGGTT
TTAAAAATAGCTTGCACTAGCGATACGGAATATGGTTATTAGGTTTGTTAGGCATCA
TGTCGTGTCTTACTATAGAAAAATAACGTAGTGTTCATTTTAGCCTGCCTGTATGTGT
TAATTTGTCCTTATTGCGCATTGTTCTTGTTAAGTCTTCTGTAAGGAGTTGCGGGTTT
CAAACTGTCAGTCTGAGAGCAGAATTCGATATCTAGATCTCGAGGTAACCACGTGC
GGACCCAACGGCCGCaggaacccctagtgatggagttggccactccctctctgcgcgctcgctcgctcactgaggccgggc
gaccaaaggtcgcccgacgcccgggctttgcccgggcggcctcagtgagcgagcgagcgcgcagctgcctgcagg.

An illustrative AAV vector of the disclosure targeting SOD1 exon 2 is A05325. The elements of A05325 are set forth in 18. In some aspects a nucleic acid sequence encoding AAV vector A05325 comprises SEQ ID NO: 49.

TABLE 18
A05325 vector-scAAV-2x_mU7prom-SOD1z9/z3-mU7term_mU1prom-SOD1z9/23-
mU1term; mouseloop with 5′ ISD and eSL; Nucleotide sequences of plasmid elements in
order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z10/z2 | ACCTtgTGTATTgTCcCCAtACTgATGgACgACAaCacaAC |
| | TGGTtCAccgCTTgCCTTg SEQ ID NO: 36 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z10/z2 | ACCTtgTGTATTgTCcCCAtACTgATGgACgACAaCacaAC |
| | TGGTtCAccgCTTgCCTTg SEQ ID NO: 36 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A05325 Nucleotide Sequence (whole transgene from ITR to ITR):

(SEQ ID NO: 49)
Ctgcgcgctcgctcgctcactgaggccgcccgggcaaagcccgggcgtcgggcgacctttggtcgcccggcctcagtgagcgagcga
gcgcgcagagagggagtggggttCTTCGAAACACCGGTtaacaacataggagctgtgattggctgttttcagccaatcagca
ctgActcatttgcatagcctttacaagcggtcacaaactcaagaaacgagcggttttaatagtcttttagaatattgtttatcgaaccgaata
aggaactgtgctttgtgattcacatatcagtggaggggtgtggaaatggcaccttgatctcaccctcatcgaaagtggagttgatgtcctTcc
ctggctcgctacagacgcacttccgcaaggagtACCTtgTGTATTgTCcCCAtACTgATGgACgACAaCacaACT
GGTtCAccgCTTgCCTTgAATTTTTGGAGcaggttttctgacctccgtcggaaaacccccaatttcactggtctacaatg
aaagcaaaacagttctcttccccgctccccggtgtgtgagaggggctttgatccttctctggtttcctaggaaacgcgtatgtgTTGTT
CCTCTTAGTGTTAATTCACACTAAAGACTGTGCATCCGACTCCTACATTTATGAAAG
TAAATGCCTGTTGTTAGAACAAAAAAGGCTACAGAACAAAAAACAAAGCGAAATAC
CATCTGCTTTAGGTTCAGTGTGGTATTTTCCCGCTGACAGGGAGGCGGGTTTTTGGG
TACAGGAAACGAGTCACTATGGAGGCGGTACTATGTAGATGAGAATTCAGGTGCAA
ACTGGGAAAAGCAACTGCTTCCAAATATTTGTGATTTTTACAGTGTAGTTTTGGAAA
AACTCTTAGCCTACCAATTCTTCTAAGTGTTTTAAAATGTGGGAGCCAGTACACATG
AAGTTATAGAGTGTTTTAATGAGGCTTAAATATTTACCGTAACTATGAAATGCTACG
CATATCATGCTGTTCAGGCTCCGTGGCCACGCAACTCggagtACCTtgTGTATTgTCcCCA
tACTgATGgACgACAaCacaACTGGTtCAccgCTTgCCTTgAATTTTTGGAGcaggttttctgacctccg
tcggaaaaccGTTTACTTGGTTTTAAAAATAGCTTGCACTAGCGATACGGAATATGGTTAT
TAGGTTTGTTAGGCATCATGTCGTGTCTTACTATAGAAAAATAACGTAGTGTTCATTT
TAGCCTGCCTGTATGTGTTAATTTGTCCTTATTGCGCATTGTTCTTGTTAAGTCTTCTG
TAAGGAGTTGCGGGTTTCAAACTGTCAGTCTGAGAGCAGAATTCGATATCTAGATCT
CGAGGTAACCACGTGCGGACCCAACGGCCGCaggaacccctagtgatggagttggccactccctctctgcgc
gctcgctcgctcactgaggccgggcgaccaaaggtcgcccgacgcccgggctttgcccgggcggcctcagtgagcgagcgagcgcgc
agctgcctgcagg.

Provided herein is a vector targeting SOD1 comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 38-50. In some embodiments, the vector targeting SOD1 comprises the nucleic acid sequence set forth in SEQ ID NO: 38. In some embodiments, the vector targeting SOD1 comprises the nucleic acid sequence set forth in SEQ ID NO: 39. In some embodiments, the vector targeting SOD1 comprises the nucleic acid sequence set forth in SEQ ID NO: 40. In some embodiments, the vector targeting SOD1 comprises the nucleic acid sequence set forth in SEQ ID NO: 41. In some embodiments, the vector targeting SOD1 comprises the nucleic acid sequence set forth in SEQ ID NO: 42. In some embodiments, the vector targeting SOD1 comprises the nucleic acid sequence set forth in SEQ ID NO: 43. In some embodiments, the vector targeting SOD1 comprises the nucleic acid sequence set forth in SEQ ID NO: 44. In some embodiments, the vector targeting SOD1 comprises the nucleic acid sequence set forth in SEQ ID NO: 45. In some embodiments, the vector targeting SOD1 comprises the nucleic acid sequence set forth in SEQ ID NO: 46. In some embodiments, the vector targeting SOD1 comprises the nucleic acid sequence set forth in SEQ ID NO: 47. In some embodiments, the vector targeting SOD1 comprises the nucleic acid sequence set forth in SEQ ID NO: 48. In some embodiments, the vector targeting SOD1 comprises the nucleic acid sequence set forth in SEQ ID NO: 49. In some embodiments, the vector targeting SOD1 comprises the nucleic acid sequence set forth in SEQ ID NO: 50.

### Knockdown and Replacement Vectors

In some aspects, snRNA molecules of the disclosure target endogenous SOD1 wild-type (WT) and mutant variants. In some aspects, compositions of the disclosure further comprise a hardened nucleic acid sequence encoding a wild-type SOD1 protein. Accordingly, the mRNA encoding the endogenous toxic SOD1 protein is knocked down and replaced by an mRNA encoding healthy and fully functioning SOD1, which is encoded by a modified nucleic acid (“hardened”) so that the snRNA won't bind and target the mRNA encoding the replacement SOD1 copy. In some aspects, the nucleic acid sequence encoding the replacement SOD1 is a “hardened” SOD1 nucleic acid sequence. In some aspects, a hardened nucleic acid sequence is a sequence that, while encoding wild-type SOD1, is codon optimized such that the snRNA compositions of the disclosure cannot bind the hardened SOD1 nucleic acid sequence. In some aspects, the hardened SOD1 nucleic acid sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 131. In some aspects, the hardened SOD1 nucleic acid sequence comprises, consists essentially of, or consists of a nucleic acid sequence of SEQ ID NO: 131, or a sequence having 1, 2, 3, 4, 5, 6, 7, 8, 9 or 10 substitutions, insertions or deletions relative thereto.

In some aspects, the disclosure provides a recombinant AAV (rAAV) comprising a nucleic acid sequence encoding a first small nuclear RNA (snRNA) molecule, wherein the first snRNA comprises a targeting sequence that binds a target SOD1 RNA sequence and a nucleic acid sequence encoding a hardened SOD1 nucleic acid sequence encoding a SOD1 polypeptide. In some aspects, the rAAV can comprise a nucleic acid sequence encoding a second snRNA molecule comprising a targeting sequence that binds a target SOD1 RNA sequence. In some aspects, the rAAV can comprise a nucleic acid sequence encoding a third snRNA molecule comprising a targeting sequence that binds a target SOD1 RNA sequence and a fourth snRNA molecule comprising a targeting sequence that binds a target SOD1 RNA sequence. In some embodiments, the first, second, third and/or fourth snRNA bind different sequences within the target SOD1 RNA sequence. In some embodiments, the first, second, third and/or fourth snRNA bind the same sequence within the target SOD1 RNA sequence.

In some aspects, the first snRNA is operably linked to a first promoter and the second snRNA is operably linked to a second promoter. In some embodiments, the first promoter and the second promoter are the same promoter. In some embodiments, the first promoter and the second promoter are different promoters. In some aspects, the promoter operably linked to the snRNA molecule is a U1, U2, U4 U5, U6, or U7 promoter or any other suitable promoter disclosed herein or known in the art. In some aspects, expression of the hardened SOD1 nucleic acid sequence is directed by a promoter. Any suitable promoter may be uses to direct expression of the hardened SOD1 nucleic acid sequence. In some aspects, the promoter is a Central nervous system specific promoter. Nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-0), the synapsin (Syn), the methyl-CpG binding protein 2 (MeCP2), Ca2+/calmodulin-dependent protein kinase II (CaMKII), metabotropic glutamate receptor 2 (mGluR2), homeobox HB9, NFL, NFH, nj32, PPE, Enk and EAAT2 promoters. A non-limiting example of a tissue-specific expression elements for astrocytes include the glial fibrillary acidic protein (GFAP) and EAAT2 promoters. A non-limiting example of a tissue-specific expression element for oligodendrocytes includes the myelin basic protein (MBP) promoter.

In one embodiment, the encoded payload construct comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B 1-CBX3).

In aspects, the promoter is a pJET promoter. In some aspects, the pJet promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 132. In some aspects, the pJet promoter comprises, consists essentially of, or consists of the nucleic acid sequence of SEQ ID NO: 132, or a sequence having 1, 2, 3, 4, 5, 6, 7, 8, 9 or 10 substitutions, insertions or deletions relative thereto. In some aspects, the vector comprises: a nucleic acid sequence encoding a first inverted terminal repeat (ITR) sequence; a nucleic acid sequence encoding a first promoter sequence; a nucleic acid sequence encoding a first snRNA sequence wherein the first snRNA comprises a targeting sequence that binds a target SOD1 RNA sequence; a nucleic acid sequence encoding a second promoter sequence; a nucleic acid sequence encoding a second snRNA sequence wherein the first snRNA comprises a targeting sequence that binds a target SOD1 RNA sequence; a nucleic acid sequence encoding a third promoter sequence; a nucleic acid sequence encoding a hardened SOD1 nucleic acid sequence encoding a SOD1 polypeptide; a nucleic acid sequence encoding a polyA sequence; a nucleic acid sequence encoding a second ITR sequence.

In some aspects, vectors of the disclosure are configured such that hardened SOD1 is expressed from the sense strand of the vector. In some aspects, vectors of the disclosure are configured such that hardened SOD1 is expressed from the antisense strand of the vector.

An illustrative AAV vector of the disclosure targeting SOD1 exon 2 and expressing a SOD1 hardened/WT replacement is A05433. The elements of A05433 are set forth in 19. In some aspects a nucleic acid sequence encoding AAV vector A05433 comprises SEQ ID NO: 50.

TABLE 19
A05433 vector-scAAV-2x_mU7prom-SOD1z3/z10-mU7term_mU1prom-SOD1z3/z10-
mU1term; MSloop with 5′ ISD and eSL; pJet_SOD1 (codon opt)_SV40 (AS strand); Nucleotide
sequences of plasmid elements in order 5′ to 3′

| Plasmid Element | Nucleotide Sequences |
|---|---|
| 5′ITR | Ctgcgcgctcgctcgctcactgaggccgcccgggcaaagcccgggcgtcgggcga |
| | cctttggtcgcccggcctcagtgagcgagcgagcgcgcagagagggagtggggtt |
| | SEQ ID NO: 125 |
| Mouse U7 Promoter | taacaacataggagctgtgattggctgttttcagccaatcagcactgActcatttgcatag |
| | cctttacaagcggtcacaaactcaagaaacgagcggttttaatagtcttttagaatattgttt |
| | atcgaaccgaataaggaactgtgctttgtgattcacatatcagtggaggggtgtggaaat |
| | ggcaccttgatctcaccctcatcgaaagtggagttgatgtcctTccctggctcgctacag |
| | acgcacttccgc SEQ ID NO: 101 |
| extra nt | aa |
| ISD | ggagt |
| Fusion spacer z3/z10 | TTCCCCACACCTTCACTGGTCCATTAACCTGCTGTAT |
| | TATCTCCAAACTCATGAAC SEQ ID NO: 19 |
| SMOPT | AATTTTTGGAGca SEQ ID NO: 127 |
| Stem loop | Ggttttctgacctccgtcggaaaacc SEQ ID NO: 66 |
| Mouse U7 terminator | Cccaatttcactggtctacaatgaaagcaaaacagttctcttccccgctccccggtgtgt |
| | gagaggggctttgatccttctctggtttcctaggaaacgcgtatgtg SEQ ID NO: |
| | 113 |
| Mouse U1 Promoter | TTGTTCCTCTTAGTGTTAATTCACACTAAAGACTGTG |
| | CATCCGACTCCTACATTTATGAAAGTAAATGCCTGTT |
| | GTTAGAACAAAAAAGGCTACAGAACAAAAAACAAA |
| | GCGAAATACCATCTGCTTTAGGTTCAGTGTGGTATTT |
| | TCCCGCTGACAGGGAGGCGGGTTTTTGGGTACAGGA |
| | AACGAGTCACTATGGAGGCGGTACTATGTAGATGAG |
| | AATTCAGGTGCAAACTGGGAAAAGCAACTGCTTCCA |
| | AATATTTGTGATTTTTACAGTGTAGTTTTGGAAAAAC |
| | TCTTAGCCTACCAATTCTTCTAAGTGTTTTAAAATGT |
| | GGGAGCCAGTACACATGAAGTTATAGAGTGTTTTAA |
| | TGAGGCTTAAATATTTACCGTAACTATGAAATGCTAC |
| | GCATATCATGCTGTTCAGGCTCCGTGGCCACGCAACT |
| | C SEQ ID NO: 99 |
| ISD | ggagt |
| Fusion spacer z3/z10 | TTCCCCACACCTTCACTGGTCCATTAACCTGCTGTAT |
| | TATCTCCAAACTCATGAAC SEQ ID NO: 19 |
| SMOPT | AATTTTTGGAGca SEQ ID NO: 127 |
| Stem loop | Ggttttctgacctccgtcggaaaacc SEQ ID NO: 66 |
| Mouse U1 terminator | GTTTACTTGGTTTTAAAAATAGCTTGCACTAGCGATA |
| | CGGAATATGGTTATTAGGTTTGTTAGGCATCATGTCG |
| | TGTCTTACTATAGAAAAATAACGTAGTGTTCATTTTA |
| | GCCTGCCTGTATGTGTTAATTTGTCCTTATTGCGCATT |
| | GTTCTTGTTAAGTCTTCTGTAAGGAGTTGCGGGTTTC |
| | AAACTGTCAGTCTGAGAGCA SEQ ID NO: 111 |
| SV40 PolyA (Reverse | Taagatacattgatgagtttggacaaaccacaactagaatgcagtgaaaaaaatgcttta |
| Orientation) | tttgtgaaatttgtgatgctattgctttatttgtaaccattataagctgcaataaacaagtt |
| | SEQ ID NO: 129 |
| HA Tag (Reverse | GGCGTAGTCGGGCACATCGTAGGGGTA SEQ ID NO: |
| Orientation) | 130 |
| Linker | GTCCTC |
| Codon Optimized SOD1 | CTGGGCGATTCCAATCACGCCGCAGGCCAGTCTGCT |
| CDS (Reverse Orientation) | GCCGGCGTTGCCGGTCTTGGTGCTTTCCTCGTTGCCG |
| | CCTTTGCCCAGGTCGTCGGCCTTCTCGTGCACCACCA |
| | GGGTTCTGCCGATGATGCAGTGGTCGCCGCTCAGGCT |
| | GATCACGCTGTCCTCGATGGACACATCGGCGACACC |
| | ATCCTTATCAGCTGTCACATTTCCCAGGTCGCCCACG |
| | TGCCGTTCCTCGTCCTTAGGTCCGCCGTGCTTTCTAG |
| | ACAGGGGGTTGAAGTGAGGGCCGGCGCTTGTACATC |
| | CGGCGGTGTTGTCGCCGAATTCATGAACGTGAAAGC |
| | CGTGGAGGCCCTCGGTGAGGCCCTTGATAGATCCCC |
| | AGACTTTGACGGGGCCGTTGGATTCCTTCTGCTCGAA |
| | GTTGATGATGCCCTGCACAGGGCCATCGCCCTTCAGC |
| | ACGCACACGGCTTTGGTAGCCAT SEQ ID NO: 131 |
| Kozak Sequence | GGTggcggc |
| pJET Promoter (Reverse | Tgtcaagtgacgatcacagggatccacaaacaagaaccgcgacccaaatcccggctg |
| Orientation) | cgacggaactagctgtgccacacccggcgcgtccttatataatcatcggcgttcaccgc |
| | ccattctccgcccagccataaaaggcaactttcggaacggcgcacgctgattggctccg |
| | ccctaactccgcccgaattc SEQ ID NO: 132 |
| 3′ITR | Aggaacccctagtgatggagttggccactccctctctgcgcgctcgctcgctcactgag |
| | gccgggcgaccaaaggtcgcccgacgcccgggctttgcccgggcggcctcagtgag |
| | cgagcgagcgcgcagctgcctgcagg SEQ ID NO: 126 |

A05433 Nucleotide Sequence (whole transgene from ITR to ITR):

(SEQ ID NO: 50)
ctgcgcgctcgctcgctcactgaggccgcccgggcaaagcccgggcgtcgggcgacctttggtcgcccggcctcagtgagcgagcgag
cgcgcagagagggagtggggttCTTCGAAACACCGGTtaacaacataggagctgtgattggctgttttcagccaatcagcac
tgActcatttgcatagcctttacaagcggtcacaaactcaagaaacgagcggttttaatagtcttttagaatattgtttatcgaaccgaataa
ggaactgtgctttgtgattcacatatcagtggaggggtgtggaaatggcaccttgatctcaccctcatcgaaagtggagttgatgtcctTccc
tggctcgctacagacgcacttccgcaaggagtTTCCCCACACCTTCACTGGTCCATTAACCTGCTGTATTA
TCTCCAAACTCATGAACAATTTTTGGAGcaggttttctgacctccgtcggaaaacccccaatttcactggtctacaa
tgaaagcaaaacagttctcttccccgctccccggtgtgtgagaggggctttgatccttctctggtttcctaggaaacgcgtatgtgTTGT
TCCTCTTAGTGTTAATTCACACTAAAGACTGTGCATCCGACTCCTACATTTATGAAA
GTAAATGCCTGTTGTTAGAACAAAAAAGGCTACAGAACAAAAAACAAAGCGAAAT
ACCATCTGCTTTAGGTTCAGTGTGGTATTTTCCCGCTGACAGGGAGGCGGGTTTTTG
GGTACAGGAAACGAGTCACTATGGAGGCGGTACTATGTAGATGAGAATTCAGGTGC
AAACTGGGAAAAGCAACTGCTTCCAAATATTTGTGATTTTTACAGTGTAGTTTTGGA
AAAACTCTTAGCCTACCAATTCTTCTAAGTGTTTTAAAATGTGGGAGCCAGTACACA
TGAAGTTATAGAGTGTTTTAATGAGGCTTAAATATTTACCGTAACTATGAAATGCTA
CGCATATCATGCTGTTCAGGCTCCGTGGCCACGCAACTCggagtTTCCCCACACCTTCA
CTGGTCCATTAACCTGCTGTATTATCTCCAAACTCATGAACAATTTTTGGAGcaggttttct
gacctccgtcggaaaaccGTTTACTTGGTTTTAAAAATAGCTTGCACTAGCGATACGGAATAT
GGTTATTAGGTTTGTTAGGCATCATGTCGTGTCTTACTATAGAAAAATAACGTAGTG
TTCATTTTAGCCTGCCTGTATGTGTTAATTTGTCCTTATTGCGCATTGTTCTTGTTAAG
TCTTCTGTAAGGAGTTGCGGGTTTCAAACTGTCAGTCTGAGAGCAGAATTCGATATC
TAGATCtaagatacattgatgagtttggacaaaccacaactagaatgcagtgaaaaaaatgctttatttgtgaaatttgtgatgctattgctt
tatttgtaaccattataagctgcaataaacaagttGTTAAGGCGGCCGCGTTAAGTCAGGCGTAGTCGGGC
ACATCGTAGGGGTAGTCCTCCTGGGCGATTCCAATCACGCCGCAGGCCAGTCTGCTG
CCGGCGTTGCCGGTCTTGGTGCTTTCCTCGTTGCCGCCTTTGCCCAGGTCGTCGGCCT
TCTCGTGCACCACCAGGGTTCTGCCGATGATGCAGTGGTCGCCGCTCAGGCTGATCA
CGCTGTCCTCGATGGACACATCGGCGACACCATCCTTATCAGCTGTCACATTTCCCA
GGTCGCCCACGTGCCGTTCCTCGTCCTTAGGTCCGCCGTGCTTTCTAGACAGGGGGT
TGAAGTGAGGGCCGGCGCTTGTACATCCGGCGGTGTTGTCGCCGAATTCATGAACGT
GAAAGCCGTGGAGGCCCTCGGTGAGGCCCTTGATAGATCCCCAGACTTTGACGGGG
CCGTTGGATTCCTTCTGCTCGAAGTTGATGATGCCCTGCACAGGGCCATCGCCCTTC
AGCACGCACACGGCTTTGGTAGCcatGGTggcggcGCTAGCTAGTTAAGCGTGAtgtcaagtg
acgatcacagggatccacaaacaagaaccgcgacccaaatcccggctgcgacggaactagctgtgccacacccggcgcgtccttatata
atcatcggcgttcaccgcccattctccgcccagccataaaaggcaactttcggaacggcgcacgctgattggctccgccctaactccgccc
gaattcAGTTAAGTCGAGGTAACCACGTGCGGACCCAACGGCCGCaggaacccctagtgatggagtt
ggccactccctctctgcgcgctcgctcgctcactgaggccgggcgaccaaaggtcgcccgacgcccgggctttgcccgggggcctca
gtgagcgagcgagcgcgcagctgcctgcagg.

### Nucleic Acids

An NOI (nucleotide sequence of interest) includes, without limitation, any nucleotide sequence or transgene capable of being delivered by a vector. NOIs can be synthetic, derived from naturally occurring DNA or RNA, codon optimized, recombinant RNA/DNA, cDNA, partial genomic DNA, and/or combinations thereof. The NOI can be a coding region or partial coding region but need not be a coding region. An NOI can be RNA/DNA in a sense or anti-sense orientation. An NOI can be an snRNA. NOIs are also referred herein, without limitation, as transgenes, heterologous sequences, genes, therapeutic genes. An NOI may also encode an RNA (ribonucleoprotein complex) a POI (protein of interest, e.g. SOD1), a partial POI, a mutated version or variant of a POI. A POI may be analogous to or correspond to a wild-type protein. A POI may also be a fusion protein or ribonucleoprotein complex such as an snRNP. In some aspects RNA sequences disclosed herein may be represented as DNA sequences and it is within the ability of the skilled artisan to derive the sequence of an RNA sequence from a DNA sequence. For example, spacer sequences of the disclosure can represent uracil bases as either a U or T. The skilled artisan would readily understand that an RNA sequence can interchangeably use a T or U to indicate a uracil.

### Codon Optimization

In some embodiments, NOIs or transgenes such as nucleic acid sequences of the disclosure are codon optimized nucleic acid sequences.

Codon-optimization is a technique well known in the art. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences can be generated. In some embodiments, such a sequence is optimized for expression in a host or target cell, such as a host cell used to express the snRNA or a cell in which the disclosed methods are practiced (such as in a mammalian cell, e.g., a human cell). Codon preferences and codon usage tables for a particular species can be used to engineer isolated nucleic acid molecules encoding an snRNA that takes advantage of the codon usage preferences of that particular species. In some embodiments, an isolated nucleic acid molecule (which can be part of a vector) includes at least one coding sequence that is codon optimized for expression in a eukaryotic cell, or at least one coding sequence codon optimized for expression in a human cell. In one embodiment, such a codon optimized coding sequence has at least 80%, at least 85%, at least 90%, at least 92%, at least 95%, at least 96%, at least 97%, at least 98%, at least 99%, or 100% sequence identity to its corresponding wildtype or originating sequence. In another embodiment, a eukaryotic cell codon optimized nucleic acid sequence encodes snRNA having at least 85%, at least 90%, at least 92%, at least 95%, at least 96%, at least 97%, at least 98%, at least 99%, or 100% sequence identity to its corresponding wildtype or originating sequence. In another embodiment, a variety of clones containing functionally equivalent nucleic acids may be routinely generated, such as nucleic acids which differ in sequence but which encode the same sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., NY).

In some embodiments, the codon optimized sequence exhibits at least 5%, at least 10%, at least 20%, at least 30%, at least 50%, at least 75%, at least 100%, at least 200%, at least 300%, at least 500%, or at least 1000% increased transcription or translation in a human subject relative to a wild-type or non-codon optimized nucleic acid sequence.

In some aspects a codon optimized nucleic acid sequence exhibits increased stability. In some aspects a codon optimized nucleic acid sequence exhibits increased stability through increased resistance to hydrolysis. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased stability relative to a wild-type or non-codon optimized nucleic acid sequence. In some embodiments, the codon optimized sequence exhibits at least 5%, at least 10%, at least 20%, at least 30%, at least 50%, at least 75%, at least 100%, at least 200%, at least 300%, at least 500%, or at least 1000% increased resistance to hydrolysis in a human subject relative to a wild-type or non-codon optimized nucleic acid sequence.

In some aspects a codon optimized nucleic acid sequence can comprise no donor splice sites. In some aspects a codon optimized nucleic acid sequence can comprise no more than about one, or about two, or about three, or about four, or about five, or about six, or about seven, or about eight, or about nine, or about ten donor splice sites. In some aspects a codon optimized nucleic acid sequence comprises at least one, or at least two, or at least three, or at least four, or at least five, or at least six, or at least seven, or at least eight, or at least nine, or at least ten fewer donor splice sites as compared to a non-codon optimized nucleic acid sequence.

Without wishing to be bound by theory, the removal of donor splice sites in the codon optimized nucleic acid sequence can unexpectedly and unpredictably increase expression of protein of interest in vivo, as cryptic splicing is prevented. Moreover, cryptic splicing may vary between different subjects, meaning that the expression level of a protein comprising donor splice sites may unpredictably vary between different subjects. Such unpredictability is unacceptable in the context of human therapy. Accordingly, the codon optimized nucleic acid sequences which lacks donor splice sites, unexpectedly and surprisingly allows for increased expression of the protein in human subjects and regularizes expression of the protein across different human subjects.

In some aspects a codon optimized nucleic acid sequence can have a GC content that differs from the GC content of the non-codon optimized nucleic acid sequence. In some aspects the GC content of a codon optimized nucleic acid sequence is more evenly distributed across the entire nucleic acid sequence, as compared to the non-codon optimized nucleic acid sequence.

Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects a codon optimized nucleic acid sequence can have fewer repressive microRNA target binding sites as compared to the non-codon optimized nucleic acid sequence. In some aspects, a codon optimized nucleic acid sequence can have at least one, or at least two, or at least three, or at least four, or at least five, or at least six, or at least seven, or at least eight, or at least nine, or at least ten, or at least ten fewer repressive microRNA target binding sites as compared to the non-codon optimized nucleic acid sequence.

Without wishing to be bound by theory, by having fewer repressive microRNA target binding sites, the codon optimized nucleic acid sequence unexpectedly exhibits increased expression in a human subject.

It should be understood, although not always explicitly stated that the sequences provided herein can be used to provide the expression product as well as substantially identical sequences that encode an RNA or express and produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25 °C to about 37 °C; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40 °C to about 50 °C; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55 °C to about 68 °C; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. “Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention. To determine sequence identity, sequences can be aligned using the methods and computer programs that are known in the art, including BLAST, available over the world wide web at ncbi.nlm.nih.gov/BLAST.

As used herein, the terms “about” and “approximately” refer to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. Also, where ranges and/or subranges of values are provided, the ranges and/or subranges can include the endpoints of the ranges and/or subranges.

The term “operably linked” and “operably joined” or related terms as used herein refers to the juxtaposition of components. The components can be linked together covalently. For example, two nucleic acids can be linked together via a phosphodiester linkage. Alternatively a first component that confers a function on a second component without being directly physically linked can be considered to be operably linked.

### Cells

Also provided herein are cells comprising the RNA targeting systems and expression constructs described herein, compositions comprising same, and methods of making same. In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell. In some embodiments, the cell is a human cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is a muscle cell. In some embodiments, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell. In one embodiment, a cell or cells of a patient treated with compositions disclosed herein include, without limitation, skeletal muscle (developing and mature muscle fibers and satellite cells), neuromuscular junction, cardiomyocytes, smooth muscle cells, peripheral nervous system (neurons), peripheral motor neurons, and/or sensory neurons.

In some embodiments of the compositions and methods of the disclosure, a somatic cell of the disclosure is a neuronal cell. In one embodiment, a cell or cells of a patient treated with compositions disclosed herein include, without limitation, central nervous system (neurons), peripheral nervous system (neurons), peripheral motor neurons, sensory neuron, cortical or GABAergic inhibitory interneurons. In one embodiment, a neuronal cell is a glial cell.

In some embodiments of the compositions and methods of the disclosure, a somatic cell of the disclosure is a fibroblast or an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the disclosure, a somatic cell is an ocular cell. An ocular cell includes, without limitation, corneal epithelial cells, keratyocytes, retinal pigment epithelial (RPE) cells, lens epithelial cells, iris pigment epithelial cells, conjunctival fibroblasts, non-pigmented ciliary epithelial cells, trabecular meshwork cells, ocular choroid fibroblasts, conjunctival epithelial cells. In some embodiments, an ocular cell is a retinal cell or a corneal cell. In one embodiment, a retinal cell is a photoreceptor cell or a retinal pigment epithelial cell. In another embodiment, a retinal cell is a ganglion cell, an amacrine cell, a bipolar cell, a horizontal cell, a Müller glial cell, a rod cell, or a cone cell. In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Methods of Use

The disclosure provides a method of encoding an RNA or expressing an NOI (e.g., such as a hardened SOD1 mRNA) in a cell using the snRNA systems disclosed herein. In one embodiment, the disclosure provides a method of modifying an RNA encoding SOD1 or the activity of a SOD1 protein encoded by an RNA molecule comprising contacting the composition of the disclosure and the target RNA molecule under conditions suitable for binding to the RNA molecule.

The disclosure provides a method of modifying the level of expression of a SOD1 RNA molecule of the disclosure, or a SOD1 protein encoded by the SOD1 RNA molecule comprising contacting the composition of the disclosure and a cell comprising the SOD1 RNA molecule under conditions suitable for binding to the SOD1 RNA molecule. In some embodiments, the cell is in vivo, in vitro, ex vivo or in situ. In some embodiments, the composition of the disclosure comprises a vector comprising or encoding snRNA sequences. In some embodiments, the vector is an AAV.

The disclosure provides a method of modifying the level of expression of a SOD1 RNA molecule of the disclosure, or a protein encoded by the SOD1 RNA molecule comprising contacting a composition of the disclosure and the RNA molecule under conditions suitable for knocking down, blocking, splicing, multi-targeting, restore frame, or editing the SOD1 RNA. In some embodiments, the composition of the disclosure comprises a vector comprising snRNA sequences. In some embodiments, the vector is an AAV.

The disclosure provides a method of modifying a SOD1 RNA or an activity of a SOD1 protein encoded by the SOD1 RNA molecule comprising contacting a composition and a cell comprising the SOD1 RNA molecule under conditions suitable knocking down, blocking, splicing, multi-targeting, restore frame, or editing the SOD1 RNA. In some embodiments, the cell is in vivo, in vitro, ex vivo or in situ. In some embodiments, the composition comprises a vector comprising the snRNA sequences disclosed herein. In some embodiments, the vector is an AAV.

The disclosure provides a method of treating a disease or disorder comprising administering to a subject a therapeutically effective amount of an snRNA composition of the disclosure. In some embodiments, the disease comprises ALS.

The disclosure provides a method of treating a disease in a patient in need of such treatment comprising administering to the patient a therapeutically effective amount of an snRNA composition of the disclosure, wherein the composition comprises a vector comprising snRNA sequences disclosed herein, wherein the composition decreases a level of expression of a targeted RNA such as SOD1, and toxic mutations thereof including but not limited to as A4V, H46R, and G93A, (compared to the level of expression of a targeted RNA treated with a non-targeting (NT) control or compared to no treatment). In some embodiments, the levels of a SOD1 protein encoded by the targeted SOD1 RNA is reduced. In another embodiment, the level of decrease is 1-fold or greater. In another embodiment, the level of decrease is 2-fold, 3-fold, 4-fold, 5-fold, 6-fold, 7-fold, 8-fold, 9-fold or 10-fold. In another embodiment, the level of decrease is 10-fold or greater. In another embodiment, the level of decrease is between 10-fold and 20-fold. In another embodiment, the level of decrease is 11-fold, 12-fold, 13-fold, 14-fold, 15-fold, 16-fold, 17-fold, 18-fold, 19-fold, or 20-fold. In another embodiment, the gene therapy compositions disclosed herein when administered to a patient lead to 20%-100% decrease in expression of the RNA. In one embodiment, the % decrease and is any of 20-99%, 25%-99%, 50%-99%, 80%-99%, 90%-99%, 95%-99%. In one embodiment, the % decrease is 90%, 91%, 92%, 93%, 94%, 95%, 96%, 97%, 98%, or 99%. In another embodiment the gene therapy, sequences disclosed herein, promotes a decreases level of expression of the RNA transcript, decreasing protein expression and function. In another embodiment, % down-regulation is 1.5-fold or higher of the targeted RNA. In some embodiments, the targeted RNA is SOD1. In some embodiments, the targeted RNA is SOD1 exon 2. In some embodiments, the targeted RNA is SOD1 exon 3.

In some embodiments, methods of the disclosure further comprise replacing or restoring SOD1 expression wherein snRNA of the disclosure target and reduce expression of mutant SOD1 variants while also providing a nucleic acid sequence encoding a wild-type or healthy SOD1. In some embodiments, the nucleic acid sequence encoding the replacement SOD1 is a “hardened” SOD1 nucleic acid sequence. In some embodiments, the hardened nucleic acid sequence is a sequence that, while encoding wild-type SOD1, is codon optimized such that the snRNA compositions of the disclosure cannot bind the hardened SOD1 nucleic acid sequence.

The disclosure further provides a method of treating a disease or disorder in a subject comprising administering an RNA-targeting nucleic acid molecule (i.e. an snRNA of the disclosure) or an AAV vector comprising or encoding an snRNA of the disclosure.

In some aspects, the disease or disorder is Amyotrophic lateral sclerosis (ALS) and/or ALS/FTD.

In some aspects the RNA-targeting nucleic acid molecule or AAV vector targets an RNA sequence encoding SOD1. In some aspects the RNA sequence encoding SOD1 comprises an intronic or exonic sequence. In some aspects the exonic sequence comprises exon 2 or exon 3 or a flanking region thereof of SOD1.

In some embodiments of the methods of the disclosure, a subject of the disclosure has been diagnosed with a disease to be treated. In some embodiments, the subject of the disclosure presents at least one sign or symptom of a disorder or disease to be treated. In some embodiments, the subject of the disclosure presents at least one sign or symptom of a disease.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure a subject of the disclosure is a mammal. In some embodiments, a subject of the disclosure is a non-human mammal.

In some embodiments of the methods of the disclosure a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure a therapeutically effective amount eliminates the disease or disorder (e.g., ALS).

In some embodiments of the methods of the disclosure, a therapeutically effective amount prevents an onset of a disease or disorder. In some embodiments, a therapeutically effective amount delays the onset of a disease or disorder.

In some embodiments, a therapeutically effective amount improves a prognosis for the subject.

In some embodiments of the methods of the disclosure, a composition of the disclosure is administered to the subject via intracerebral administration. In some embodiments of the methods of the disclosure, a composition of the disclosure is administered to the subject by intracerebroventricular injection. In some embodiments, the composition of the disclosure is administered to the subject by an intrastriatal route. In some embodiments, the composition of the disclosure is administered to the subject by a stereotaxic injection or an infusion. In some embodiments, the composition is administered intravenously. In some embodiments, the composition is administered to the brain. In some embodiments of the methods of the disclosure a composition of the disclosure is administered to the subject locally.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intraparenchymal, intrastriatal, subcutaneous, intradermal, intraperitoneal, intratumoral, intravenous, intraocular, and/or parenteral administration. In certain embodiments, the compositions of the present disclosure are formulated for intracerebral or intrastriatal administration.

### EXAMPLES

The examples described herein are intended for illustration only and are not intended to limit the inventions claimed.

### Example 1: SOD1 Exon 2 Exclusion via SOD1 Targeting snRNA

### Material and Methods

### Material and Methods

Splicing assays to evaluate SOD1 exon skipping: 50-500 ng of RNA was used for cDNA synthesis with qScript Ultra SuperMix (QuantaBio) following manufacturer's recommendations. PCR was performed on cDNA with GoTaq Green Master Mix (Promega). PCR products were run on 4200 TapeStation (Agilent) with D1000 ScreenTape (Agilent).

qRT-PCR: Multiplexed TaqMan qRT-PCR was performed with Ultraplex 1-Step Toughmix (QuantaBio) and commercially available probe targeting hSOD1 with custom hGAPDH probe. Each sample was plated in duplicates and relative hSOD1 mRNA levels were calculated with the delta-delta Ct method.

Jess-protein expression: SOD1 protein analysis was performed on a Jess system (ProteinSimple) according to the manufacturer's instructions. Relative SOD1 protein levels were calculated by dividing area under the curve (AUC) of the SOD1 peak by AUC of the GAPDH peak, then normalizing to non-targeting negative control condition.

### Results

In Vitro Evaluation of U7 snRNAs Targeting SOD1 Exon 2

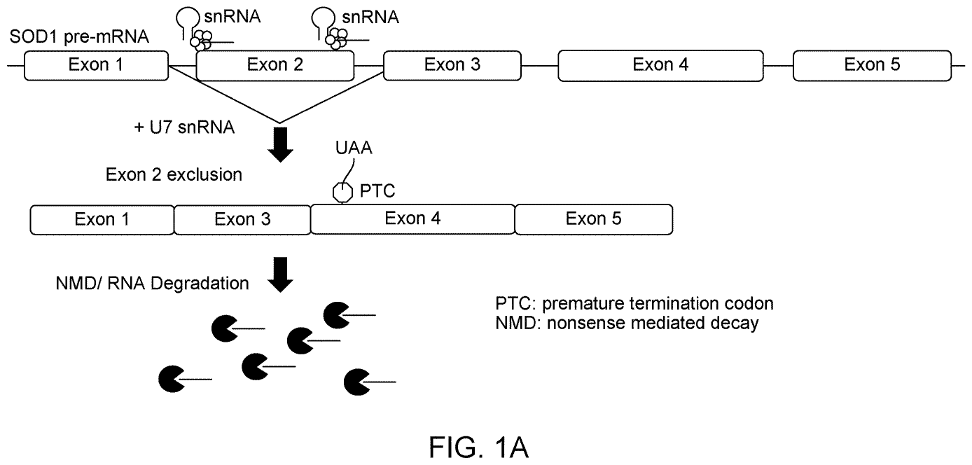

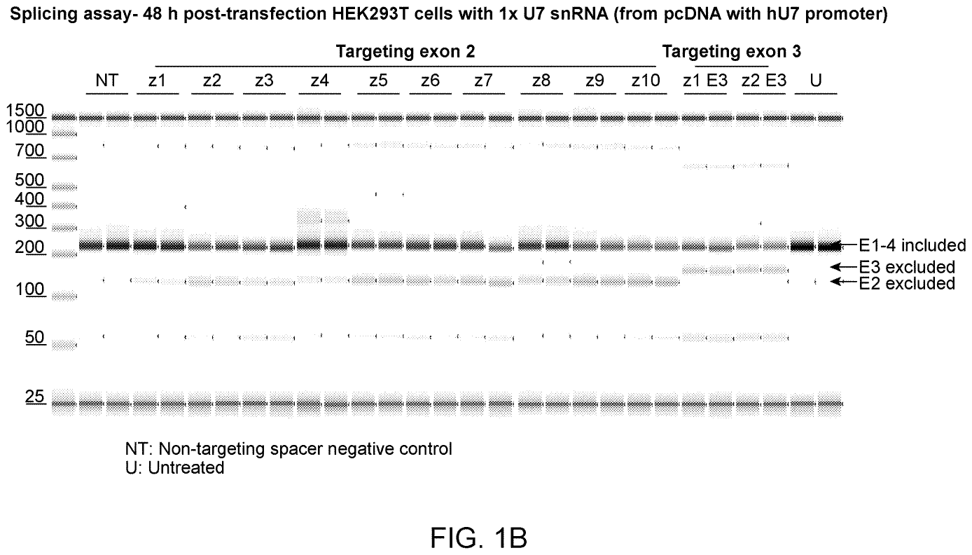

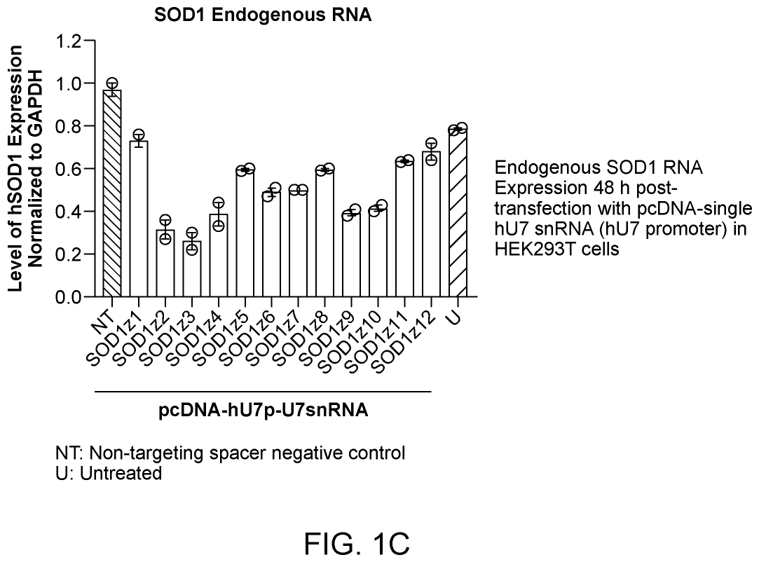

U7 snRNAs were engineered to bind splicing regulatory sequences within SOD1 pre-mRNA to promote skipping of a constitutive exon (exon 2 or 3) which will result in a frameshift and a premature termination codon (PTC) and nonsense mediated decay (NMD) with SOD1 RNA degradation (FIG. 1A).

FIG. 1A shows the mechanism of action for SOD1 knockdown (mutant toxic and WT) using engineered U7 snRNAs. U7 snRNAs were engineered to bind splicing regulatory sequences (acceptor and donor sites or splicing enhancer sequences) to promote skipping of a constitutive exon (SOD1 exon 2 or 3) which will lead to a frameshift and generation of a premature stop codon (PTC) to promote RNA degradation by the nonsense mediated decay (NMD) quality control system.

FIG. 1B shows a tapestation image of the RT-PCR products after U7 snRNA treatments using single U7 spacers, showing the SOD1 exon 1 to 4 PCR product (non-skipped band, top), and the skipped products (skipping of exon 3, middle band; or skipping of exon 2, bottom band). NT (non-targeting U7) was used as a negative control. FIG. 1C shows the qRT-PCR results for SOD1 RNA expression post-treatment with multiple U7 snRNAs with single spacers. Levels of human endogenous SOD1 RNA expression in HEK293T cells were normalized to GAPDH reference gene and the non-targeting spacer.

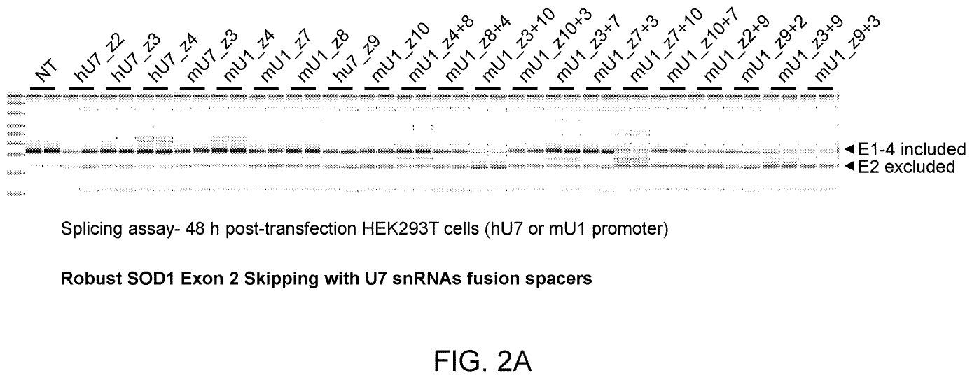

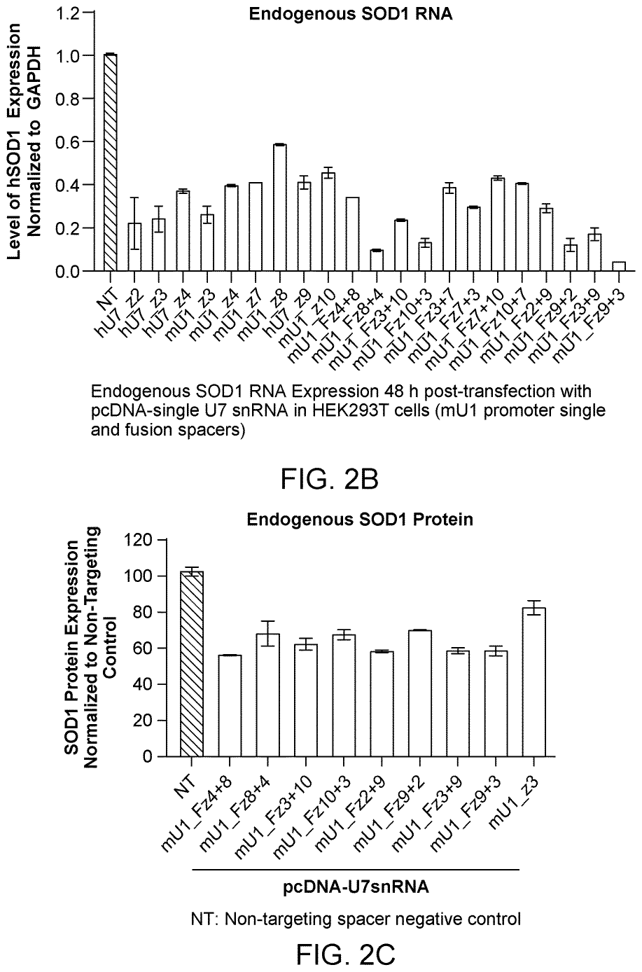

FIG. 2A shows a tapestation image of the RT-PCR products after U7 snRNA treatments using single or fusion U7 spacers expressed under the human U7 (hU7) or mouse U1 (mU1) promoters in HEK293T cells 48 h post-transfection. The gel image shows the SOD1 exon 1 to 4 PCR product (non-skipped band, top), and the skipped products (without exon 2, bottom band. Levels of endogenous human SOD1 RNA expression post-treatment with multiple single and fusion U7 snRNAs (FIG. 2B). SOD1 RNA expression was normalized to GAPDH reference gene and the non-targeting spacer (NT). FIG. 2C shows quantification of the levels of SOD1 endogenous protein in HEK-293T cells treated with U7 snRNAs 48 h post-transfection. SOD1 protein expression was normalized to GAPDH loading control protein levels and normalized to non-targeting control (nt).

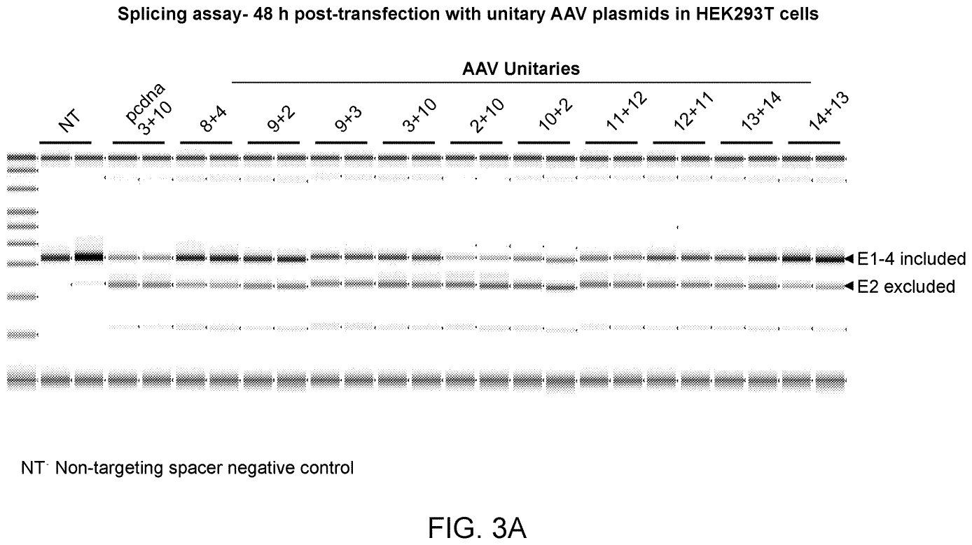

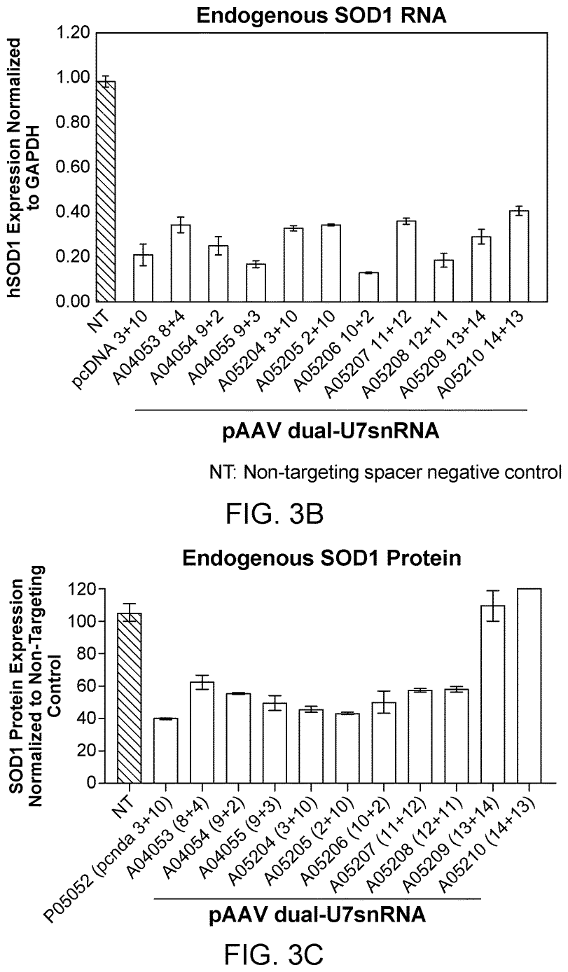

FIG. 3A shows a tapestation image of the RT-PCR products after U7 snRNA treatments using unitary AAV vectors containing dual cassettes expressing fusion U7 spacers expressed under mouse U7 and mouse U1 promoters 48 h post-transfection in HEK293T cells. The gel image shows the SOD1 exon 1 to 4 PCR product (non-skipped band, top), and the skipped product (without exon 2, bottom band). NT (non-targeting U7 was used as a negative control). FIG. 3B depicts levels of endogenous SOD1 RNA expression post-treatment with unitary AAVs expressing dual fusion U7 snRNA cassettes. FIG. 3C shows the levels of SOD1 endogenous protein in HEK-293T cells treated with AAV vectors 72 h post-transfection.

Conclusions: Multiple snRNA spacers promote SOD1 exon 2 skipping and knockdown of endogenous SOD1 mRNA in HEK-293T cells. Fusion spacers show most robust knockdown in vitro of SOD1 mRNA and protein 48 h or 72 h post-transfection.

In Vitro Evaluation of AAVs Encoding U7 snRNAs Targeting SOD1 Exon 2

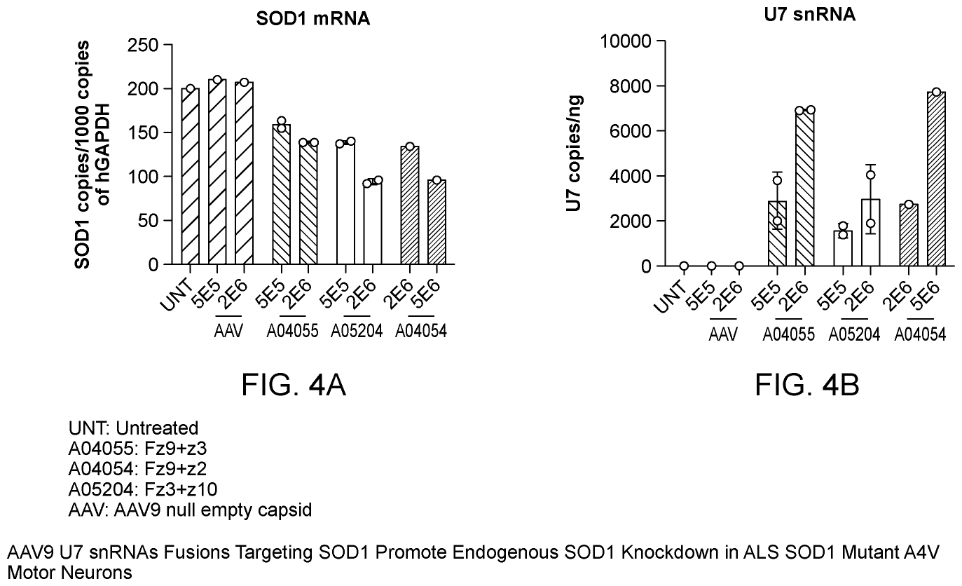

FIG. 4A depicts ddPCR results for SOD1 RNA expression 7 days post-transduction with 5E5, 2E6 or 5E6 vg/cell of AAV9 expressing dual U7 snRNA cassettes (3 different fusion constructs, A04054, Fz9+z2; A04055, Fz9+z3; and A05204, Fz3+z10) in motor neurons with SOD1 ALS mutation A4V. Results are shown as copies of SOD1 RNA per 1000 copies of GAPDH reference gene in untreated (UNT) or snRNA treated cells. FIG. 4B shows quantification of U7 snRNA expression 7 days post-transduction. AAV refers to AAV9 null used as negative control and UNT to untreated cells.

Conclusions: Dose-dependent knockdown of mutant A4V SOD1 RNA 7 days post transduction with AAV9 expressing dual (2×) U7 snRNA cassettes. A05204 (Fz3+z10) and A04054 (Fz9+z2) show most robust knockdown.

In Vivo Evaluation of AAVs Encoding U7 snRNAs Targeting Mus SOD1 Exon 2

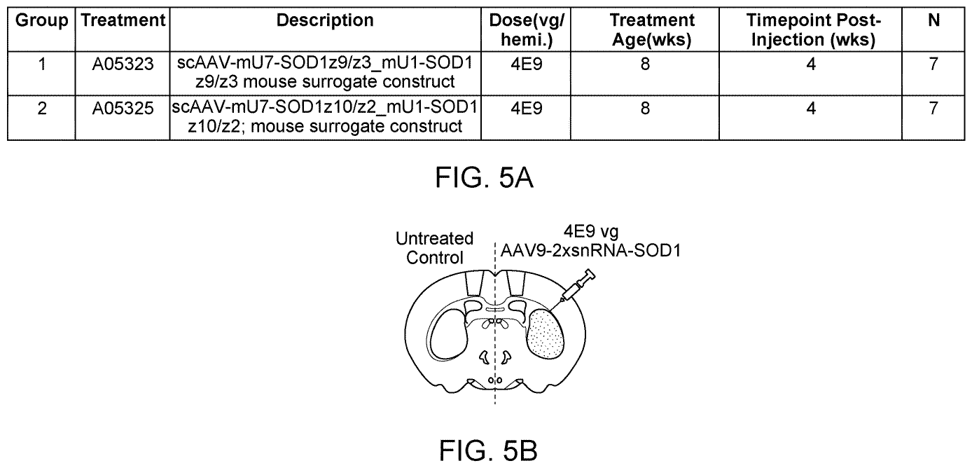

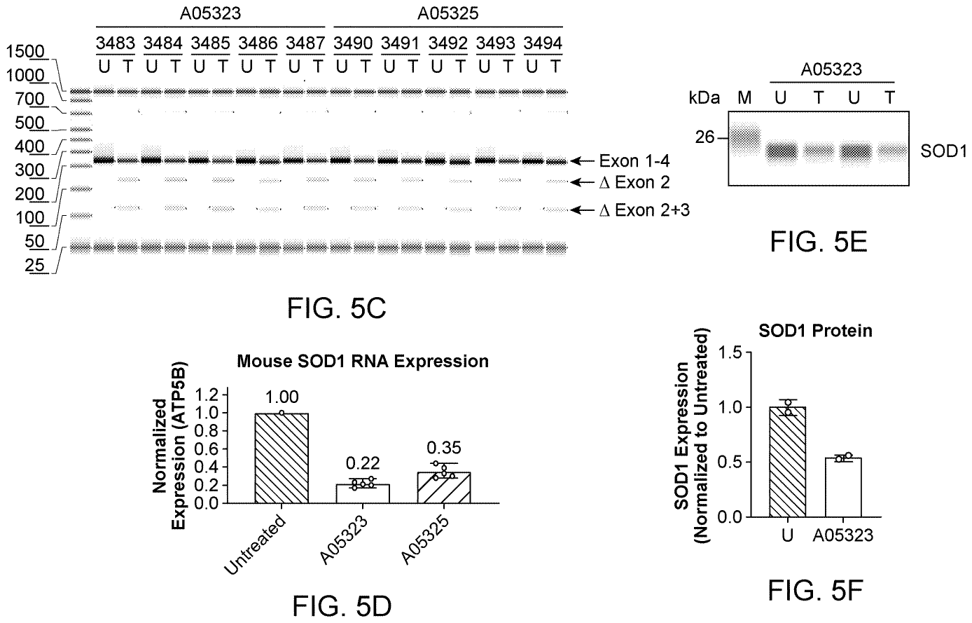

FIGS. 5A-D show study design and results from in vivo studies of C57BL/6 WT mice treated with scAAV9-U7 snRNA targeting mouse SOD1 for Knockdown delivered intrastriatal and harvested 4 weeks post-injection. FIG. 5A shows a table with dose, constructs, timepoint and number of animal used in the study. FIG. 5B diagram showing the schematic injection site of treated vs untreated contralateral striatal sides (with untreated used as a control). Mice were injected unilaterally with 4E9 vg/animal of scAAV9 (containing 2×U7 snRNA cassettes). FIG. 5C is a tapestation image for striatal samples of the RT-PCR products after U7 snRNA treatments using 2 lead constructs A05323 and A05325 (both containing 2×U7 fusion spacer cassettes targeting mouse SOD1), showing the SOD1 excluded products (without exon 2 or without exon 2 and 3; U refers to untreated samples and T snRNA treated). Quantification of the levels of mus (mouse) SOD1 mRNA (FIG. 5D) or protein (FIG. 5E and FIG. 5F) 4-weeks post AAV9 delivery, relative to untreated contralateral controls.

Conclusions: Robust knockdown of SOD1 mRNA (~78% knockdown) and protein (~50% reduction) was observed in vivo 4-weeks post treatment with AAV9-U7 snRNA compared to untreated controls.

In Vitro Evaluation of AAVs Encoding U7 snRNAs Targeting SOD1 Exon 2 for Knockdown and Replacement with Expression of Hardened/Wild-Type SOD1 in A4V SOD1 ALS Mutation Motor neurons

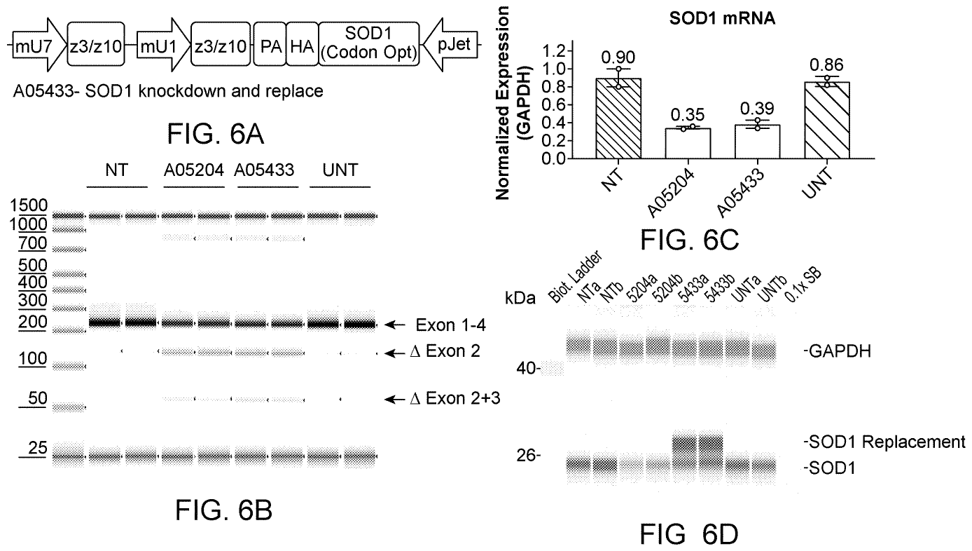

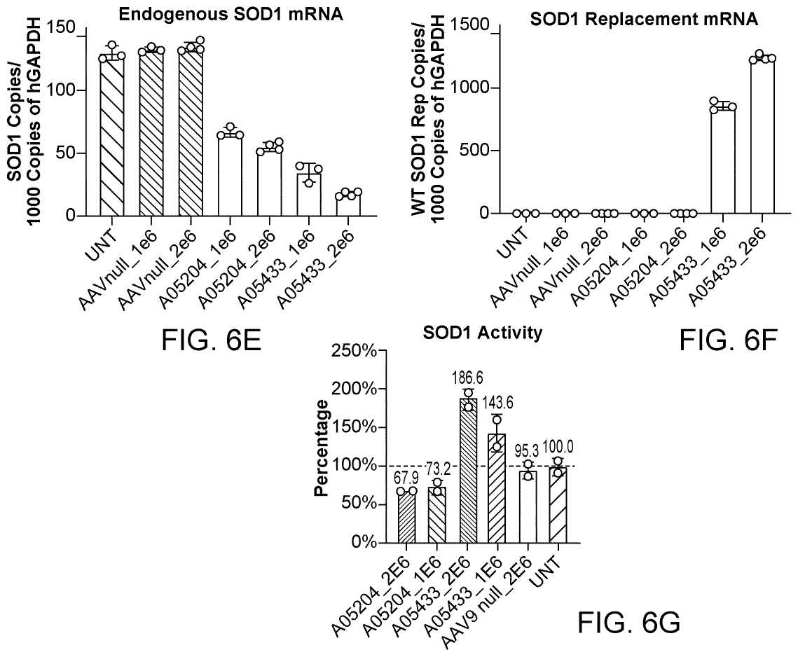

FIGS. 6A-6F show efficacy of SOD1 knockdown and replace construct A05433 transfected in HEK293T cells and transduced as scAAV9 in SOD1 A4V motor neurons. FIG. 6A is a schematic diagram of the AAV plasmid used for SOD1 knockdown and replacement strategy, expressing 2 snRNA cassettes (under mouse U7 and mouse U1 promoters), and the expression of hardened/WT SOD1 (with mutated nucleotides so snRNA is unable to target) under the control of the synthetic pJET promoter being expressed from the antisense strand. FIG. 6B shows a tapestation image of the RT-PCR products after U7 snRNA treatments with the SOD1 exon 1 to 4 PCR product (non-skipped band, top), and the skipped products (skipping of exon 2, middle band; or skipping of exon 2 and 3, bottom band); NT (non-targeting U7 was used as a negative control), dual fusion U7 snRNA (A05204), dual fusion U7 snRNA with SOD1 replacement (A05433), and untreated (UNT). Quantification of human SOD1 mRNA (FIG. 6C) or protein (FIG. 6C) 48 h post-transfection in HEK293T cells. Top band is referent to SOD1_HA tag replacement. FIG. 6E SOD1 RNA expression 7 days post-transduction with 1E6 or 2E6 vg/cell of scAAV9 expressing dual U7 snRNA cassettes (A05204, z3/z10; A05433, z3/z10+SOD1 replacement cassette) in motor neurons with SOD1 ALS mutation A4V. Results are shown as copies of endogenous SOD1 RNA per 1000 copies of GAPDH reference gene. FIG. 6F shows quantification of codon optimized SOD1 replacement transcript and FIG. 6G shows SOD1 enzymatic activity comparison −/+replacement.

Conclusions: Efficient knockdown of endogenous SOD1 RNA and protein with AAV9-U7 snRNA and increase in expression and activity of the hardened/WT replacement SOD1 in a dose-dependent manner in SOD1-ALS motor neurons.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodimented herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

1. An RNA-targeting nucleic acid molecule comprising a small nuclear RNA (snRNA), wherein the snRNA comprises a targeting sequence that binds a SOD1 RNA sequence.

2. The RNA-targeting nucleic acid molecule of claim 1, wherein the SOD1 RNA sequence is an exon 2 sequence.

3. The RNA-targeting nucleic acid molecule of claim 1, wherein the SOD1 targeting sequence comprises a nucleic acid sequence at least 65%, 70%, 75%, 80%, 85%, 90%, 95%, 96%, 97%, 98%, 99% or 100% (or any percentage in between) identical to a nucleic acid sequence set forth in any one of SEQ ID NOs: 1-37.

4. The RNA-targeting nucleic acid molecule of claim 1, wherein the snRNA comprises a stem loop (SL); optionally wherein the SL comprises one or more nucleic acid sequences that are each, independently, at least 65%, 70%, 75%, 80%, 85%, 90%, 95%, 96%, 97%, 98%, 99% or 100% (or any percentage in between) identical to a nucleic acid sequence forth in any one of SEQ ID NOs: 51-91.

5. (canceled)

6. The RNA-targeting nucleic acid molecule of claim 1, wherein the SOD1 RNA sequence is a pre-mRNA or mRNA sequence.

7. The RNA-targeting nucleic acid molecule of claim 1, wherein the snRNA comprises an Sm binding domain (SmBD); optionally wherein the SmBD is a U1, U2, U4, or U5 SmBD.

8-9. (canceled)

10. The RNA-targeting nucleic acid molecule of claim 1, wherein the snRNA comprises a 5′ interaction stabilizer domain (5′ISD); optionally wherein the 5′ISD comprises the nucleotide sequence ggagt, cctct, ggaggt, cctcct, agccag, ggaag, gaagaag, ottg ccgaa, taaggag, gaag, or ggctt.

11. (canceled)

12. An adeno-associated virus (AAV) vector comprising or encoding one or more RNA-targeting nucleic acid molecules of claim 1.

13. (canceled)

14. The AAV vector of claim 12, wherein the snRNA is operably linked to a promoter; optionally wherein the snRNA is operably linked to a U7 promoter or a U1 promoter.

15. (canceled)

16. The AAV vector of claim 12, wherein the snRNA is operably linked to a downstream terminator (DT); optionally wherein the DT is a U7 DT or a U1 DT.

17. (canceled)

18. The AAV vector of claim 12, wherein the vector comprises at least one, at least two, at least three, at least four, or at least five snRNA.

19. (canceled)

20. The AAV vector of claim 18, wherein each snRNA is separated by a buffer sequence; optionally wherein the buffer sequence comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one SEQ ID NOS: 118-124.

21. (canceled)

22. The AAV vector of claim 12, wherein the vector comprises a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 38-50.

23. A SOD1 RNA-targeting nucleic acid molecule comprising a targeting sequence set forth in any one of SEQ ID NOs: 1-37.

24. A polynucleotide or vector comprising a nucleic acid sequence at least 65%, 70%, 75%, 80%, 85%, 90%, 95%, 96%, 97%, 98%, 99% or 100% (or any percentage in between) identical to the nucleic acid sequence set forth in any one of SEQ ID NOs: 38-50.

25. A recombinant AAV (rAAV) comprising:
(a) an AAV capsid comprising an AAV capsid protein; and
(b) a vector genome comprising a sequence encoding the RNA-targeting nucleic acid molecule of claim 1.

26-28. (canceled)

29. The rAAV of claim 25, wherein the AAV capsid comprises an AAV capsid protein of an AAV serotype selected from the group consisting of: AAV1, AAV2, AAV3, AAV4, AAV5, AAV6, AAV7, AAV8, AAV9, AAV10, AAVrh10, AAV11, AAV12, and variants thereof.

30-31. (canceled)

32. A pharmaceutical composition comprising the RNA-targeting nucleic acid molecule of claim 1.

33. A method of targeting one or more target RNAs of interest and exon-skipping the one or more target RNAs, comprising contacting the RNA-targeting nucleic acid molecule of claim 1 with a cell comprising the one or more target RNAs.

34. A method of treating a disease or disorder in a subject in need thereof comprising administering to the subject the RNA-targeting nucleic acid molecule of claim 1.

35-39. (canceled)

40. The RNA-targeting nucleic acid molecule of claim 1, wherein the SOD1 RNA sequence is a wild-type SOD1 RNA sequence or a mutant SOD1 RNA sequence.

41. The RNA-targeting nucleic acid molecule of claim 40, wherein the SOD1 mutation comprises at least one of A4V, H46R, or G93A.

42. A recombinant AAV (rAAV) comprising:
a nucleic acid sequence encoding a first small nuclear RNA (snRNA) molecule, wherein the first snRNA comprises a targeting sequence that binds a target SOD1 RNA sequence; and
a nucleic acid sequence encoding a hardened SOD1 nucleic acid sequence encoding a SOD1 polypeptide.

43. The rAAV of claim 42, further comprising a nucleic acid sequence encoding a second snRNA molecule comprising a targeting sequence that binds a target SOD1 RNA sequence; optionally further comprising a nucleic acid sequence encoding a third snRNA molecule comprising a targeting sequence that binds a target SOD1 RNA sequence; and optionally a fourth snRNA molecule comprising a targeting sequence that binds a target SOD1 RNA sequence.

44. (canceled)

45. The rAAV claim 42, wherein the nucleic acid sequence encoding the hardened SOD1 nucleic acid sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 131.

46-56. (canceled)

57. The rAAV of claim 42, wherein the nucleic acid sequence encoding a hardened SOD1 nucleic acid sequence is expressed from the sense strand of the vector or from the antisense strand of the vector.

58-62. (canceled)


SEQUENCE LISTING (142 sequences)

SEQ ID NO: 1 - RNA, 20 nt, Homo sapiens
  source 1..20: mol_type = other RNA
ctggtccattactttccttt

SEQ ID NO: 2 - RNA, 30 nt, Homo sapiens
  source 1..30: mol_type = other RNA
cacaccttcactggtccattactttccttt

SEQ ID NO: 3 - RNA, 26 nt, Homo sapiens
  source 1..26: mol_type = other RNA
ttccccacaccttcactggtccatta

SEQ ID NO: 4 - RNA, 20 nt, Homo sapiens
  source 1..20: mol_type = other RNA
cccacaccttcactggtcca

SEQ ID NO: 5 - RNA, 26 nt, Homo sapiens
  source 1..26: mol_type = other RNA
ttcagtcagtcctttaatgcttcccc

SEQ ID NO: 6 - RNA, 30 nt, Homo sapiens
  source 1..30: mol_type = other RNA
ccatgcaggccttcagtcagtcctttaatg

SEQ ID NO: 7 - RNA, 30 nt, Homo sapiens
  source 1..30: mol_type = other RNA
gaatccatgcaggccttcagtcagtccttt

SEQ ID NO: 8 - RNA, 20 nt, Homo sapiens
  source 1..20: mol_type = other RNA
ccatgcaggccttcagtcag

SEQ ID NO: 9 - RNA, 26 nt, Homo sapiens
  source 1..26: mol_type = other RNA
gctgtattatctccaaactcatgaac

SEQ ID NO: 10 - RNA, 30 nt, Homo sapiens
  source 1..30: mol_type = other RNA
acctgctgtattatctccaaactcatgaac

SEQ ID NO: 11 - RNA, 26 nt, Homo sapiens
  source 1..26: mol_type = other RNA
ttcatcctttggcccaccgtgttttc

SEQ ID NO: 12 - RNA, 30 nt, Homo sapiens
  source 1..30: mol_type = other RNA
acctctcttcatcctttggcccaccgtgtt

SEQ ID NO: 13 - RNA, 36 nt, Homo sapiens
  source 1..36: mol_type = other RNA
cacaccttcactggtccattactttcctttaagaaa

SEQ ID NO: 14 - RNA, 36 nt, Homo sapiens
  source 1..36: mol_type = other RNA
acacccacctgctgtattatctccaaactcatgaac

SEQ ID NO: 15 - RNA, 36 nt, Homo sapiens
  source 1..36: mol_type = other RNA
tccccacaccttcactggtccattactttcctttaa

SEQ ID NO: 16 - RNA, 36 nt, synthetic construct
  source 1..36: mol_type = other RNA
ccacctgctgtattatctccaaactcatgaacatgg

SEQ ID NO: 17 - RNA, 40 nt, synthetic construct
  source 1..40: mol_type = other RNA
cccacaccttcactggtccaccatgcaggccttcagtcag

SEQ ID NO: 18 - RNA, 40 nt, synthetic construct
  source 1..40: mol_type = other RNA
ccatgcaggccttcagtcagcccacaccttcactggtcca

SEQ ID NO: 19 - RNA, 56 nt, synthetic construct
  source 1..56: mol_type = other RNA
ttccccacaccttcactggtccattaacctgctgtattatctccaaactcatgaac

SEQ ID NO: 20 - RNA, 56 nt, synthetic construct
  source 1..56: mol_type = other RNA
acctgctgtattatctccaaactcatgaacttccccacaccttcactggtccatta

SEQ ID NO: 21 - RNA, 56 nt, synthetic construct
  source 1..56: mol_type = other RNA
ttccccacaccttcactggtccattagaatccatgcaggccttcagtcagtccttt

SEQ ID NO: 22 - RNA, 56 nt, synthetic construct
  source 1..56: mol_type = other RNA
gaatccatgcaggccttcagtcagtcctttttccccacaccttcactggtccatta

SEQ ID NO: 23 - RNA, 60 nt, synthetic construct
  source 1..60: mol_type = other RNA
gaatccatgcaggccttcagtcagtcctttacctgctgtattatctccaaactcatgaac

SEQ ID NO: 24 - RNA, 60 nt, synthetic construct
  source 1..60: mol_type = other RNA
acctgctgtattatctccaaactcatgaacgaatccatgcaggccttcagtcagtccttt

SEQ ID NO: 25 - RNA, 56 nt, synthetic construct
  source 1..56: mol_type = other RNA
cacaccttcactggtccattactttcctttgctgtattatctccaaactcatgaac

SEQ ID NO: 26 - RNA, 56 nt, synthetic construct
  source 1..56: mol_type = other RNA
gctgtattatctccaaactcatgaaccacaccttcactggtccattactttccttt

SEQ ID NO: 27 - RNA, 52 nt, synthetic construct
  source 1..52: mol_type = other RNA
ttccccacaccttcactggtccattagctgtattatctccaaactcatgaac

SEQ ID NO: 28 - RNA, 52 nt, synthetic construct
  source 1..52: mol_type = other RNA
gctgtattatctccaaactcatgaacttccccacaccttcactggtccatta

SEQ ID NO: 29 - RNA, 72 nt, synthetic construct
  source 1..72: mol_type = other RNA
cacaccttcactggtccattactttcctttaagaaaacacccacctgctgtattatctccaaactcatgaac

SEQ ID NO: 30 - RNA, 72 nt, synthetic construct
  source 1..72: mol_type = other RNA
acacccacctgctgtattatctccaaactcatgaaccacaccttcactggtccattactttcctttaagaaa

SEQ ID NO: 31 - RNA, 72 nt, synthetic construct
  source 1..72: mol_type = other RNA
tccccacaccttcactggtccattactttcctttaaccacctgctgtattatctccaaactcatgaacatgg

SEQ ID NO: 32 - RNA, 72 nt, synthetic construct
  source 1..72: mol_type = other RNA
ccacctgctgtattatctccaaactcatgaacatggtccccacaccttcactggtccattactttcctttaa

SEQ ID NO: 33 - RNA, 60 nt, synthetic construct
  source 1..60: mol_type = other RNA
cacaccttcactggtccattactttcctttacctgctgtattatctccaaactcatgaac

SEQ ID NO: 34 - RNA, 60 nt, synthetic construct
  source 1..60: mol_type = other RNA
acctgctgtattatctccaaactcatgaaccacaccttcactggtccattactttccttt

SEQ ID NO: 35 - RNA, 52 nt, synthetic construct
  source 1..52: mol_type = other RNA
tgtgtattgtccccatactgatggacgtcctgacaacacaactggttcaccg

SEQ ID NO: 36 - RNA, 60 nt, synthetic construct
  source 1..60: mol_type = other RNA
accttgtgtattgtccccatactgatggacgacaacacaactggttcaccgcttgccttg

SEQ ID NO: 37 - RNA, 56 nt, synthetic construct; identical to SEQ ID NO: 19
  source 1..56: mol_type = other RNA

SEQ ID NO: 38 - RNA, 1494 nt, synthetic construct
  source 1..1494: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtccatgcaggccttcagtcagcccacaccttcactggtccaaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtccatgcaggccttcagtcagcccacaccttcactggtccaaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 39 - RNA, 1526 nt, synthetic construct
  source 1..1526: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtgctgtattatctccaaactcatgaaccacaccttcactggtccattactttcctttaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtgctgtattatctccaaactcatgaaccacaccttcactggtccattactttcctttaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 40 - RNA, 1518 nt, synthetic construct
  source 1..1518: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtgctgtattatctccaaactcatgaacttccccacaccttcactggtccattaaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtgctgtattatctccaaactcatgaacttccccacaccttcactggtccattaaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 41 - RNA, 1526 nt, synthetic construct
  source 1..1526: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtttccccacaccttcactggtccattaacctgctgtattatctccaaactcatgaacaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtttccccacaccttcactggtccattaacctgctgtattatctccaaactcatgaacaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 42 - RNA, 1534 nt, synthetic construct
  source 1..1534: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtcacaccttcactggtccattactttcctttacctgctgtattatctccaaactcatgaacaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtcacaccttcactggtccattactttcctttacctgctgtattatctccaaactcatgaacaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 43 - RNA, 1534 nt, synthetic construct
  source 1..1534: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtacctgctgtattatctccaaactcatgaaccacaccttcactggtccattactttcctttaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtacctgctgtattatctccaaactcatgaaccacaccttcactggtccattactttcctttaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - RNA, 1558 nt, synthetic construct
  source 1..1558: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtcacaccttcactggtccattactttcctttaagaaaacacccacctgctgtattatctccaaactcatgaacaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtcacaccttcactggtccattactttcctttaagaaaacacccacctgctgtattatctccaaactcatgaacaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 45 - RNA, 1558 nt, synthetic construct
  source 1..1558: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtacacccacctgctgtattatctccaaactcatgaaccacaccttcactggtccattactttcctttaagaaaaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtacacccacctgctgtattatctccaaactcatgaaccacaccttcactggtccattactttcctttaagaaaaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 46 - RNA, 1558 nt, synthetic construct
  source 1..1558: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagttccccacaccttcactggtccattactttcctttaaccacctgctgtattatctccaaactcatgaacatggaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagttccccacaccttcactggtccattactttcctttaaccacctgctgtattatctccaaactcatgaacatggaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 47 - RNA, 1558 nt, synthetic construct
  source 1..1558: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtccacctgctgtattatctccaaactcatgaacatggtccccacaccttcactggtccattactttcctttaaaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtccacctgctgtattatctccaaactcatgaacatggtccccacaccttcactggtccattactttcctttaaaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 48 - RNA, 1518 nt, synthetic construct
  source 1..1518: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagttgtgtattgtccccatactgatggacgtcctgacaacacaactggttcaccgaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagttgtgtattgtccccatactgatggacgtcctgacaacacaactggttcaccgaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 49 - RNA, 1534 nt, synthetic construct
  source 1..1534: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtaccttgtgtattgtccccatactgatggacgacaacacaactggttcaccgcttgccttgaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtaccttgtgtattgtccccatactgatggacgacaacacaactggttcaccgcttgccttgaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 50 - RNA, 2396 nt, synthetic construct
  source 1..2396: mol_type = other RNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagtttccccacaccttcactggtccattaacctgctgtattatctccaaactcatgaacaatttttggagcaggttttctgacctccgtcggaaaacccccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagtttccccacaccttcactggtccattaacctgctgtattatctccaaactcatgaacaatttttggagcaggttttctgacctccgtcggaaaaccgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctaagatacattgatgagtttggacaaaccacaactagaatgcagtgaaaaaaatgctttatttgtgaaatttgtgatgctattgctttatttgtaaccattataagctgcaataaacaagttgttaaggcggccgcgttaagtcaggcgtagtcgggcacatcgtaggggtagtcctcctgggcgattccaatcacgccgcaggccagtctgctgccggcgttgccggtcttggtgctttcctcgttgccgcctttgcccaggtcgtcggccttctcgtgcaccaccagggttctgccgatgatgcagtggtcgccgctcaggctgatcacgctgtcctcgatggacacatcggcgacaccatccttatcagctgtcacatttcccaggtcgcccacgtgccgttcctcgtccttaggtccgccgtgctttctagacagggggttgaagtgagggccggcgcttgtacatccggcggtgttgtcgccgaattcatgaacgtgaaagccgtggaggccctcggtgaggcccttgatagatccccagactttgacggggccgttggattccttctgctcgaagttgatgatgccctgcacagggccatcgcccttcagcacgcacacggctttggtagccatggtggcggcgctagctagttaagcgtgatgtcaagtgacgatcacagggatccacaaacaagaaccgcgacccaaatcccggctgcgacggaactagctgtgccacacccggcgcgtccttatataatcatcggcgttcaccgcccattctccgcccagccataaaaggcaactttcggaacggcgcacgctgattggctccgccctaactccgcccgaattcagttaagtcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 51 - RNA, 27 nt, synthetic construct
  source 1..27: mol_type = other RNA
ggctttctggctccttaccggaaagcc

SEQ ID NO: 52 - RNA, 27 nt, synthetic construct
  source 1..27: mol_type = other RNA
ggctttctgggaggttaccggaaagcc

SEQ ID NO: 53 - RNA, 28 nt, synthetic construct
  source 1..28: mol_type = other RNA
ggctttctggcctccttaccggaaagcc

SEQ ID NO: 54 - RNA, 28 nt, synthetic construct
  source 1..28: mol_type = other RNA
ggctttctggggaggttaccggaaagcc

SEQ ID NO: 55 - RNA, 27 nt, synthetic construct
  source 1..27: mol_type = other RNA
ggctttctggctggctaccggaaagcc

SEQ ID NO: 56 - RNA, 25 nt, synthetic construct
  source 1..25: mol_type = other RNA
ggctttctggcttccccggaaagcc

SEQ ID NO: 57 - RNA, 27 nt, synthetic construct
  source 1..27: mol_type = other RNA
ggctttctggcttcttcccggaaagcc

SEQ ID NO: 58 - RNA, 27 nt, synthetic construct
  source 1..27: mol_type = other RNA
ggctttctggcaacttaccggaaagcc

SEQ ID NO: 59 - RNA, 27 nt, synthetic construct
  source 1..27: mol_type = other RNA
ggctttctggttcggtaccggaaagcc

SEQ ID NO: 60 - RNA, 28 nt, synthetic construct
  source 1..28: mol_type = other RNA
ggctttctggaagccttaccggaaagcc

SEQ ID NO: 61 - RNA, 27 nt, synthetic construct
  source 1..27: mol_type = other RNA
ggctttctggcttcttaccggaaagcc

SEQ ID NO: 62 - RNA, 29 nt, synthetic construct
  source 1..29: mol_type = other RNA
ggctttctggcctccgccggaaagcccct

SEQ ID NO: 63 - RNA, 30 nt, synthetic construct
  source 1..30: mol_type = other RNA
ggctttctggctccttaccggaaagcccct

SEQ ID NO: 64 - RNA, 29 nt, synthetic construct
  source 1..29: mol_type = other RNA
ggttttctgacctccgtcggaaaacccct

SEQ ID NO: 65 - RNA, 33 nt, synthetic construct
  source 1..33: mol_type = other RNA
ggttttctgacctccttcggtcggaaaacccct

SEQ ID NO: 66 - RNA, 26 nt, synthetic construct
  source 1..26: mol_type = other RNA
ggttttctgacctccgtcggaaaacc

SEQ ID NO: 67 - RNA, 30 nt, synthetic construct
  source 1..30: mol_type = other RNA
ggttttctgacactccgtcggaaaacccct

SEQ ID NO: 68 - RNA, 29 nt, synthetic construct
  source 1..29: mol_type = other RNA
ggttttctgatctccatcggaaaacccct

SEQ ID NO: 69 - RNA, 29 nt, synthetic construct
  source 1..29: mol_type = other RNA
ggttttccgacctccgtcggaaaacccct

SEQ ID NO: 70 - RNA, 30 nt, synthetic construct
  source 1..30: mol_type = other RNA
ggctttctggcactccaccggaaagcccct

SEQ ID NO: 71 - RNA, 30 nt, synthetic construct
  source 1..30: mol_type = other RNA
ggctttctggcactccgccggaaagcccct

SEQ ID NO: 72 - RNA, 29 nt, synthetic construct
  source 1..29: mol_type = other RNA
ggctttctggcctccaccggaaagcccct

SEQ ID NO: 73 - RNA, 29 nt, synthetic construct
  source 1..29: mol_type = other RNA
ggttttccggtctccaccggaaagccccc

SEQ ID NO: 74 - RNA, 29 nt, synthetic construct
  source 1..29: mol_type = other RNA
ggctttccggtctccaccggaaagcccct

SEQ ID NO: 75 - RNA, 29 nt, synthetic construct
  source 1..29: mol_type = other RNA
ggctttccggcctccgccggaaagcccct

SEQ ID NO: 76 - RNA, 29 nt, synthetic construct
  source 1..29: mol_type = other RNA
ggttttccggtctccaccggaaaaccctt

SEQ ID NO: 77 - RNA, 29 nt, synthetic construct
  source 1..29: mol_type = other RNA
ggttttccgtgctcccacggaaaaccctt

SEQ ID NO: 78 - RNA, 29 nt, synthetic construct
  source 1..29: mol_type = other RNA
ggttttccggcctccgccggaaaaccctt

SEQ ID NO: 79 - RNA, 30 nt, synthetic construct
  source 1..30: mol_type = other RNA
ggttttccgtgactcccacggaaaaccctt

SEQ ID NO: 80 - RNA, 30 nt, synthetic construct
  source 1..30: mol_type = other RNA
ggttttccggcactccgccggaaaaccctt

SEQ ID NO: 81 - RNA, 29 nt, synthetic construct
  source 1..29: mol_type = other RNA
ggtcttccggtctcctccggaaggccccc

SEQ ID NO: 82 - RNA, 27 nt, synthetic construct
  source 1..27: mol_type = other RNA
ggtcttccggctccccggaaggccccc

SEQ ID NO: 83 - RNA, 29 nt, synthetic construct
  source 1..29: mol_type = other RNA
ggctttccgtgctcccacggaaagcccct

SEQ ID NO: 84 - RNA, 30 nt, synthetic construct
  source 1..30: mol_type = other RNA
ggctttccgtgactcccacggaaagcccct

SEQ ID NO: 85 - RNA, 30 nt, synthetic construct
  source 1..30: mol_type = other RNA
ggctttccggcactccgccggaaagcccct

SEQ ID NO: 86 - RNA, 29 nt, synthetic construct
  source 1..29: mol_type = other RNA
ggttttctgacttcggtcggaaaacccct

SEQ ID NO: 87 - RNA, 26 nt, synthetic construct
  source 1..26: mol_type = other RNA
ggttttctgacttcggtcggaaaacc

SEQ ID NO: 88 - RNA, 27 nt, synthetic construct
  source 1..27: mol_type = other RNA
ggctttctggctttttaccggaaagcc

SEQ ID NO: 89 - RNA, 30 nt, synthetic construct
  source 1..30: mol_type = other RNA
ggctttctggctttttaccggaaagcccct

SEQ ID NO: 90 - RNA, 29 nt, synthetic construct
  source 1..29: mol_type = other RNA
ggctttccggcctccgccggaaagcccct

SEQ ID NO: 91 - RNA, 26 nt, synthetic construct
  source 1..26: mol_type = other RNA
ggctttccggcctccgccggaaagcc

SEQ ID NO: 92 - DNA, 392 bp, Homo sapiens
  source 1..392: mol_type = genomic DNA
aaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttc

SEQ ID NO: 93 - DNA, 247 bp, Homo sapiens
  source 1..247: mol_type = genomic DNA
ccacgccctctgtgaaagggcggggcatgcaaattcgaaatgaaagcccgggaacgccggaagaagcacgggtgtaagatttcccttttcaaaggcagagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgc

SEQ ID NO: 94 - DNA, 250 bp, Homo sapiens
  source 1..250: mol_type = genomic DNA
ttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaatt

SEQ ID NO: 95 - DNA, 300 bp, Homo sapiens
  source 1..300: mol_type = genomic DNA
gcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggt

SEQ ID NO: 96 - DNA, 296 bp, Homo sapiens
  source 1..296: mol_type = genomic DNA
gtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 97 - DNA, 243 bp, Homo sapiens
  source 1..243: mol_type = genomic DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 98 - DNA, 106 bp, Homo sapiens
  source 1..106: mol_type = genomic DNA
caggactagtcttttaggtcaaaaagaagaagctttgtaaccgttggtttccgtagtgtagtggttatcacgttcgcctaacacgcgaaaggtccccggttcgaag

SEQ ID NO: 99 - DNA, 439 bp, Mus musculus
  source 1..439: mol_type = genomic DNA
ttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 100 - DNA, 258 bp, Mus musculus
  source 1..258: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaa

SEQ ID NO: 101 - DNA, 256 bp, Mus musculus
  source 1..256: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 102 - DNA, 288 bp, Homo sapiens
  source 1..288: mol_type = genomic DNA
tactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 103 - DNA, 440 bp, Mus musculus
  source 1..440: mol_type = genomic DNA
tgccaacagctttgaaatcctctgtgctcttgtgccaatcccaccctcagaacagggagtctgtcggttatgttttcgtcctttacagttcttcgccgtctaatccattggaaacctcctttatttaaaggaaaccgtgtccaaattcagtgtacagaggtgcaaacctccgccccgctccttgtaggaagcccacgggaactgggccaatcggacggcgcttactcggctcatttacatacccataacacaccgcggctaatgcaaatattttcgtgaaaagatcttccattactcgaagttttaaaactaaaaacaaaatattatttcaaaagatcatgaagaaatttctgtcggtaacagcagtttcaattgataaatcaccatcacttatctaggaggttctgttactctagaagtgaattaagcaggacagctgt

SEQ ID NO: 104 - DNA, 231 bp, Mus musculus
  source 1..231: mol_type = genomic DNA
gggcggggcatgcaaataactgctctgtggaactctgggagcaaaaacaaaaaactgcaaccaaaacttctcggcctccttgaatcttacaggcttttcgtggcgtaaaggtggtgtactcaatgaagaggagagtctgtgttggctgcatgtttgagtcggttggttggtgactgtgaattaaaggtgtggtcggtgttgagtgtatggggcgtgtgggcgtagttcggt

SEQ ID NO: 105 - DNA, 311 bp, Mus musculus
  source 1..311: mol_type = genomic DNA
cgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttt

SEQ ID NO: 106 - DNA, 97 bp, Mus musculus
  source 1..97: mol_type = genomic DNA
catgcaaattacgcgctgtgctttgtgggaaatcaccctaaacgtaaaatttattcctctttcgagccttatagtggcggccggtctacaccctaaa

SEQ ID NO: 107 - DNA, 50 bp, Homo sapiens
  source 1..50: mol_type = genomic DNA
actttctggagtttcaaaaacagactgtacgccaagggtcatatcttttt

SEQ ID NO: 108 - DNA, 50 bp, Homo sapiens
  source 1..50: mol_type = genomic DNA
ccggggatacaacgtgtttcctaaaagtagagggaggtaagagacggtag

SEQ ID NO: 109 - DNA, 50 bp, Homo sapiens
  source 1..50: mol_type = genomic DNA
ctgaattttcttgcagttgaacaacagaggctttttttgtgtgtgtgggg

SEQ ID NO: 110 - DNA, 50 bp, Homo sapiens
  source 1..50: mol_type = genomic DNA
atatgtggtaatccaacaatagaaattatttttaagtttgtgtgttcctt

SEQ ID NO: 111 - DNA, 206 bp, Mus musculus
  source 1..206: mol_type = genomic DNA
gtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagca

SEQ ID NO: 112 - DNA, 110 bp, Mus musculus
  source 1..110: mol_type = genomic DNA
cccaatttcactggtctacaatgaaagcaaaacagttctcttccccgctccccggtgtgtgagaggggctttgatccttctctggtttcctaggaaacgcgtatgtgtac

SEQ ID NO: 113 - DNA, 107 bp, Mus musculus
  source 1..107: mol_type = genomic DNA
cccaatttcactggtctacaatgaaagcaaaacagttctcttccccgctccccggtgtgtgagaggggctttgatccttctctggtttcctaggaaacgcgtatgtg

SEQ ID NO: 114 - DNA, 94 bp, Homo sapiens
  source 1..94: mol_type = genomic DNA
cctcttatgatgtttgttgccaatgatagattgttttcactgtgcaaaaattatgggtagttttggtggtcttgatgcagttgtaagcttggag

SEQ ID NO: 115 - DNA, 91 bp, Homo sapiens
  source 1..91: mol_type = genomic DNA
cttatgatgtttgttgccaatgatagattgttttcactgtgcaaaaattatgggtagttttggtggtcttgatgcagttgtaagcttggag

SEQ ID NO: 116 - DNA, 200 bp, Mus musculus
  source 1..200: mol_type = genomic DNA
ccacattttgtgtttaaaaatagaaatatttaagtgagatcagtttaaatctgctttatctagggtgtctaactgcttgcatctttttaacttttccttatttgtgagaaggtctgtaaaacttattatatgttagtacactgtagctgtcttcagacactccagaagagggattcagatcaccttacagatggttttga

SEQ ID NO: 117 - DNA, 78 bp, Mus musculus
  source 1..78: mol_type = genomic DNA
ccctctggggagtaaagttggttttaaagtcagagcatggtgattgtagggcagtccaacttttttaaatatgctgtg

SEQ ID NO: 118 - DNA, 30 bp, synthetic construct
  source 1..30: mol_type = other DNA
caaactacagagccaagtgctatccacaga

SEQ ID NO: 119 - DNA, 30 bp, synthetic construct
  source 1..30: mol_type = other DNA
gagctttctgggttgccatctcaagcagac

SEQ ID NO: 120 - DNA, 30 bp, synthetic construct
  source 1..30: mol_type = other DNA
tacaaggccatcagctcatactcacaattg

SEQ ID NO: 121 - DNA, 100 bp, synthetic construct
  source 1..100: mol_type = other DNA
caaactacagagccaagtgctatccacagagagctttctgggttgccatctcaagcagactacaaggccatcagctcatactcacaattgactttgagag

SEQ ID NO: 122 - DNA, 100 bp, synthetic construct
  source 1..100: mol_type = other DNA
ttgaccacatacgtgctctttcaaagttctgtgtttgaagttatgttagtaacaactgatgcccatcctgcaatgacaaatccaattctcagtgcagctc

SEQ ID NO: 123 - DNA, 500 bp, synthetic construct
  source 1..500: mol_type = other DNA
caaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgt

SEQ ID NO: 124 - DNA, 500 bp, synthetic construct
  source 1..500: mol_type = other DNA
ttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaac

SEQ ID NO: 125 - DNA, 110 bp, synthetic construct
  source 1..110: mol_type = other DNA
ctgcgcgctcgctcgctcactgaggccgcccgggcaaagcccgggcgtcgggcgacctttggtcgcccggcctcagtgagcgagcgagcgcgcagagagggagtggggtt

SEQ ID NO: 126 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 127 - RNA, 13 nt, synthetic construct
  source 1..13: mol_type = other RNA
aatttttggagca

SEQ ID NO: 128 - RNA, 13 nt, synthetic construct
  source 1..13: mol_type = other RNA
aatttttggagta

SEQ ID NO: 129 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 130 - DNA, 27 bp, synthetic construct
  source 1..27: mol_type = other DNA
ggcgtagtcgggcacatcgtaggggta

SEQ ID NO: 131 - DNA, 462 bp, synthetic construct
  source 1..462: mol_type = other DNA
ctgggcgattccaatcacgccgcaggccagtctgctgccggcgttgccggtcttggtgctttcctcgttgccgcctttgcccaggtcgtcggccttctcgtgcaccaccagggttctgccgatgatgcagtggtcgccgctcaggctgatcacgctgtcctcgatggacacatcggcgacaccatccttatcagctgtcacatttcccaggtcgcccacgtgccgttcctcgtccttaggtccgccgtgctttctagacagggggttgaagtgagggccggcgcttgtacatccggcggtgttgtcgccgaattcatgaacgtgaaagccgtggaggccctcggtgaggcccttgatagatccccagactttgacggggccgttggattccttctgctcgaagttgatgatgccctgcacagggccatcgcccttcagcacgcacacggctttggtagccat

SEQ ID NO: 132 - DNA, 195 bp, synthetic construct
  source 1..195: mol_type = other DNA
tgtcaagtgacgatcacagggatccacaaacaagaaccgcgacccaaatcccggctgcgacggaactagctgtgccacacccggcgcgtccttatataatcatcggcgttcaccgcccattctccgcccagccataaaaggcaactttcggaacggcgcacgctgattggctccgccctaactccgcccgaattc

SEQ ID NO: 133 - RNA, 895 nt, Homo sapiens
  source 1..895: mol_type = genomic RNA
gcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaa

SEQ ID NO: 134 - protein, 153 aa, Homo sapiens
ATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

SEQ ID NO: 135 - DNA, 420 bp, Rattus norvegicus
  source 1..420: mol_type = genomic DNA
ttacttcatactaaaggctgtgcatccgactcctaagttgatgaaggaaaatgcctagtgttcttggaggctacaaaacaaaagacaaagctaactaccatctgcttatgggttcattggtattttccagctggcagggaggcgggtttccgagtacaggaaatgagtctctatggaggcggtgctatgtagatgagaattcaggagcaaactgggaaaagcagctgcttccaaatatttgtgatttttggagtgtagttttggggaaactcgcagcctaccaattctcctaagtgctttagaatatggagagacactgtacataaagatatagagctttttaatggaggcttaaatttataccgtatctacaaaatgctacattcacaatgcagttcaggctctgtggcattgcaactc

SEQ ID NO: 136 - DNA, 306 bp, Bos taurus
  source 1..306: mol_type = genomic DNA
gcgcaggagccgccgagctcttgctgcgaagccttgtctgcgttcttaaaaactaaagggggcgtgactggcttccttatcagccaatcggcatcgggtcatttgcatagggccccatccaccgttcgtaaactctagccgcgaaggatttaagagagttccttagaacacggtcttcccctccgaaagtcgcagtctgcctcgttatctgtagagcaataggagctaggaaccggccgtcggggctcaccctcactgacagcggggttggtggctatgccgtgcgatctccgggtgctacgtctg

SEQ ID NO: 137 - DNA, 420 bp, synthetic construct
  source 1..420: mol_type = other DNA
cgccgtgccctgctctgccctccgcacgctgctcagactccacacccgtaacgaagctccaccgaatttttcgtctgtctgtgaagacgaagaattgaaaaatctcagctacacttggtctttggggtcggcgccaggcagagctagaacgaactagacgcttgtccgaaagcagtgacgtcaaatgacaagggacaagaggtggggatatgtagatgaggggagcggtggctgctggctggatttcgtctttcgccgggtagaatttcccctgaacgtccaaaggatgtatcttggtcactgtgaaataaataatccttcaggttcccatcgaaggccgaaatagcttggaatataattgaccataactctgttgtgtgctgagttcgagggtgacgtcccagactaagcttacaactc

SEQ ID NO: 138 - DNA, 420 bp, Equus caballus
  source 1..420: mol_type = genomic DNA
tgccgcgcagaatgcagactcatcctgacaagtccacgccagaacctttgcgtctgcccggaaaggcaaggaagtttaaagcctttccagatacccctatggatgattgtcttttagatcgtcctgagggctagctagagcgaattcaccgcttctccgagaagcagaatgacgtcgagtgacgacagtgatgacaagaggcggggatatgtagatgaaagacacggtgactgccggttggagtgagttcttcatggggaacactttccactgaatatccaaaaggatgtaccctgatctctgtcatataaataacccttcaatttcgaacttaaggcctaaagaactcggaatataattgaccatgactttatagtgtgttgaagacgatggtgaggtgccaggcgaggggagcaactc

SEQ ID NO: 139 - DNA, 200 bp, Rattus norvegicus
  source 1..200: mol_type = genomic DNA
atttgtttggtactaaagatagttatcagccgaaccagaaggctaaaatggcttctgatacttacttggccaatgccttttccctttatactgctattgctttgtattctgaaaagcatcttagtggtttttaactttctctacgttccctctgcacgttgagtcttgagttatgttaaatggtacggtacctaggattg

SEQ ID NO: 140 - DNA, 200 bp, Bos taurus
  source 1..200: mol_type = genomic DNA
cccacacaagtgtttatgaacaatagaataatatgtgggtgggaagcttggttttttttgatacgggttttttttttttttttggtctacgtgtttagtccgttcttgacggagaagacccgggaggttgggattcttgtctggaaatttccggctctgtctgatgtagtttcccgttcgcctcccttctttgctgcgag

SEQ ID NO: 141 - DNA, 200 bp, synthetic construct
  source 1..200: mol_type = other DNA
gcgtggtttgtgcaaggaaagacatagatgctaagtttggagctctgctaatttcctgtttttgtagtcgttttcgggtttatgaggcagaggcagtaagcagttttcctgcaacatggataagctagtttggacttttggtaaaggatttttttgtaatgtgggagacttgcttttgatccctggtttgggaaattctc

SEQ ID NO: 142 - DNA, 200 bp, Equus caballus
  source 1..200: mol_type = genomic DNA
acaaacttggtcttaaaagaagactacttgtttatgttcttctttctctaacttttatttggggtttagtggctttctaacatggtcattatgaggtaagaccttggggttggcctattaattaacaccttggggctggccgtatggctgagtggttaagtttgtgtgctctgcttcgggtttggcctgttctaattctg